SEQUENCE LISTING (227 sequences)

SEQ ID NO: 1 - protein, 15 aa, Artificial sequence
  Synthetic sequence Peptide derivative
LQFTTIASLFAAAGL

SEQ ID NO: 2 - protein, 15 aa, Alternaria alternata
FTTIASLFAAAGLAA

SEQ ID NO: 3 - protein, 15 aa, Alternaria alternata
IASLFAAAGLAAAAP

SEQ ID NO: 4 - protein, 15 aa, Alternaria alternata
ASLFAAAGLAAAAPL

SEQ ID NO: 5 - protein, 15 aa, Alternaria alternata
AAGLAAAAPLESRQD

SEQ ID NO: 6 - protein, 15 aa, Alternaria alternata
EGDYVWKISEFYGRK

SEQ ID NO: 7 - protein, 15 aa, Alternaria alternata
DYVWKISEFYGRKPE

SEQ ID NO: 8 - protein, 15 aa, Alternaria alternata
ISEFYGRKPEGTYYN

SEQ ID NO: 9 - protein, 15 aa, Alternaria alternata
SEFYGRKPEGTYYNS

SEQ ID NO: 10 - protein, 15 aa, Alternaria alternata
EGTYYNSLGFNIKAT

SEQ ID NO: 11 - protein, 15 aa, Alternaria alternata
GTYYNSLGFNIKATN

SEQ ID NO: 12 - protein, 15 aa, Alternaria alternata
YNSLGFNIKATNGGT

SEQ ID NO: 13 - protein, 15 aa, Alternaria alternata
SLGFNIKATNGGTLD

SEQ ID NO: 14 - protein, 15 aa, Alternaria alternata
GFNIKATNGGTLDFT

SEQ ID NO: 15 - protein, 15 aa, Alternaria alternata
GGTLDFTVSAQADKL

SEQ ID NO: 16 - protein, 15 aa, Artificial sequence
  Synthetic sequence Peptide derivative
DHKWYSVGENSFLDF

SEQ ID NO: 17 - protein, 15 aa, Alternaria alternata
RSGLLLKQKVSDDIT

SEQ ID NO: 18 - protein, 15 aa, Alternaria alternata
SGLLLKQKVSDDITY

SEQ ID NO: 19 - protein, 15 aa, Alternaria alternata
KQKVSDDITYVATAT

SEQ ID NO: 20 - protein, 15 aa, Alternaria alternata
SDDITYVATATLPNY

SEQ ID NO: 21 - protein, 15 aa, Artificial sequence
  Synthetic sequence Peptide derivative
DITYVATATLPNYVR

SEQ ID NO: 22 - protein, 15 aa, Artificial sequence
  Synthetic sequence Peptide derivative
TATLPNYVRAGGNGP

SEQ ID NO: 23 - protein, 15 aa, Artificial sequence
  Synthetic sequence Peptide derivative
LPNYVRAGGNGPKDF

SEQ ID NO: 24 - protein, 15 aa, Artificial sequence
  Synthetic sequence Peptide derivative
PKDFVVQGVADAYIT

SEQ ID NO: 25 - protein, 15 aa, Alternaria alternata
VADAYITLVTLPKSS

SEQ ID NO: 26 - protein, 15 aa, Alternaria alternata
AAYLLLGLGGNTSPS

SEQ ID NO: 27 - protein, 12 aa, Alternaria alternata
FTTIASLFAAAG

SEQ ID NO: 28 - protein, 11 aa, Alternaria alternata
TTIASLFAAAG

SEQ ID NO: 29 - protein, 10 aa, Alternaria alternata
TIASLFAAAG

SEQ ID NO: 30 - protein, 12 aa, Alternaria alternata
IASLFAAAGLAA

SEQ ID NO: 31 - protein, 11 aa, Alternaria alternata
IASLFAAAGLA

SEQ ID NO: 32 - protein, 10 aa, Alternaria alternata
IASLFAAAGL

SEQ ID NO: 33 - protein, 14 aa, Alternaria alternata
FTTIASLFAAAGLA

SEQ ID NO: 34 - protein, 13 aa, Alternaria alternata
FTTIASLFAAAGL

SEQ ID NO: 35 - protein, 14 aa, Alternaria alternata
TTIASLFAAAGLAA

SEQ ID NO: 36 - protein, 13 aa, Alternaria alternata
TTIASLFAAAGLA

SEQ ID NO: 37 - protein, 12 aa, Alternaria alternata
TTIASLFAAAGL

SEQ ID NO: 38 - protein, 13 aa, Alternaria alternata
TIASLFAAAGLAA

SEQ ID NO: 39 - protein, 12 aa, Alternaria alternata
TIASLFAAAGLA

SEQ ID NO: 40 - protein, 11 aa, Alternaria alternata
TIASLFAAAGL

SEQ ID NO: 41 - protein, 9 aa, Alternaria alternata
IASLFAAAG

SEQ ID NO: 42 - protein, 12 aa, Alternaria alternata
ASLFAAAGLAAA

SEQ ID NO: 43 - protein, 11 aa, Alternaria alternata
SLFAAAGLAAA

SEQ ID NO: 44 - protein, 10 aa, Alternaria alternata
LFAAAGLAAA

SEQ ID NO: 45 - protein, 12 aa, Alternaria alternata
FAAAGLAAAAPL

SEQ ID NO: 46 - protein, 11 aa, Alternaria alternata
FAAAGLAAAAP

SEQ ID NO: 47 - protein, 10 aa, Alternaria alternata
FAAAGLAAAA

SEQ ID NO: 48 - protein, 14 aa, Alternaria alternata
ASLFAAAGLAAAAP

SEQ ID NO: 49 - protein, 13 aa, Alternaria alternata
ASLFAAAGLAAAA

SEQ ID NO: 50 - protein, 14 aa, Alternaria alternata
SLFAAAGLAAAAPL

SEQ ID NO: 51 - protein, 13 aa, Alternaria alternata
SLFAAAGLAAAAP

SEQ ID NO: 52 - protein, 12 aa, Alternaria alternata
SLFAAAGLAAAA

SEQ ID NO: 53 - protein, 13 aa, Alternaria alternata
LFAAAGLAAAAPL

SEQ ID NO: 54 - protein, 12 aa, Alternaria alternata
LFAAAGLAAAAP

SEQ ID NO: 55 - protein, 11 aa, Alternaria alternata
LFAAAGLAAAA

SEQ ID NO: 56 - protein, 9 aa, Alternaria alternata
FAAAGLAAA

SEQ ID NO: 57 - protein, 12 aa, Alternaria alternata
AAGLAAAAPLES

SEQ ID NO: 58 - protein, 11 aa, Alternaria alternata
AGLAAAAPLES

SEQ ID NO: 59 - protein, 10 aa, Alternaria alternata
GLAAAAPLES

SEQ ID NO: 60 - protein, 12 aa, Alternaria alternata
LAAAAPLESRQD

SEQ ID NO: 61 - protein, 11 aa, Alternaria alternata
LAAAAPLESRQ

SEQ ID NO: 62 - protein, 10 aa, Alternaria alternata
LAAAAPLESR

SEQ ID NO: 63 - protein, 14 aa, Alternaria alternata
AAGLAAAAPLESRQ

SEQ ID NO: 64 - protein, 13 aa, Alternaria alternata
AAGLAAAAPLESR

SEQ ID NO: 65 - protein, 14 aa, Alternaria alternata
AGLAAAAPLESRQD

SEQ ID NO: 66 - protein, 13 aa, Alternaria alternata
AGLAAAAPLESRQ

SEQ ID NO: 67 - protein, 12 aa, Alternaria alternata
AGLAAAAPLESR

SEQ ID NO: 68 - protein, 13 aa, Alternaria alternata
GLAAAAPLESRQD

SEQ ID NO: 69 - protein, 12 aa, Alternaria alternata
GLAAAAPLESRQ

SEQ ID NO: 70 - protein, 11 aa, Alternaria alternata
GLAAAAPLESR

SEQ ID NO: 71 - protein, 9 aa, Alternaria alternata
LAAAAPLES

SEQ ID NO: 72 - protein, 12 aa, Alternaria alternata
GTYYNSLGFNIK

SEQ ID NO: 73 - protein, 11 aa, Alternaria alternata
TYYNSLGFNIK

SEQ ID NO: 74 - protein, 10 aa, Alternaria alternata
YYNSLGFNIK

SEQ ID NO: 75 - protein, 12 aa, Alternaria alternata
YNSLGFNIKATN

SEQ ID NO: 76 - protein, 11 aa, Alternaria alternata
YNSLGFNIKAT

SEQ ID NO: 77 - protein, 10 aa, Alternaria alternata
YNSLGFNIKA

SEQ ID NO: 78 - protein, 14 aa, Alternaria alternata
GTYYNSLGFNIKAT

SEQ ID NO: 79 - protein, 13 aa, Alternaria alternata
GTYYNSLGFNIKA

SEQ ID NO: 80 - protein, 14 aa, Alternaria alternata
TYYNSLGFNIKATN

SEQ ID NO: 81 - protein, 13 aa, Alternaria alternata
TYYNSLGFNIKAT

SEQ ID NO: 82 - protein, 12 aa, Alternaria alternata
TYYNSLGFNIKA

SEQ ID NO: 83 - protein, 13 aa, Alternaria alternata
YYNSLGFNIKATN

SEQ ID NO: 84 - protein, 12 aa, Alternaria alternata
YYNSLGFNIKAT

SEQ ID NO: 85 - protein, 11 aa, Alternaria alternata
YYNSLGFNIKA

SEQ ID NO: 86 - protein, 9 aa, Alternaria alternata
YNSLGFNIK

SEQ ID NO: 87 - protein, 12 aa, Alternaria alternata
YNSLGFNIKATN

SEQ ID NO: 88 - protein, 11 aa, Alternaria alternata
NSLGFNIKATN

SEQ ID NO: 89 - protein, 10 aa, Alternaria alternata
SLGFNIKATN

SEQ ID NO: 90 - protein, 12 aa, Alternaria alternata
LGFNIKATNGGT

SEQ ID NO: 91 - protein, 11 aa, Alternaria alternata
LGFNIKATNGG

SEQ ID NO: 92 - protein, 10 aa, Alternaria alternata
LGFNIKATNG

SEQ ID NO: 93 - protein, 14 aa, Alternaria alternata
YNSLGFNIKATNGG

SEQ ID NO: 94 - protein, 13 aa, Alternaria alternata
YNSLGFNIKATNG

SEQ ID NO: 95 - protein, 14 aa, Alternaria alternata
NSLGFNIKATNGGT

SEQ ID NO: 96 - protein, 13 aa, Alternaria alternata
NSLGFNIKATNGG

SEQ ID NO: 97 - protein, 12 aa, Alternaria alternata
NSLGFNIKATNG

SEQ ID NO: 98 - protein, 13 aa, Alternaria alternata
SLGFNIKATNGGT

SEQ ID NO: 99 - protein, 12 aa, Alternaria alternata
SLGFNIKATNGG

SEQ ID NO: 100 - protein, 11 aa, Alternaria alternata
SLGFNIKATNG

SEQ ID NO: 101 - protein, 9 aa, Alternaria alternata
LGFNIKATN

SEQ ID NO: 102 - protein, 12 aa, Alternaria alternata
SDDITYVATATL

SEQ ID NO: 103 - protein, 11 aa, Alternaria alternata
DDITYVATATL

SEQ ID NO: 104 - protein, 10 aa, Alternaria alternata
DITYVATATL

SEQ ID NO: 105 - protein, 12 aa, Alternaria alternata
ITYVATATLPNY

SEQ ID NO: 106 - protein, 11 aa, Alternaria alternata
ITYVATATLPN

SEQ ID NO: 107 - protein, 10 aa, Alternaria alternata
ITYVATATLP

SEQ ID NO: 108 - protein, 14 aa, Alternaria alternata
SDDITYVATATLPN

SEQ ID NO: 109 - protein, 13 aa, Alternaria alternata
SDDITYVATATLP

SEQ ID NO: 110 - protein, 14 aa, Alternaria alternata
DDITYVATATLPNY

SEQ ID NO: 111 - protein, 13 aa, Alternaria alternata
DDITYVATATLPN

SEQ ID NO: 112 - protein, 12 aa, Alternaria alternata
DDITYVATATLP

SEQ ID NO: 113 - protein, 13 aa, Alternaria alternata
DITYVATATLPNY

SEQ ID NO: 114 - protein, 12 aa, Alternaria alternata
DITYVATATLPN

SEQ ID NO: 115 - protein, 11 aa, Alternaria alternata
DITYVATATLP

SEQ ID NO: 116 - protein, 9 aa, Alternaria alternata
ITYVATATL

SEQ ID NO: 117 - protein, 12 aa, Alternaria alternata
DITYVATATLPN

SEQ ID NO: 118 - protein, 11 aa, Alternaria alternata
ITYVATATLPN

SEQ ID NO: 119 - protein, 10 aa, Alternaria alternata
TYVATATLPN

SEQ ID NO: 120 - protein, 12 aa, Artificial sequence
  Synthetic sequence Peptide derivative
YVATATLPNYVR

SEQ ID NO: 121 - protein, 11 aa, Artificial sequence
  Synthetic sequence Peptide derivative
YVATATLPNYV

SEQ ID NO: 122 - protein, 10 aa, Alternaria alternata
YVATATLPNY

SEQ ID NO: 123 - protein, 14 aa, Artificial sequence
  Synthetic sequence Peptide derivative
DITYVATATLPNYV

SEQ ID NO: 124 - protein, 13 aa, Alternaria alternata
DITYVATATLPNY

SEQ ID NO: 125 - protein, 14 aa, Artificial sequence
  Synthetic sequence Peptide derivative
ITYVATATLPNYVR

SEQ ID NO: 126 - protein, 13 aa, Artificial sequence
  Synthetic sequence Peptide derivative
ITYVATATLPNYV

SEQ ID NO: 127 - protein, 12 aa, Alternaria alternata
ITYVATATLPNY

SEQ ID NO: 128 - protein, 13 aa, Artificial sequence
  Synthetic sequence Peptide derivative
TYVATATLPNYVR

SEQ ID NO: 129 - protein, 12 aa, Artificial sequence
  Synthetic sequence Peptide derivative
TYVATATLPNYV

SEQ ID NO: 130 - protein, 11 aa, Alternaria alternata
TYVATATLPNY

SEQ ID NO: 131 - protein, 9 aa, Alternaria alternata
YVATATLPN

SEQ ID NO: 132 - protein, 12 aa, Alternaria alternata
YVATATLPNYCR

SEQ ID NO: 133 - protein, 11 aa, Alternaria alternata
YVATATLPNYC

SEQ ID NO: 134 - protein, 15 aa, Alternaria alternata
DITYVATATLPNYCR

SEQ ID NO: 135 - protein, 14 aa, Alternaria alternata
DITYVATATLPNYC

SEQ ID NO: 136 - protein, 14 aa, Alternaria alternata
ITYVATATLPNYCR

SEQ ID NO: 137 - protein, 13 aa, Alternaria alternata
ITYVATATLPNYC

SEQ ID NO: 138 - protein, 13 aa, Alternaria alternata
TYVATATLPNYCR

SEQ ID NO: 139 - protein, 12 aa, Alternaria alternata
TYVATATLPNYC

SEQ ID NO: 140 - protein, 12 aa, Alternaria alternata
ISEFYGRKPEGT

SEQ ID NO: 141 - protein, 11 aa, Alternaria alternata
SEFYGRKPEGT

SEQ ID NO: 142 - protein, 10 aa, Alternaria alternata
EFYGRKPEGT

SEQ ID NO: 143 - protein, 12 aa, Alternaria alternata
FYGRKPEGTYYN

SEQ ID NO: 144 - protein, 11 aa, Alternaria alternata
FYGRKPEGTYY

SEQ ID NO: 145 - protein, 10 aa, Alternaria alternata
FYGRKPEGTY

SEQ ID NO: 146 - protein, 14 aa, Alternaria alternata
ISEFYGRKPEGTYY

SEQ ID NO: 147 - protein, 13 aa, Alternaria alternata
ISEFYGRKPEGTY

SEQ ID NO: 148 - protein, 14 aa, Alternaria alternata
SEFYGRKPEGTYYN

SEQ ID NO: 149 - protein, 13 aa, Alternaria alternata
SEFYGRKPEGTYY

SEQ ID NO: 150 - protein, 12 aa, Alternaria alternata
SEFYGRKPEGTY

SEQ ID NO: 151 - protein, 13 aa, Alternaria alternata
EFYGRKPEGTYYN

SEQ ID NO: 152 - protein, 12 aa, Alternaria alternata
EFYGRKPEGTYY

SEQ ID NO: 153 - protein, 11 aa, Alternaria alternata
EFYGRKPEGTY

SEQ ID NO: 154 - protein, 9 aa, Alternaria alternata
FYGRKPEGT

SEQ ID NO: 155 - protein, 12 aa, Alternaria alternata
SEFYGRKPEGTY

SEQ ID NO: 156 - protein, 11 aa, Alternaria alternata
EFYGRKPEGTY

SEQ ID NO: 157 - protein, 10 aa, Alternaria alternata
FYGRKPEGTY

SEQ ID NO: 158 - protein, 12 aa, Alternaria alternata
YGRKPEGTYYNS

SEQ ID NO: 159 - protein, 11 aa, Alternaria alternata
YGRKPEGTYYN

SEQ ID NO: 160 - protein, 10 aa, Alternaria alternata
YGRKPEGTYY

SEQ ID NO: 161 - protein, 14 aa, Alternaria alternata
SEFYGRKPEGTYYN

SEQ ID NO: 162 - protein, 13 aa, Alternaria alternata
SEFYGRKPEGTYY

SEQ ID NO: 163 - protein, 14 aa, Alternaria alternata
EFYGRKPEGTYYNS

SEQ ID NO: 164 - protein, 13 aa, Alternaria alternata
EFYGRKPEGTYYN

SEQ ID NO: 165 - protein, 12 aa, Alternaria alternata
EFYGRKPEGTYY

SEQ ID NO: 166 - protein, 13 aa, Alternaria alternata
FYGRKPEGTYYNS

SEQ ID NO: 167 - protein, 12 aa, Alternaria alternata
FYGRKPEGTYYN

SEQ ID NO: 168 - protein, 11 aa, Alternaria alternata
FYGRKPEGTYY

SEQ ID NO: 169 - protein, 9 aa, Alternaria alternata
YGRKPEGTY

SEQ ID NO: 170 - protein, 12 aa, Alternaria alternata
AAYLLLGLGGNT

SEQ ID NO: 171 - protein, 11 aa, Alternaria alternata
AYLLLGLGGNT

SEQ ID NO: 172 - protein, 10 aa, Alternaria alternata
YLLLGLGGNT

SEQ ID NO: 173 - protein, 12 aa, Alternaria alternata
LLLGLGGNTSPS

SEQ ID NO: 174 - protein, 11 aa, Alternaria alternata
LLLGLGGNTSP

SEQ ID NO: 175 - protein, 10 aa, Alternaria alternata
LLLGLGGNTS

SEQ ID NO: 176 - protein, 14 aa, Alternaria alternata
AAYLLLGLGGNTSP

SEQ ID NO: 177 - protein, 13 aa, Alternaria alternata
AAYLLLGLGGNTS

SEQ ID NO: 178 - protein, 14 aa, Alternaria alternata
AYLLLGLGGNTSPS

SEQ ID NO: 179 - protein, 13 aa, Alternaria alternata
AYLLLGLGGNTSP

SEQ ID NO: 180 - protein, 12 aa, Alternaria alternata
AYLLLGLGGNTS

SEQ ID NO: 181 - protein, 13 aa, Alternaria alternata
YLLLGLGGNTSPS

SEQ ID NO: 182 - protein, 12 aa, Alternaria alternata
YLLLGLGGNTSP

SEQ ID NO: 183 - protein, 11 aa, Alternaria alternata
YLLLGLGGNTS

SEQ ID NO: 184 - protein, 9 aa, Alternaria alternata
LLLGLGGNT

SEQ ID NO: 185 - protein, 9 aa, Alternaria alternata
YYNSLGFNI

SEQ ID NO: 186 - protein, 9 aa, Alternaria alternata
FNIKATNGG

SEQ ID NO: 187 - protein, 9 aa, Alternaria alternata
IKATNGGTL

SEQ ID NO: 188 - protein, 9 aa, Alternaria alternata
VATATLPNY

SEQ ID NO: 189 - protein, 9 aa, Alternaria alternata
YVATATLPN

SEQ ID NO: 190 - protein, 9 aa, Alternaria alternata
YITLVTLPK

SEQ ID NO: 191 - protein, 9 aa, Alternaria alternata
ITLVTLPKS

SEQ ID NO: 192 - protein, 9 aa, Artificial sequence
  Synthetic sequence Peptide derivative
VYQKLKALA

SEQ ID NO: 193 - protein, 9 aa, Artificial sequence
  Synthetic sequence Peptide derivative
YQKLKALAK

SEQ ID NO: 194 - protein, 9 aa, Artificial sequence
  Synthetic sequence Peptide derivative
KLKALAKKT

SEQ ID NO: 195 - protein, 9 aa, Artificial sequence
  Synthetic sequence Peptide derivative
LKALAKKTY

SEQ ID NO: 196 - protein, 9 aa, Artificial sequence
  Synthetic sequence Peptide derivative
FGAGWGVMV

SEQ ID NO: 197 - protein, 9 aa, Artificial sequence
  Synthetic sequence Peptide derivative
WGVMVSHRS

SEQ ID NO: 198 - protein, 9 aa, Artificial sequence
  Synthetic sequence Peptide derivative
WGVLVSHRS

SEQ ID NO: 199 - protein, 9 aa, Artificial sequence
  Synthetic sequence Peptide derivative
GVMVSHRSG

SEQ ID NO: 200 - protein, 9 aa, Artificial sequence
  Synthetic sequence Peptide derivative
VMVSHRSGE

SEQ ID NO: 201 - protein, 9 aa, Artificial sequence
  Synthetic sequence Peptide derivative
MVSHRSGET

SEQ ID NO: 202 - protein, 9 aa, Alternaria alternata
YVWKISEFY

SEQ ID NO: 203 - protein, 9 aa, Alternaria alternata
LLLKQKVSD

SEQ ID NO: 204 - protein, 9 aa, Alternaria alternata
LLKQKVSDD

SEQ ID NO: 205 - protein, 9 aa, Artificial sequence
  Synthetic sequence Peptide derivative
VVLVAYFAA

SEQ ID NO: 206 - protein, 9 aa, Artificial sequence
  Synthetic sequence Peptide derivative
VVGRQILKS

SEQ ID NO: 207 - protein, 9 aa, Artificial sequence
  Synthetic sequence Peptide derivative
VVGRQIMKS

SEQ ID NO: 208 - protein, 9 aa, Alternaria alternata
MQFTTIASL

SEQ ID NO: 209 - protein, 9 aa, Alternaria alternata
FTTIASLFA

SEQ ID NO: 210 - protein, 9 aa, Alternaria alternata
IASLFAAAG

SEQ ID NO: 211 - protein, 9 aa, Alternaria alternata
LFAAAGLAA

SEQ ID NO: 212 - protein, 9 aa, Alternaria alternata
WKISEFYGR

SEQ ID NO: 213 - protein, 9 aa, Alternaria alternata
MKHLAAYLL

SEQ ID NO: 214 - protein, 9 aa, Artificial sequence
  Synthetic sequence Peptide derivative
LKHLAAYLL

SEQ ID NO: 215 - protein, 266 aa, Homo sapiens
MVCLKLPGGSCMAALTVTLMVLSSPLALAGDTQPRFLWQGKYKCHFFNGTERVQFLERLFYNQEEFVRFDSDVGEYRAVTELGRPVAESWNSQKDILEDRRGQVDTVCRHNYGVGESFTVQRRVHPEVTVYPAKTQPLQHHNLLVCSVSGFYPGSIEVRWFRNGQEEKAGVVSTGLIQNGDWTFQTLVMLETVPRSGEVYTCQVEHPSVMSPLTVEWRARSESAQSKMLSGVGGFVLGLLFLGAGLFIYFRNQKGHSGLQPTGFLS

SEQ ID NO: 216 - protein, 266 aa, Homo sapiens
MVCLKLPGGSCMTALTVTLMVLSSPLALSGDTRPRFLWQPKRECHFFNGTERVRFLDRYFYNQEESVRFDSDVGEFRAVTELGRPDAEYWNSQKDILEQARAAVDTYCRHNYGVVESFTVQRRVQPKVTVYPSKTQPLQHHNLLVCSVSGFYPGSIEVRWFLNGQEEKAGMVSTGLIQNGDWTFQTLVMLETVPRSGEVYTCQVEHPSVTSPLTVEWRARSESAQSKMLSGVGGFVLGLLFLGAGLFIYFRNQKGHSGLQPTGFLS

SEQ ID NO: 217 - protein, 24 aa, Homo sapiens [sequence omitted]

SEQ ID NO: 218 - protein, 266 aa, Homo sapiens [sequence omitted]

SEQ ID NO: 219 - protein, 266 aa, Homo sapiens
MVCLKLPGGSCMTALTVTLMVLSSPLALAGDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRARSESAQSKMLSGVGGFVLGLLFLGAGLFIYFRNQKGHSGLQPTGFLS

SEQ ID NO: 220 - protein, 266 aa, Homo sapiens [sequence omitted]

SEQ ID NO: 221 - protein, 24 aa, Homo sapiens [sequence omitted]

SEQ ID NO: 222 - protein, 224 aa, Homo sapiens
ECHFFNGTERVRFLDRYFHNQEENVRFDSDVGEFRAVTELGRPDAEYWNSQKDILEDERAAVDTYCRHNYGVGESFTVQRRVHPKVTVYPSKTQPLQHHNLLVCSVSGFYPGSIEVRWFRNGQEEKTGVVSTGLIHNGDWTFQTLVMLETVPRSGEVYTCQVEHPSVTSPLTVEWRARSESAQSKMLSGVGGFVLGLLFLGAGLFIYFRNQKGHSGLQPRGFLS

SEQ ID NO: 223 - protein, 266 aa, Homo sapiens [sequence omitted]

SEQ ID NO: 224 - protein, 157 aa, Alternaria alternata
MQFTTIASLFAAAGLAAAAPLESRQDTASCPVTTEGDYVWKISEFYGRKPEGTYYNSLGFNIKATNGGTLDFTCSAQADKLEDHKWYSCGENSFMDFSFDSDRSGLLLKQKVSDDITYVATATLPNYCRAGGNGPKDFVCQGVADAYITLVTLPKSS

SEQ ID NO: 225 - protein, 113 aa, Alternaria alternata
MKHLAAYLLLGLGGNTSPSAADVKAVLESVGIEADSDRLDKLISELEGKDINELIASGSEKLASVPSGGAGGAAASGGAAAAGGSAQAEAAPEAAKEEEKEESDEDMGFGLFD

SEQ ID NO: 226 - protein, 157 aa, Alternaria alternata; identical to SEQ ID NO: 224

SEQ ID NO: 227 - protein, 113 aa, Alternaria alternata; identical to SEQ ID NO: 225

(19) United States
(12) Patent Application Publication
(10) Pub. No.: US 2016/0130311 A1
(43) Pub. Date: May 12, 2016

(54) T CELL EPITOPES DERIVED FROM ALT A 1 OR ALT A 5 FOR THE TREATMENT OF ALTERNARIA ALTERNATA ALLERGY

(71) Applicants: Maria R. DIAZ-TORRES, El Sauzal, Tenerife (ES); Nigel S. DUNN-COLEMAN, El Sauzal, Tenerife (ES); Brian S. MILLER, Elgin, IL (US); ALERGENETICA SL, San Cristobal de Laguna (ES)

(72) Inventors: Brian S. Miller, Elgin, IL (US); Laura Miller, Elgin, IL (US); Maria R. Diaz-Torres, El Sauzal, Tenerife (ES); Nigel S. Dunn-Coleman, El Sauzal, Tenerife (ES); Laura Claverie-Diaz, Santa Cruz de Tenerife (ES)

(73) Assignee: Maria R. Diaz-Torres, El Sauzal, Tenerife (ES)

(21) Appl. No.: 14/896,287

(22) PCT Filed: Jun. 4, 2014
(86) PCT No.: PCT/EP2014/061641

§ 371 Date: Dec. 4, 2015

Related U.S. Application Data
(60) Provisional application No. 61/831,201, filed on Jun. 5, 2013.

(51) Int. Cl. C07K 14/37; C07K 7/08; G01N 33/68; C07K 7/06; A61K 39/00; G01N 33/50

(52) U.S. Cl. CPC C07K 14/37; A61K 39/0002; G01N 33/505; G01N 33/6893; C07K 7/06; C07K 7/08

(57) ABSTRACT

The application discloses peptides capable of preventing or treating fungal disease, including fungal allergy disease.

### FIELD OF THE INVENTION

The present invention relates to peptides capable of preventing or treating fungal disease and particularly, although not exclusively, to peptides useful in the prevention or treatment of fungal allergy disease.

### BACKGROUND TO THE INVENTION

Fungal allergy is a common condition that significantly compromises the quality of life of many patients (1, 2). Asthma and rhinitis are common clinical symptoms from exposure to fungal spores and there is increasing evidence of a connection between fungal allergy and the development and persistence of moderate to severe life-threatening asthma (3). Alternaria alternata is one of the most important fungi in respiratory allergic disease worldwide. Airborne exposure of A. alternata can first cause sensitization that may later result in the development of a fungal allergic disease. Surveys in Europe, USA, Australia and New Zealand have shown significant sensitization to A. alternata in allergic and general populations. In allergic asthmatic populations, sensitization rates for A. alternata can vary from 1.7 to 28.2% (4, 5). In a standardized general population, sensitization rates vary between 0.2 to 14.4% (6). In Spain, sensitization rates have been reported up to 18.3% in allergic populations (7). A. alternata is primarily found in outdoor environments particularly in soil, plants and air, but is also found in indoor environments such as house dust, carpets, and textiles (8).

The avoidance of spores, use of medications such as antihistamines to treat allergy symptoms, and/or use of extract immunotherapy are the only current options for alleviating symptoms induced by Alternaria allergy. Alternaria allergy has been successfully treated with fungal extract immunotherapy but requires long term administration and may have potential side effects including anaphylaxis (9-12). In addition, there may be some concern with treating patients with fungal extracts that contain potentially harmful, mutagenic mycotoxins (13). Therefore, efforts to develop an effective and safer immunotherapeutic approach to treat Alternaria allergy and perhaps other fungal allergies are needed. The use of peptides containing T cell epitopes of allergens of interest can be used for immunotherapy (14). Since these peptide fragments are small enough in length they do not cross-link allergen specific IgE on mast cells and basophils, but provide immunogenicity (15). It has been clearly demonstrated that peptides derived from the major allergens associated with specific allergies have been used for immunotherapy to desensitize patients allergic to cat (16) and bee venom (17).

A principal feature of MHC molecules is their allelic polymorphism, at least 707 class II molecules are known. MHC alleles have arisen under evolutionary pressure resulting in geographical diversity. Any poly-epitope vaccine targeting the whole population would need to bind a range of HLA molecules. MHC polymorphism thus greatly complicates epitope-based vaccine development, particularly in regard to population coverage (Doytchinova and Flower. J. Immunol. 2005. 174:7085-7095).

The Alt a 1 allergen from A. alternata is the major allergen in Alternaria allergic patients with Alt a 1 specific IgE found in >90% of allergic populations (7, 18) and thus provides a target for development of specific peptide immunotherapy.

Some peptides containing T cell epitopes are described in WO2012/038540.

### SUMMARY OF THE INVENTION

The inventors have identified peptides and peptide combinations proposed to be useful in immunotherapy.

The peptides are preferably T-cell epitopes capable of binding human or animal HLA-DR molecules and stimulating an immune response. The peptides are preferably T-cell epitopes identified from Alternaria alternata proteins Alt a 1 or Alt a 5.

Modified peptides are also provided in which the wild type fungal peptide epitope amino acid sequence has been modified but still retains its ability to stimulate an immune response.

Accordingly, the present invention provides therapeutic compositions and methods for treating disease conditions in humans and animals associated with an antigen specific immune response by the human or animal to an antigen such as a protein antigen, preferably Alt a 1 or Alt a 5.

In one aspect of the present invention a combination of peptides is provided, the combination being proposed as useful in a method of medical treatment, e.g. immunotherapy.

The inventors have identified seven peptides which are T-cell epitopes identified from Alternaria alternata protein Alt a 1. The seven peptides form a pool or panel from which combinations of the seven peptides can be provided which activate T-cells in a significant proportion of the Alternaria sensitised human population (preferably the Alt a 1 sensitised population). As such, combinations of two or more of such peptides (or their variants and derivatives) can be provided, thereby providing a single immunotherapy treatment for a wide-range of the Alternaria sensitised patient population (preferably the Alt a 1 sensitised population). Combinations include two or more of the seven peptides (or a variant or derivative of a respective peptide) in any combination. In some embodiments no additional peptides beyond those of the pool of seven (optionally including their derivatives ad variants) are included. In some other embodiments an additional peptide(s) from outside the pool may be included in the combination.

As such, combinations contain at least two peptides, each of said at least two peptides selected from a different one of the numbered groups (i) to (vii) given below wherein a peptide consists of or comprises the amino acid sequence defined by the respective SEQ ID NO.
(i) SEQ ID NO: 2, SEQ ID NOs: 27-41
(ii) SEQ ID NO: 4, SEQ ID NOs: 42-56
(iii) SEQ ID NO: 5, SEQ ID NOs: 57-71
(iv) SEQ ID NO: 11, SEQ ID NOs: 72-86
(v) SEQ ID NO: 12, SEQ ID NOs: 87-101
(vi) SEQ ID NO: 20, SEQ ID NOs: 102-116
(vii) SEQ ID NO: 21, SEQ ID NOs: 117-139

In some embodiments the combination may contain three, four, five, six or seven peptides each said peptide selected from a different one of the numbered groups (i) to (vii). For example, a combination of at least two peptides may comprise one peptide from group (i) and one peptide from group (iv) In another example a combination of at least three peptides may comprise one peptide from group (i), one peptide from group (iii) and one peptide from group (vii).

The combinations may contain additional agents, carriers, diluents or excipients. An additional agent may be a further peptide from one of groups (i) to (vii) (e.g. so that two peptides from group (i) are present in the combination) or a peptide not included in one of groups (i) to (vii). For example, the combination may comprise 2, 3, 4, 5, 6, 7, 8, 9, 10, 11, 12, 13, 14, 15, 16, 17, 18, 19, 20, 21, 22, 23, 24, 25, 26, 27, 28, 29 or 30 peptides, in which at least two (optionally three, four, five, six or seven) of the peptides are selected from two (optionally three, four, five, six or seven respectively) different groups (i) to (vii) above.

In some embodiments a combination contains no more than three (preferably no more than two or one) peptide(s) from a numbered group above. In one embodiment a combination contains no more than one peptide from a numbered group above.

The combinations may have a maximum of any one of 2, 3, 4, 5, 6, 7, 8, 9, 10, 11, 12, 13, 14, 15, 16, 17, 18, 19, 20, 21, 22, 23, 24, 25, 26, 27, 28, 29 or 30 different peptides. Where there are 7 or less different peptides, each one may be selected from one of groups (i) to (vii).

In some preferred embodiments at least one of the peptides is selected from group (iii).

In some preferred embodiments at least one peptide is selected from group (iii) and at least one peptide is selected from group (i). As such, in some embodiments the combination may contain only two peptides selected from groups (i) to (vii), one selected from group (iii) and one from group (i). In some embodiments other peptides not from groups (i) to (vii) may optionally be included in the combination or the combination may exclude such other peptides.

In some preferred embodiments at least one peptide is selected from group (iii), at least one peptide is selected from group (ii) and at least one peptide is selected from group (iv). As such, in some embodiments the combination may contain only three peptides selected from groups (i) to (vii), one selected from group (iii), one from group (ii) and one from group (iv). In some embodiments other peptides not from groups (i) to (vii) may optionally be included in the combination or the combination may exclude such other peptides.

In some preferred embodiments at least one peptide is selected from group (iii), at least one peptide is selected from group (ii) and at least one peptide is selected from group (v). As such, in some embodiments the combination may contain only three peptides selected from groups (i) to (vii), one selected from group (iii), one from group (ii) and one from group (v). In some embodiments other peptides from groups (i) to (vii) may optionally be included in the combination. In some embodiments other peptides not from groups (i) to (vii) may optionally be included in the combination or the combination may exclude such other peptides.

In some preferred embodiments at least one peptide is selected from group (iii), at least one peptide is selected from group (ii) and at least one peptide is selected from group (vi). As such, in some embodiments the combination may contain only three peptides selected from groups (i) to (vii), one selected from group (iii), one from group (ii) and one from group (vi). In some embodiments other peptides from groups (i) to (vii) may optionally be included in the combination. In some embodiments other peptides not from groups (i) to (vii) may optionally be included in the combination or the combination may exclude such other peptides.

In some preferred embodiments at least one peptide is selected from group (iii), at least one peptide is selected from group (iv) and at least one peptide is selected from group (vii). As such, in some embodiments the combination may contain only three peptides selected from groups (i) to (vii), one selected from group (iii), one from group (iv) and one from group (vii). In some embodiments other peptides from groups (i) to (vii) may optionally be included in the combination. In some embodiments other peptides not from groups (i) to (vii) may optionally be included in the combination or the combination may exclude such other peptides.

In some preferred embodiments at least one peptide is selected from each of groups (i) to (vii). As such, in some embodiments the combination may contain only seven peptides. In some embodiments other peptides from groups (i) to (vii) may optionally be included in the combination. In some embodiments other peptides not from groups (i) to (vii) may optionally be included in the combination or the combination may exclude such other peptides.

In some preferred embodiments a combination of 2, 3 or 4 peptides is provided wherein at least one peptide is selected from two, three or four of groups (i), (ii), (iii) and (iv) respectively. As such, in some embodiments the combination may contain only 2, 3, or 4 peptides selected from one of groups (i) to (iv), a maximum of one selected from each said group. In some embodiments other peptides from groups (i) to (vii) may optionally be included in the combination. In some embodiments other peptides not from groups (i) to (vii) may optionally be included in the combination or the combination may exclude such other peptides.

In some embodiments a peptide from groups (v) and/or (vi) and/or (vii) is not included in the combination.

Additional peptides that may be included in a combination include any one of SEQ ID NOs 1, 3, 6-10, 13-19 and 22-25 (FIG. 8) or a peptide variant containing the 9mer core sequence (underlined in FIG. 8), or a peptide from group (b), (c) or (d).

The peptides combinations of the present invention may be provided in a number of ways. For example, single compositions may be provided containing all of the respective peptides of the combination. This may be in the form of a pharmaceutical composition or medicament. Alternatively, peptides of the combination may be divided into one or more separate compositions which are provided for use in combination in a method of medical treatment, e.g. by simultaneous, sequential or separate administration.

Accordingly, in one aspect of the present invention a composition or preparation is provided comprising at least two peptides, each of said at least two peptides selected From a different one of groups (i) to (vii) wherein a peptide consists of or comprises the amino acid sequence defined by the respective SEQ ID NO, and wherein each peptide has an amino acid length of from 8 to 50 amino acids
(i) SEQ ID NO: 2, SEQ ID NOs: 27-41
(ii) SEQ ID NO: 4, SEQ ID NOs: 42-56
(iii) SEQ ID NO: 5, SEQ ID NOs: 57-71
(iv) SEQ ID NO: 11, SEQ ID NOs: 72-86
(v) SEQ ID NO: 12, SEQ ID NOs: 87-101
(vi) SEQ ID NO: 20, SEQ ID NOs: 102-116
(vii) SEQ ID NO: 21, SEQ ID NOs: 117-139.

In some embodiments each peptide has a maximum length of 15 amino acids and a minimum length of 9 amino acids. In some embodiments the composition has at least one peptide from group (iii). In some embodiments the composition has at least one peptide from each of groups (iii) and (i). In some embodiments the composition has at least one peptide from each of groups (iii), (ii) and (iv). In some embodiments the composition has at least one peptide from each of groups (iii), (ii) and (v). In some embodiments the composition has at least one peptide from each of groups (iii), (ii) and (vi). In some embodiments the composition has at least one peptide from each of groups (iii), (iv) and (vii). In some embodiments the composition has at least three, four, five, six or seven peptides, wherein each peptide is from a different one of groups (i) to (vii). In some embodiments the composition has seven peptides, wherein each peptide is from a different one of groups (i) to (vii).

In another aspect of the present invention a peptide is provided for use in a method for the prevention or treatment of disease wherein the peptide is selected from one of groups (i) to (vii), the method comprising simultaneous, sequential or separate administration of at least two peptides, each of said at least two peptides selected from a different one of groups (i) to (vii), wherein each peptide consists of or comprises the amino acid sequence defined by the respective SEQ ID NO, and wherein each peptide has an amino acid length of from 8 to 50 amino acids
(i) SEQ ID NO: 2, SEQ ID NOs: 27-41
(ii) SEQ ID NO: 4, SEQ ID NOs: 42-56
(iii) SEQ ID NO: 5, SEQ ID NOs: 57-71
(iv) SEQ ID NO: 11, SEQ ID NOs: 72-86
(v) SEQ ID NO: 12, SEQ ID NOs: 87-101
(vi) SEQ ID NO: 20, SEQ ID NOs: 102-116
(vii) SEQ ID NO: 21, SEQ ID NOs: 117-139.

In another aspect of the present invention the use of a peptide in the manufacture of a medicament for the prevention or treatment of disease is provided wherein the peptide is selected from one of groups (i) to (vii), and the method of prevention or treatment comprises simultaneous, sequential or separate administration of at least two peptides, each of said at least two peptides selected from a different one of groups (i) to (vii), wherein each peptide consists of or comprises the amino acid sequence defined by the respective SEQ ID NO, and wherein each peptide has an amino acid length of from 8 to 50 amino acids
(i) SEQ ID NO: 2, SEQ ID NOs: 27-41
(ii) SEQ ID NO: 4, SEQ ID NOs: 42-56
(iii) SEQ ID NO: 5, SEQ ID NOs: 57-71
(iv) SEQ ID NO: 11, SEQ ID NOs: 72-86
(v) SEQ ID NO: 12, SEQ ID NOs: 87-101
(vi) SEQ ID NO: 20, SEQ ID NOs: 102-116
(vii) SEQ ID NO: 21, SEQ ID NOs: 117-139.

In another aspect of the present invention peptides are provided for use in a method for the prevention or treatment of disease, the method comprising simultaneous, sequential or separate administration of the peptides, wherein the peptides comprise at least two peptides, each of said at least two peptides selected from a different one of groups (i) to (vii) wherein a peptide consists of or comprises the amino acid sequence defined by the respective SEQ ID NO, and wherein each peptide has an amino acid length of from 8 to 50 amino acids
(i) SEQ ID NO: 2, SEQ ID NOs: 27-41
(ii) SEQ ID NO: 4, SEQ ID NOs: 42-56
(iii) SEQ ID NO: 5, SEQ ID NOs: 57-71
(iv) SEQ ID NO: 11, SEQ ID NOs: 72-86
(v) SEQ ID NO: 12, SEQ ID NOs: 87-101
(vi) SEQ ID NO: 20, SEQ ID NOs: 102-116
(vii) SEQ ID NO: 21, SEQ ID NOs: 117-139.

In another aspect of the present invention the use of at least two peptides in the manufacture of a medicament for the prevention or treatment of disease is provided, wherein the peptides comprise at least two peptides, each of said at least two peptides selected from a different one of groups (i) to (vii) wherein a peptide consists of or comprises the amino acid sequence defined by the respective SEQ ID NO, and wherein each peptide has an amino acid length of from 8 to 50 amino acids
(i) SEQ ID NO: 2, SEQ ID NOs: 27-41
(ii) SEQ ID NO: 4, SEQ ID NOs: 42-56
(iii) SEQ ID NO: 5, SEQ ID NOs: 57-71
(iv) SEQ ID NO: 11, SEQ ID NOs: 72-86
(v) SEQ ID NO: 12, SEQ ID NOs: 87-101
(vi) SEQ ID NO: 20, SEQ ID NOs: 102-116
(vii) SEQ ID NO: 21, SEQ ID NOs: 117-139.

In another aspect of the present invention a method of treating or preventing a disease in a patient in need of treatment thereof is provided, the method comprising administering to the patient a therapeutically effective amount of at least two peptides, each of said at least two peptides selected from a different one of groups (i) to (vii) wherein a peptide consists of or comprises the amino acid sequence defined by the respective SEQ ID NO, and wherein each peptide has an amino acid length of from 8 to 50 amino acids
(i) SEQ ID NO: 2, SEQ ID NOs: 27-41
(ii) SEQ ID NO: 4, SEQ ID NOs: 42-56
(iii) SEQ ID NO: 5, SEQ ID NOs: 57-71
(iv) SEQ ID NO: 11, SEQ ID NOs: 72-86
(v) SEQ ID NO: 12, SEQ ID NOs: 87-101
(vi) SEQ ID NO: 20, SEQ ID NOs: 102-116
(vii) SEQ ID NO: 21, SEQ ID NOs: 117-139.

In some embodiments the or each peptide has a maximum length of 15 amino acids and a minimum length of 9 amino acids.

In some embodiments at least one peptide is from group (iii). In some embodiments at least one peptide is from each of groups (iii) and (i). In some embodiments at least one peptide is from each of groups (iii), (ii) and (iv). In some embodiments at least one peptide is from each of groups (iii), (ii) and (v). In some embodiments at least one peptide is from each of groups (iii), (ii) and (vi). In some embodiments at least one peptide is from each of groups (iii), (iv) and (vii).

In some embodiments at least three, four, five, six or seven peptides are administered, and each said peptide is preferably from a different one of groups (i) to (vii). In some embodiments seven peptides are administered, and each peptide is preferably from a different one of groups (i) to (vii).

In some embodiments at least two of the peptides are administered in a combined preparation. Optionally, this may be any one of at least three, four, five, six or seven of the peptides.

In some embodiments the disease is an allergic disease, optionally chosen from fungal allergy, fungal asthma, fungal infection, SAFS, ABPA, or Aspergillosis or an allergic disease caused by Alt a 1 or Alt a 5.

In another aspect of the present invention a method for the production of a pharmaceutical composition or medicament is provided, the method comprising providing at least two peptides (optionally one of at least three, four, five, six or seven), each of said at least two peptides (or three, four, five, six or seven) selected from a different one of groups (i) to (vii) wherein a peptide consists of or comprises the amino acid sequence defined by the respective SEQ ID NO, and wherein each peptide has an amino acid length of from 8 to 50 amino acids
(i) SEQ ID NO: 2, SEQ ID NOs: 27-41
(ii) SEQ ID NO: 4, SEQ ID NOs: 42-56
(iii) SEQ ID NO: 5, SEQ ID NOs: 57-71
(iv) SEQ ID NO: 11, SEQ ID NOs: 72-86
(v) SEQ ID NO: 12, SEQ ID NOs: 87-101
(vi) SEQ ID NO: 20, SEQ ID NOs: 102-116
(vii) SEQ ID NO: 21, SEQ ID NOs: 117-139,

and mixing the at least two (or three, four, five, six or seven) peptides with a pharmaceutically acceptable carrier, adjuvant or diluent.

In another aspect of the present invention novel peptides are provided, which are T-cell epitopes identified from Alternaria alternata protein Alt a 1 and Alt a 5. Whilst these may be provided as part of the combinations described above, they may also be provided as isolated peptides, and provide the basis of an immunotherapy treatment as discrete single active agents.

Three such peptides have been identified from Alt a 1, being represented by groups:
(a) SEQ ID NO: 2, SEQ ID NOs: 27-41
(b) SEQ ID NO: 8, SEQ ID NOs: 140-154
(c) SEQ ID NO: 9, SEQ ID NOs: 155-169

Optionally, Group (a) excludes one or more, or all, of SEQ ID NOs: 27, 28, 29, 30, 32, 34, 37, 40 and/or 41. Therefore, in some embodiments Group (a) comprises or consists of one or more, or all, of SEQ ID NOs: 2, 31, 33, 35, 36, 38 and 39.

One such peptide has been identified from Alt a 5, being represent by group:
(d) SEQ ID NO: 26, SEQ ID NOs: 170-184.

As such, in one aspect of the present invention a peptide is provided, the peptide being chosen from a peptide of group (a).

In another aspect of the present invention a peptide is provided, the peptide being chosen from a peptide of group (b).

In another aspect of the present invention a peptide is provided, the peptide being chosen from a peptide of group (c).

In another aspect of the present invention a peptide is provided, the peptide being chosen from a peptide of group (d).

Accordingly, in another aspect of the present invention a peptide is provided, the peptide consisting of or comprising the amino acid sequence of one of
(a) SEQ ID NO: 2, SEQ ID NOs: 31, 33, 35, 36, 38, 39
(b) SEQ ID NO: 8, SEQ ID NOs: 140-154
(c) SEQ ID NO: 9, SEQ ID NOs: 155-169
(d) SEQ ID NO: 26, SEQ ID NOs: 170-184

or a peptide having a contiguous amino acid sequence having at least 70% sequence identity to the amino acid sequence of one of said SEQ ID NOs, wherein the peptide has an amino acid length of from 8 to 50 amino acids, wherein the peptide is not one of SEQ ID NOs: 27, 28, 29, 30, 32, 34, 37, 40 or 41

In some embodiments the degree of sequence identity is chosen from one of 80%, 85%, 90% or 95%.

In some embodiments the peptide has a maximum length of 15 amino acids and a minimum length of 9 amino acids.

In one aspect of the present invention a pharmaceutical composition or medicament is provided comprising a peptide as described above. In some embodiments pharmaceutical composition or medicament may further comprise a pharmaceutically acceptable carrier, adjuvant or diluent. In some embodiments the pharmaceutical composition or medicament is a vaccine.

In one aspect of the present invention the peptide, pharmaceutical composition or medicament is provided for use in the prevention or treatment of disease. In some embodiments the disease is an allergic disease, optionally chosen from fungal allergy, fungal asthma, fungal infection, SAFS, ABPA, Aspergillosis, or an allergic disease caused by or in which the patient is sensitised to Alternaria alternata, and/or to one or both of Alt a 1 or Alt a 5.

In another aspect of the present invention a method of treating or preventing disease in a patient in need of treatment thereof is provided, the method comprising administering to the patient a therapeutically effective amount of a peptide, pharmaceutical composition or medicament as described above.

In a further aspect of the present invention a method for the production of a pharmaceutical composition is provided, the method comprising providing a peptide as described above, and mixing the peptide with a pharmaceutically acceptable carrier, adjuvant or diluent.

In a further aspect of the present invention a nucleic acid encoding a peptide as described herein is provided.

In a further aspect of the present invention a cell having integrated in its genome a nucleic acid encoding a peptide as described herein operably linked to a transcription control nucleic acid sequence is provided.

In a further aspect of the present invention a nucleic acid expression vector having a said nucleic acid operably linked to a transcription control nucleic acid sequence is provided, wherein the vector is configured for expression of a peptide as described herein when transfected into a suitable cell. In a further aspect of the present invention a cell transfected with said nucleic acid expression vector is provided.

In a further aspect of the present invention a method of identifying a peptide that is capable of stimulating an immune response is provided, the method comprising the steps of:
(i) providing a candidate peptide having a contiguous amino acid sequence having at least 70% sequence identity to the amino acid sequence of one of:
   (a) SEQ ID NO: 2, SEQ ID NOs: 31, 33, 35, 36, 38, 39
   (b) SEQ ID NO: 8, SEQ ID NOs: 140-154
   (c) SEQ ID NO: 9, SEQ ID NOs: 155-169
   (d) SEQ ID NO: 26, SEQ ID NOs: 170-184
(ii) testing the ability of the candidate peptide to induce an immune response.

The peptide is preferably not one of SEQ ID NOs: 27, 28, 29, 30, 32, 34, 37, 40 or 41.

In some embodiments step (i) comprises providing a peptide having the amino acid sequence of one of said SEQ ID NOs and chemically modifying the structure of the peptide to provide the candidate peptide. In some embodiments step (ii) comprises contacting the candidate peptide with a population of T cells in vitro and assaying T cell proliferation. Step (ii) may comprise monitoring for production of IL-4 and/or IFNγ.

Description

The inventors have conducted the first study to develop a specific peptide mixture for potential Alternaria immunotherapy. Whilst not wishing to be bound by theory, the inventors hypothesized that in silico prediction of specific T cell epitope binding cores combined with an in vitro MHC binding assay allows a rapid and precise method to identify and produce peptide immunotherapy candidates under conditions of limited patient cell numbers. For peptide confirmation the inventors tested the sensitivity of direct PBMC based IL-4 ELISPOT and the relation of ELISPOT results between disease groups vs. controls to determine peptide promiscuity and population coverage. This strategy produced an Alt a 1 peptide pool for potential peptide immunotherapy with high promiscuity and population coverage.

The inventors analyzed sample sparing methods for the prediction and validation of T-cell epitope containing peptides from the major A. alternata allergen Alt a 1, as well as for the A. alternata allergen Alt a 5, for generation of a peptide immunotherapy mixture of high patient population coverage.

T-cell epitopes were predicted using the ProPred algorithm. The results of T-cell epitope prediction using ProPred were directly analyzed using an in vitro MHC binding assay followed by IL-4 ELISPOT of HLA typed Alternaria allergic patient and control peripheral blood mononuclear cells (PBMCs). Patient and control ELISPOT counts were processed and analyzed to derive cut-off values for peptide population coverage calculations and potential immunotherapy mix determinations.

Seven 15mer peptides were identified which activated T-cells in ≧40% of the Alternaria patient population. Various combinations of the 7 peptides could be recognized by >90% of the patient population and represent a potential pool for immunotherapy. T-cell stimulating activity was correlated with lower peptide hydrophilicity and solubility. Single residue changes to peptide N-termini were sufficient to improve solubility for the majority of insoluble peptides. Other residue substitutions introduced for oxidation stability did not preclude peptides from binding MHC or stimulating multiple subjects. Retrospective analysis showed that NetMHCIIpan predicted peptides in the same four regions as ProPred including the top 7 peptides from the study however, ProPred had a higher overall false positive rate for several alleles.

As such the inventors have been able to identify novel T-cell epitope-based Alt a 1 peptides and combinations of such peptides as candidates for a T-cell targeted fungal-specific immunotherapy for an HLA-diverse population.

The inventors were also able to identify a novel T-cell epitope-based Alt a 5 peptide as a candidate for a T-cell targeted fungal-specific immunotherapy for an HLA-diverse population.

In aspects of the present invention a peptide may consist of or comprises the primary amino acid sequence of a respective SEQ ID NO. As such, the amino acid sequence of the selected SEQ ID NO is preferably included in the peptide as a contiguous amino acid sequence.

In some aspects a peptide has at least 60% amino acid sequence identity to the primary amino acid sequence of a respective SEQ ID NO. More preferably, the degree of sequence identity is one of 65%, 70%, 75%, 80%, 85%, 87%, 90%, 91%, 92%, 93%, 94%, 95%, 96%, 97%, 98%, 99% or 100% identity.

The minimum epitope for HLA DR recognition may be any of 7-11 amino acids in length and is typically a 9-mer epitope. Improved binding may be afforded by including at least one, two or three amino acids at one or both ends of the minimum epitope. Accordingly, peptides are provided as part of the present invention having a core 9-mer amino acid sequence (e.g. SEQ ID NOs:41, 56, 71, 86, 101, 116, 131, 154, 169, 184) as well as an additional one, two, three, four, five, six (or more) amino acids of any type or combination at the N-terminal end, C-terminal end or at both the N- and C-terminal ends of the sequence. For example, a peptide may have a core amino acid sequence of any one of SEQ ID NOS: 41, 56, 71, 86, 101, 116, 131, 154, 169, 184 as well as an additional 1, 2, 3, 4, 5, 6, 7, 8, 9, 10, 11, 12, 13, 14, 15, amino acids at the N-terminal end, C-terminal end or at both the N- and C-terminal ends of the sequence.

The additional amino acids preferably correspond to amino acids from the parent protein amino acid sequence from which the peptide is derived, i.e. the wild-type amino acid sequence of the protein. For example, SEQ ID NOs: 41, 56, 71, 86, 101, 116, 131, 154, 169 are from the Alt a 1 protein (the position of the peptide in the Alt a 1 polypeptide is indicated in FIG. 8). The full length 157 amino acid Alt a 1 sequence can be found in the UniProt database under Accession No. P79085 (reproduced in FIG. 12). SEQ ID NO: 184 is from the Alt a 5 protein (the position of the peptide in the Alt a 5 polypeptide is indicated in FIG. 8). The full length 113 amino acid sequence of Alt a 5 sequence can be found in the UniProt database under Accession No. P42037 (reproduced in FIG. 13).

In some instances, addition of amino acids corresponding to those in the parent protein sequence in this way results in an unstable amino acid, e.g. cysteine (C), occurring at the N- and/or the C-terminal end of the peptide. In such cases, an unstable amino acid may be substituted by a more stable amino acid. For example, a C/V and/or M/L substitution may be made (see, for example, SEQ ID NOs: 120, 121, 21, 123, 125, 126, 128, and 129).

A peptide may have a maximum length of 30 amino acids and a minimum length of 9 amino acids, or a maximum length of 20 amino acids and a minimum length of 11 amino acids, or a maximum length of 15 amino acids and a minimum length of 9 amino acids, or a maximum length of 11 amino acids and a minimum length of 8 amino acids, or a length of 9 or 15 amino acids. Each of the peptides specifically described herein is preferably capable of stimulating an immune response to Alt a 1 or Alt a 5 respectively.

In some embodiments a peptide has a contiguous amino acid sequence having at least 70% sequence identity to the amino acid sequence of a peptide selected from one of groups (i) to (vii), groups (a) to (c) or group (d), wherein the peptide has an amino acid length of from 8 to 50 amino acids.

The degree of sequence identity may be chosen from one of 80%, 85%, 90% or 95%. The peptide may have a maximum length of 30 amino acids and a minimum length of 9 amino acids, or a maximum length of 20 amino acids and a minimum length of 11 amino acids, or a maximum length of 15 amino acids and a minimum length of 9 amino acids, or a maximum length of 11 amino acids and a minimum length of 8 amino acids, or a length of 9 or 15 amino acids. The peptide is preferably capable of stimulating an immune response to Alt a 1 or Alt a 5 respectively.

In some embodiments a peptide is provided comprising the amino acid sequence of a peptide selected from one of groups (i) to (vii), groups (a) to (c) or group (d) or a peptide having a contiguous amino acid sequence having at least 80% sequence identity to the amino acid sequence of a peptide selected from one of groups (i) to (vii), groups (a) to (c) or group (d), wherein the peptide has an amino acid length of from 8 to 50 amino acids.

In one aspect of the present invention a pharmaceutical composition is provided, the pharmaceutical composition comprising a peptide or peptide combination according to any of the aspects and embodiments described herein. The pharmaceutical composition may further comprise a pharmaceutically acceptable carrier, adjuvant or diluent. The pharmaceutical composition may be a vaccine.

In some aspects of the present invention the peptide(s) or peptide combination and/or pharmaceutical compositions are provided for use in the prevention or treatment of disease. The disease may be an allergic disease. The disease may be chosen from fungal allergy, fungal asthma, fungal infection, SAFS (Severe Asthma with Fungal Sensitisation [Denning et al. Eur. Respir. J. 2006. 27: 615-626]), ABPA (Allergic Bronchopulmonary Aspergillosis), or Aspergillosis. The disease may be an allergic disease caused by an Alternaria alternata protein allergen (preferably Alt a 1 or Alt a 5) or by infection of tissue by Alternaria alternata.

In another aspect of the present invention a method of treating or preventing disease in a patient in need of treatment thereof is provided, the method comprising administering to the patient a therapeutically effective amount of a peptide combination or peptide or pharmaceutical composition according to any one of the aspects and embodiments described herein.

In another aspect of the present invention a method for the production of a pharmaceutical composition is provided, the method comprising providing a peptide combination or peptide according to any one of the aspects and embodiments described herein, and mixing the peptide combination or peptide with a pharmaceutically acceptable carrier, adjuvant or diluent.

Methods for the production of a pharmaceutical composition comprising a peptide combination may comprise a step of mixing the two or more peptides to be contained in the pharmaceutical composition. This step may be undertaken prior to or after mixing of one or more of the peptides with a pharmaceutically acceptable carrier, adjuvant or diluent.

In another aspect of the present invention a nucleic acid, preferably an isolated and/or purified nucleic acid, encoding a peptide according to any one of the aspects and embodiments described herein is provided, although preferably a peptide selected from one of groups (a) to (c) or group (d). A cell is also provided, having integrated in its genome a nucleic acid encoding a peptide according to any one of the aspects and embodiments described herein (although preferably a peptide selected from one of groups (a) to (c) or group (d)) operably linked to a transcription control nucleic acid sequence. A nucleic acid expression vector is also provided having a nucleic acid encoding a peptide according to any one of the aspects and embodiments described herein (although preferably a peptide selected from one of groups (a) to (c) or group (d)) operably linked to a transcription control nucleic acid sequence, wherein the vector is configured for expression of a peptide according to any one of the aspects and embodiments described herein (although preferably a peptide selected from one of groups (a) to (c) or group (d)) when transfected into a suitable cell. Accordingly, a cell transfected with the nucleic acid expression vector is also provided.

In another aspect of the present invention a method of identifying a peptide that is capable of stimulating an immune response is provided, the method comprising the steps of:
(i) providing a candidate peptide having a contiguous amino acid sequence having at least 70% sequence identity to the amino acid sequence of a peptide selected from one of groups (a) to (c) or group (d), wherein the peptide has an amino acid length of from 8 to 50 amino acids, and
(ii) testing the ability of the candidate peptide to induce an immune response.

Step (i) may comprise providing a peptide having the sequence of a peptide selected from one of groups (a) to (c) or group (d) and chemically modifying the structure of the peptide to provide the candidate peptide. Step (ii) may comprise contacting the candidate peptide with a population of T cells in vitro and assaying T cell proliferation. Step (ii) may comprise or further comprise monitoring for production of IL-4 and/or IFNγ.

In aspects and embodiments of the present invention a peptide is provided, the peptide comprising or consisting of one of SEQ ID NOs:2, 4, 5, 8, 9, 11, 12, 20, 21, 26, 27-184 set out below. In the sequences shown below, the 9-mer peptide of the corresponding sequence selected from one of SEQ ID NOs: 2, 4, 5, 8, 9, 11, 12, 20, 21, and 26 is shown in bold. As such, in embodiments of the present invention a peptide or Group of peptides may be chosen from one of:

Group (i) or Group (a)

| Peptide | SEQ ID NO: |
|---|---|
| FTTIASLFAAAG | 27 |
| TTIASLFAAAG | 28 |
| TIASLFAAAG | 29 |
| IASLFAAAGLAA | 30 |
| IASLFAAAGLA | 31 |
| IASLFAAAGL | 32 |
| FTTIASLFAAAGLAA | 2 |
| FTTIASLFAAAGLA | 33 |
| FTTIASLFAAAGL | 34 |
| TTIASLFAAAGLAA | 35 |
| TTIASLFAAAGLA | 36 |
| TTIASLFAAAGL | 37 |
| TIASLFAAAGLAA | 38 |
| TIASLFAAAGLA | 39 |
| TIASLFAAAGL | 40 |
| IASLFAAAG | 41 |

SEQ ID NOs:27-41 correspond to SEQ ID NO:2 in which one, two or three additional contiguous amino acids from the Alt a 1 protein sequence are optionally incorporated at the N-terminus, C-terminus and both N- and C-terminus.

In some embodiments group (i) and/or group (a) excludes peptide(s) consisting of or comprising one of SEQ ID NOs: 27, 28, 29, 30, 32, 34, 37, 40 and/or 41 or a peptide(s) having an amino acid sequence that comprises the contiguous amino acid sequence of one of SEQ ID NOs 27, 28, 29, 30, 32, 34, 37, 40 or 41 as part of the amino acid sequence of the peptide.

As such, in some embodiments Group (i) or Group (a) may comprise peptides consisting of the amino acid sequence of SEQ ID NOs 2, 31, 33, 35, 36, 38 and 39.

Group (ii)

| Peptide | SEQ ID NO: |
|---|---|
| ASLFAAAGLAAA | 42 |
| SLFAAAGLAAA | 43 |
| LFAAAGLAAA | 44 |
| FAAAGLAAAAPL | 45 |
| FAAAGLAAAAP | 46 |
| FAAAGLAAAA | 47 |
| ASLFAAAGLAAAAPL | 4 |
| ASLFAAAGLAAAAP | 48 |
| ASLFAAAGLAAAA | 49 |
| SLFAAAGLAAAAPL | 50 |
| SLFAAAGLAAAAP | 51 |
| SLFAAAGLAAAA | 52 |
| LFAAAGLAAAAPL | 53 |
| LFAAAGLAAAAP | 54 |
| LFAAAGLAAAA | 55 |
| FAAAGLAAA | 56 |

SEQ ID NOs:42-56 correspond to SEQ ID NO:4 in which one, two or three additional contiguous amino acids from the Alt a 1 protein sequence are optionally incorporated at the N-terminus, C-terminus and both N- and C-terminus.

Group (iii)

| Peptide | SEQ ID NO: |
|---|---|
| AAGLAAAAPLES | 57 |
| AGLAAAAPLES | 58 |
| GLAAAAPLES | 59 |
| LAAAAPLESRQD | 60 |
| LAAAAPLESRQ | 61 |
| LAAAAPLESR | 62 |
| AAGLAAAAPLESRQD | 5 |
| AAGLAAAAPLESRQ | 63 |
| AAGLAAAAPLESR | 64 |
| AGLAAAAPLESRQD | 65 |
| AGLAAAAPLESRQ | 66 |
| AGLAAAAPLESR | 67 |
| GLAAAAPLESRQD | 68 |
| GLAAAAPLESRQ | 69 |
| GLAAAAPLESR | 70 |
| LAAAAPLES | 71 |

SEQ ID NOs:57-71 correspond to SEQ ID NO:5 in which one, two or three additional contiguous amino acids from the Alt a 1 protein sequence are optionally incorporated at the N-terminus, C-terminus and both N- and C-terminus.

Group (iv)

| Peptide | SEQ ID NO: |
|---|---|
| GTYYNSLGFNIK | 72 |
| TYYNSLGFNIK | 73 |
| YYNSLGFNIK | 74 |
| YNSLGFNIKATN | 75 |
| YNSLGFNIKAT | 76 |
| YNSLGFNIKA | 77 |
| GTYYNSLGFNIKATN | 11 |
| GTYYNSLGFNIKAT | 78 |
| GTYYNSLGFNIKA | 79 |
| TYYNSLGFNIKATN | 80 |
| TYYNSLGFNIKAT | 81 |
| TYYNSLGFNIKA | 82 |
| YYNSLGFNIKATN | 83 |
| YYNSLGFNIKAT | 84 |
| YYNSLGFNIKA | 85 |
| YNSLGFNIK | 86 |

SEQ ID NOs:72-86 correspond to SEQ ID NO:11 in which one, two or three additional contiguous amino acids from the Alt a 1 protein sequence are optionally incorporated at the N-terminus, C-terminus and both N- and C-terminus.

Group (v)

| Peptide | SEQ ID NO: |
|---|---|
| YNSLGFNIKATN | 87 |
| NSLGFNIKATN | 88 |
| SLGFNIKATN | 89 |
| LGFNIKATNGGT | 90 |
| LGFNIKATNGG | 91 |
| LGFNIKATNG | 92 |
| YNSLGFNIKATNGGT | 12 |
| YNSLGFNIKATNGG | 93 |
| YNSLGFNIKATNG | 94 |
| NSLGFNIKATNGGT | 95 |
| NSLGFNIKATNGG | 96 |
| NSLGFNIKATNG | 97 |
| SLGFNIKATNGGT | 98 |
| SLGFNIKATNGG | 99 |
| SLGFNIKATNG | 100 |
| LGFNIKATN | 101 |

SEQ ID NOs:87-101 correspond to SEQ ID NO:12 in which one, two or three additional contiguous amino acids from the Alt a 1 protein sequence are optionally incorporated at the N-terminus, C-terminus and both N- and C-terminus.

Group (vi)

| Peptide | SEQ ID NO: |
|---|---|
| SDDITYVATATL | 102 |
| DDITYVATATL | 103 |
| DITYVATATL | 104 |
| ITYVATATLPNY | 105 |
| ITYVATATLPN | 106 |
| ITYVATATLP | 107 |
| SDDITYVATATLPNY | 20 |
| SDDITYVATATLPN | 108 |
| SDDITYVATATLP | 109 |
| DDITYVATATLPNY | 110 |
| DDITYVATATLPN | 111 |
| DDITYVATATLP | 112 |
| DITYVATATLPNY | 113 |
| DITYVATATLPN | 114 |
| DITYVATATLP | 115 |
| ITYVATATL | 116 |

SEQ ID NOs:102-116 correspond to SEQ ID NO:20 in which one, two or three additional contiguous amino acids from the Alt a 1 protein sequence are optionally incorporated at the N-terminus, C-terminus and both N- and C-terminus.

Group (vii)

| Peptide | SEQ ID NO: |
|---|---|
| DITYVATATLPN | 117 |
| ITYVATATLPN | 118 |
| TYVATATLPN | 119 |
| YVATATLPNYVR | 120* |
| YVATATLPNYV | 121* |
| YVATATLPNY | 122 |
| DITYVATATLPNYVR | 21* |
| DITYVATATLPNYV | 123* |
| DITYVATATLPNY | 124 |
| ITYVATATLPNYVR | 125* |
| ITYVATATLPNYV | 126* |
| ITYVATATLPNY | 127 |
| TYVATATLPNYVR | 128* |
| TYVATATLPNYV | 129* |
| TYVATATLPNY | 130 |
| YVATATLPN | 131 |
| YVATATLPNYCR | 132 |
| YVATATLPNYC | 133 |
| DITYVATATLPNYCR | 134 |
| DITYVATATLPNYC | 135 |
| ITYVATATLPNYCR | 136 |
| ITYVATATLPNYC | 137 |
| TYVATATLPNYCR | 138 |
| TYVATATLPNYC | 139 |

SEQ ID NOs:117-139 correspond to SEQ ID NO:21 in which one, two or three additional contiguous amino acids from the Alt a 1 protein sequence are optionally incorporated at the N-terminus, C-terminus and both N- and C-terminus. SEQ ID NOS: 120, 121, 21, 123, 125, 126, 128, and 129 (indicated by (*)) are Cys/Val substitution variants of wild type SEQ ID NOS: 132-139. In some embodiments, SEQ ID NOs: 120, 121, 21, 123, 125, 126, 128, and 129 are preferred compared to the respective corresponding sequence selected from one of SEQ ID NOs: 132-139.

Group (b)

| Peptide | SEQ ID NO: |
|---|---|
| ISEFYGRKPEGT | 140 |
| SEFYGRKPEGT | 141 |
| EFYGRKPEGT | 142 |
| FYGRKPEGTYYN | 143 |
| FYGRKPEGTYY | 144 |
| FYGRKPEGTY | 145 |
| ISEFYGRKPEGTYYN | 8 |
| ISEFYGRKPEGTYY | 146 |
| ISEFYGRKPEGTY | 147 |
| SEFYGRKPEGTYYN | 148 |
| SEFYGRKPEGTYY | 149 |
| SEFYGRKPEGTY | 150 |
| EFYGRKPEGTYYN | 151 |
| EFYGRKPEGTYY | 152 |
| EFYGRKPEGTY | 153 |
| FYGRKPEGT | 154 |

SEQ ID NOs:140-154 correspond to SEQ ID NO:8 in which one, two or three additional contiguous amino acids from the Alt a 1 protein sequence are optionally incorporated at the N-terminus, C-terminus and both N- and C-terminus.

Group (c)

| Peptide | SEQ ID NO: |
|---|---|
| SEFYGRKPEGTY | 155 |
| EFYGRKPEGTY | 156 |
| FYGRKPEGTY | 157 |
| YGRKPEGTYYNS | 158 |
| YGRKPEGTYYN | 159 |
| YGRKPEGTYY | 160 |
| SEFYGRKPEGTYYNS | 9 |
| SEFYGRKPEGTYYN | 161 |
| SEFYGRKPEGTYY | 162 |
| EFYGRKPEGTYYNS | 163 |
| EFYGRKPEGTYYN | 164 |
| EFYGRKPEGTYY | 165 |
| FYGRKPEGTYYNS | 166 |
| FYGRKPEGTYYN | 167 |
| FYGRKPEGTYY | 168 |
| YGRKPEGTY | 169 |

SEQ ID NOs:155-169 correspond to SEQ ID NO:9 in which one, two or three additional contiguous amino acids from the Alt a 1 protein sequence are optionally incorporated at the N-terminus, C-terminus and both N- and C-terminus.

Group (d)

| Peptide | SEQ ID NO: |
|---|---|
| AAYLLLGLGGNT | 170 |
| AYLLLGLGGNT | 171 |
| YLLLGLGGNT | 172 |
| LLLGLGGNTSPS | 173 |
| LLLGLGGNTSP | 174 |
| LLLGLGGNTS | 175 |
| AAYLLLGLGGNTSPS | 26 |
| AAYLLLGLGGNTSP | 176 |
| AAYLLLGLGGNTS | 177 |
| AYLLLGLGGNTSPS | 178 |
| AYLLLGLGGNTSP | 179 |
| AYLLLGLGGNTS | 180 |
| YLLLGLGGNTSPS | 181 |
| YLLLGLGGNTSP | 182 |
| YLLLGLGGNTS | 183 |
| LLLGLGGNT | 184 |

SEQ ID NOs:170-184 correspond to SEQ ID NO:26 in which one, two or three additional contiguous amino acids from the Alt a 5 protein sequence are optionally incorporated at the N-terminus, C-terminus and both N- and C-terminus.

The invention may optionally exclude peptides comprising or consisting of one or more of the following sequences, or peptides having a contiguous sequence of 7, 8 or 9 amino acids that has one of 90%, 91%, 92%, 93%, 94%, 95%, 96%, 97%, 98%, 99% or 100% sequence identity to one or more of the following sequences:

(SEQ ID NO: 185)
YYNSLGFNI
(SEQ ID NO: 101)
LGFNIKATN
(SEQ ID NO: 186)
FNIKATNGG
(SEQ ID NO: 187)
IKATNGGTL
(SEQ ID NO: 116)
ITYVATATL
(SEQ ID NO: 188)
VATATLPNY
(SEQ ID NO: 189)
YVATATLPN
(SEQ ID NO: 190)
YITLVTLPK
(SEQ ID NO: 191)
ITLVTLPKS
(SEQ ID NO: 192)
VYQKLKALA
(SEQ ID NO: 193)
YQKLKALAK
(SEQ ID NO: 194)
KLKALAKKT
(SEQ ID NO: 195)
LKALAKKTY
(SEQ ID NO: 196)
FGAGWGVMV
(SEQ ID NO: 197)
WGVMVSHRS
(SEQ ID NO: 198)
WGVLVSHRS
(SEQ ID NO: 199)
GVMVSHRSG
(SEQ ID NO: 200)
VMVSHRSGE
(SEQ ID NO: 201)
MVSHRSGET
(SEQ ID NO: 202)
YVWKISEFY
(SEQ ID NO: 203)
LLLKQKVSD
(SEQ ID NO: 204)
LLKQKVSDD
(SEQ ID NO: 205)
WLVAYFAA
(SEQ ID NO: 206)
WGRQILKS
(SEQ ID NO: 207)
WGRQIMKS
(SEQ ID NO: 208)
MQFTTIASL
(SEQ ID NO: 209)
FTTIASLFA
(SEQ ID NO: 210)
IASLFAAAG
(SEQ ID NO: 211)
LFAAAGLAA
(SEQ ID NO: 56)
FAAAGLAAA
(SEQ ID NO: 212)
WKISEFYGR
(SEQ ID NO: 213)
MKHLAAYLL
(SEQ ID NO: 214)
LKHLAAYLL
(SEQ ID NO: 27)
FTTIASLFAAAG
(SEQ ID NO: 28)
TTIASLFAAAG
(SEQ ID NO: 29)
TIASLFAAAG
(SEQ ID NO: 30)
IASLFAAAGLAA
(SEQ ID NO: 32)
IASLFAAAGL
(SEQ ID NO: 34)
FTTIASLFAAAGL
(SEQ ID NO: 37)
TTIASLFAAAGL
(SEQ ID NO: 40)
TIASLFAAAGL

In some embodiments a respective peptide comprises or consists of the amino acid sequence of one of SEQ ID NOs: 2, 4, 5, 8, 9, 11, 12, 20, 21, 26, 27-184 (optionally excluding one or more, or all, of SEQ ID NOs: 27, 28, 29, 30, 32, 34, 37, 40, 41). The amino acid sequence of the selected SEQ ID NO is preferably included in the peptide as a contiguous amino acid sequence.

In some embodiments a respective peptide has at least 60% amino acid sequence identity to one of SEQ ID NOs: 2, 4, 5, 8, 9, 11, 12, 20, 21, 26, 27-184 (optionally excluding one or more, or all, of SEQ ID NOs: 27, 28, 29, 30, 32, 34, 37, 40, 41). More preferably, the degree of sequence identity is one of 65%, 70%, 75%, 80%, 85%, 87%, 90%, 91%, 92%, 93%, 94%, 95%, 96%, 97%, 98%, 99% or 100% identity.

A peptide according to the present invention may have a maximum length of 50 amino acids and less than the full length of the corresponding protein allergen, i.e. Alt a 1 or Alt a 5. More preferably the maximum peptide length is one of 40 amino acids, 30 amino acids, or is chosen from one of 29, 28, 27, 26, 25, 24, 23, 22, 21, 20, 19, 18, 17, 16, 15, 14, 13, 12, 11, 10 or 9 amino acids. For example, a peptide may have a maximum length of one of 20 amino acids, 15 amino acids, 13 amino acids, 11 amino acids or 9 amino acids.

A peptide according to the present invention may have a minimum length of 7 amino acids. Preferably the minimum length is chosen from one of 7, 8, 9, 10, 11, 12, 13, 14, 15, 16, 17, 18, 19 or 20 amino acids. For example, a peptide may have a minimum length of one of 7, 8, 9, 10 or 11 amino acids.

A peptide according to the present invention may have any length between said minimum and maximum. Thus, for example, a peptide may have a length of from 8 to 30, 10 to 25, 12 to 20, 9 to 15 amino acids, 8 to 11 amino acids, 9 to 11 amino acids, 9 to 13 amino acids or 9 to 14 amino acids. In particular, the peptide may have an amino acid length chosen from one of 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 amino acids, such as 9, 11, 13 or 15 amino acids.

The present invention incorporates peptide derivatives and peptide mimetics of any one of SEQ ID NO.s: 2, 4, 5, 8, 9, 11, 12, 20, 21, 26, 27-184 (optionally excluding one or more, or all, of SEQ ID NOs: 27, 28, 29, 30, 32, 34, 37, 40, 41).

Peptide derivatives include variants of a given SEQ ID NO and may include naturally occurring allelic variants and synthetic variants which have substantial amino acid sequence identity to the peptide sequence as identified in the wild type full length protein allergen.

Peptide derivatives may include those peptides having at least 60% amino acid sequence identity to one of SEQ ID NOs: 2, 4, 5, 8, 9, 11, 12, 20, 21, 26, 27-184 (optionally excluding one or more, or all, of SEQ ID NOs: 27, 28, 29, 30, 32, 34, 37, 40, 41) and which are capable of stimulating an immune response.

Typically a peptide derivative shows similar or improved MHC binding compared to the parent sequence, e.g. one of SEQ ID NOS: 2, 4, 5, 8, 9, 11, 12, 20, 21, 26, 27-184 (optionally excluding one or more, or all, of SEQ ID NOs: 27, 28, 29, 30, 32, 34, 37, 40, 41). Preferably a peptide derivative shows promiscuous binding to MHC Class II molecules.

Peptide derivatives may include peptides having at least one amino acid modification (e.g. addition, substitution, and/or deletion of one or more amino acids) compared to one of SEQ ID NOs: 2, 4, 5, 8, 9, 11, 12, 20, 21, 26, 27-184 (optionally excluding one or more, or all, of SEQ ID NOs: 27, 28, 29, 30, 32, 34, 37, 40, 41).

Peptide derivatives preferably differ from one of SEQ ID NOS: 2, 4, 5, 8, 9, 11, 12, 20, 21, 26, 27-184 (optionally excluding one or more, or all, of SEQ ID NOs: 27, 28, 29, 30, 32, 34, 37, 40, 41) by less than 5 amino acids. More preferably, the number of different amino acids is 4 amino acids or less, 3 amino acids or less, 2 amino acids or less or only 1 amino acid.

Peptide derivatives may arise through natural variations or polymorphisms which may exist between the members of a protein allergen family from which the peptide is derived. All such derivatives are included within the scope of the invention.

Peptide derivatives may result from natural or non-natural (e.g. synthetic) interventions leading to addition, replacement, deletion or modification of the amino acid sequence of one of SEQ ID NOs: 2, 4, 5, 8, 9, 11, 12, 20, 21, 26, 27-184 (optionally excluding one or more, or all, of SEQ ID NOs: 27, 28, 29, 30, 32, 34, 37, 40, 41).

Conservative replacements and modifications which may be found in such polymorphisms may be between amino acids within the following groups:
(i) alanine, serine, threonine;
(ii) glutamic acid and aspartic acid;
(iii) arginine and leucine;
(iv) asparagine and glutamine;
(v) isoleucine, leucine and valine;
(vi) phenylalanine, tyrosine and tryptophan;
(vii) methionine and leucine;
(viii) cysteine and valine.

Peptide derivatives may be peptide truncates of one or more of SEQ ID NOs: 2, 4, 5, 8, 9, 11, 12, 20, 21, 26, e.g. one or more of SEQ ID NOs: 27-184 (optionally excluding one or more, or all, of SEQ ID NOs: 27, 28, 29, 30, 32, 34, 37, 40, 41). A peptide truncate has the same amino acid sequence as one of SEQ ID NOs: 2, 4, 5, 8, 9, 11, 12, 20, 21, 26, 27-184 (optionally excluding one or more, or all, of SEQ ID NOs: 27, 28, 29, 30, 32, 34, 37, 40, 41) except for the deletion of one or more amino acids, 1, 2, 3, 4, or 5 amino acids may be deleted to provide a peptide truncate. A set of peptide truncates may be prepared in which 1, 2, 3, 4 or 5 amino acids are absent from either the C- or N-terminus of one of SEQ ID NOs: 2, 4, 5, 8, 9, 11, 12, 20, 21, 26, 27-184 (optionally excluding one or more, or all, of SEQ ID NOs: 27, 28, 29, 30, 32, 34, 37, 40, 41), e.g. one of SEQ ID NOs:27-184 to provide a set of up to 10 peptide truncates. Whilst peptide truncates may be prepared by removing the required number of amino acids from the C- or N-terminus it is preferred to directly synthesise the required shorter peptide in accordance with the amino acid sequence of the desired peptide truncate.

Peptide truncates can also be synthesised to have a sequence that corresponds to one of SEQ ID NOs: 2, 4, 5, 8, 9, 11, 12, 20, 21, 26, 27-184 (optionally excluding one or more, or all, of SEQ ID NOs: 27, 28, 29, 30, 32, 34, 37, 40, 41), e.g. one of SEQ ID NOs:27-184, where 1, 2, 3, 4 or 5 amino acids in internal positions in the peptide are deleted.

Peptide derivatives may also be provided by modifying one of SEQ ID NO.s: 2, 4, 5, 8, 9, 11, 12, 20, 21, 26, 27-184 (optionally excluding one or more, or all, of SEQ ID NOs: 27, 28, 29, 30, 32, 34, 37, 40, 41) to resist degradation of the peptide. FIG. 10 summarises modifications that may be made to the peptides to help resist peptide degradation and enhance peptide half-life in vitro and in vivo. These modifications may improve in vitro peptide stability and long-term storage. FIG. 10 also indicates enhancing sequences that may increase the rate of reaction of an adjacent or nearby amino acid.

Peptide derivatives may be provided by modifying one of SEQ ID NO.s: 2, 4, 5, 8, 9, 11, 12, 20, 21, 26, 27-184 (optionally excluding one or more, or all, of SEQ ID NOs: 27, 28, 29, 30, 32, 34, 37, 40, 41) for protease resistance, for example by inclusion of chemical blocks for exoproteases.

SEQ ID NOs: 120, 121, 21, 123, 125, 126, 128, and 129 are derivatives in that each peptide comprises an C/V substitution compared to the corresponding parent allergen sequence.

Peptide derivatives may also be provided by modifying one of SEQ ID NO.s: 2, 4, 5, 8, 9, 11, 12, 20, 21, 26, 27-184 (optionally excluding one or more, or all, of SEQ ID NOs: 27, 28, 29, 30, 32, 34, 37, 40, 41), to alter the immunomodulatory properties of the peptide. These derivatives are sometimes referred to as altered peptide ligands (APLs) (25). APLs typically produce an altered immune response compared to the unaltered (e.g. wild type) peptide. For example, an APL may induce increased or decreased T cell activation, altered cytokine profile in activated T cells, and/or altered MHC binding compared to the unaltered peptide. Preferably an APL displays promiscuous binding of MHC molecules as described herein.

Peptide derivatives may be assayed for their ability to induce an immune response, e.g. T cell proliferation and/or cytokine production in a T cell population, in order to identify a peptide pharmacophore representing the minimal or optimised peptide epitope capable of stimulating an immune response and that may be useful in therapy. The immune response induced by a peptide may be one or more of:
(i) in vitro T cell proliferation, e.g. as measured by peptide stimulation of bromodeoxyuridine or ³H-thymidine incorporation in in vitro cultured PBMC, and/or
(ii) secretion of cytokines, e.g. IFNγ and/or IL-4, by in vitro cultured PBMC or T cells, e.g. T helper cells, and/or
(iii) a Th1 or Th2 response (e.g. as measured by secretion of cytokines such as IFNγ or IL-4 respectively).

Peptide derivatives such as APLs may be screened for MHC binding, in particular for binding to HLA Class II molecules.

The invention includes a method of identifying a peptide derivative of one of SEQ ID NOs: 2, 4, 5, 8, 9, 11, 12, 20, 21, 26, 27-184 (optionally excluding one or more, or all, of SEQ ID NOs: 27, 28, 29, 30, 32, 34, 37, 40, 41) that is capable of stimulating an immune response. The method comprises the steps of (i) providing a candidate peptide derivative and (ii) testing the ability of the candidate peptide derivative to induce an immune response.

Part (i) may comprise synthesising the candidate peptide derivative, which may be a peptide mimetic or APL. Alternatively, part (i) may comprise chemically modifying the structure of one of SEQ ID NOs: 2, 4, 5, 8, 9, 11, 12, 20, 21, 26, 27-184 (optionally excluding one or more, or all, of SEQ ID NOs: 27, 28, 29, 30, 32, 34, 37, 40, 41) so as to produce a candidate peptide derivative. Part (i) may comprise synthesis of peptide truncates or derivatives. The candidate peptide derivative will preferably have at least 60% sequence identity to one of SEQ ID NOs: 2, 4, 5, 8, 9, 11, 12, 20, 21, 26, 27-184 (optionally excluding one or more, or all, of SEQ ID NOs: 27, 28, 29, 30, 32, 34, 37, 40, 41).

Chemical modification of one of SEQ ID NOs: 2, 4, 5, 8, 9, 11, 12, 20, 21, 26, 27-184 (optionally excluding one or more, or all, of SEQ ID NOs: 27, 28, 29, 30, 32, 34, 37, 40, 41) may, for example, comprise deletion of one or more amino acids, addition of one or more amino acids or chemical modification of one or more amino acid side chains.

Part (ii) may comprise screening a candidate peptide derivative for MHC binding, in particular for binding to HLA Class II molecules. Especially, part (ii) may comprise testing a candidate peptide derivative for promiscuous binding to MHC Class II molecules. In silico screening may be carried out using virtual HLA Class II matrices, such as the ProPred software described herein. An in vitro binding assay may be used to assess binding to HLA Class II molecules, such as the ProImmune Reveal® assay described herein.

Preferably a peptide derivative, e.g. an APL, is a promiscuous binder of MHC Class II alleles. Typically a promiscuous binding epitope binds over 50%, for example, at least 60% or at least 70%, of the HLA-DR alleles expressed by European Americans. The 11 most common alleles expressed by European Americans are shown in FIG. 11. Preferably a promiscuous binding epitope binds one of at least 2, 3, 4, 5, 6, 7, 8, 9, 10 or all 11 of the HLA-DR alleles in FIG. 11. In one aspect a peptide derivative may bind at least 2, 3, 4, 5, 6, 7, 8, 9, 10 or 11 of the alleles in FIG. 11 and also the HLA-DR allele *1401. The method may therefore comprise selecting a peptide that binds promiscuously.

Part (ii) may comprise contacting the candidate peptide derivative with a population of T cells and assaying T cell proliferation. Additionally, or alternatively, part (ii) may comprise contacting the candidate peptide derivative with a population of T cells and monitoring cytokine production, such as production of IFNγ and/or IL-4. The T cells are preferably T helper cells. The T cells may be provided as an in vitro culture of PBMC.

The method may further comprise the step of selecting one or more candidate peptide derivatives that stimulate T cell proliferation and detecting the production of cytokines in order to determine the induction of a Th1 or Th2 response. Preferably, the method comprises detection of IFNγ and/or IL-4. The method may further comprise selecting a peptide that induces a Th1 or Th2 response.

Methods according to the present invention may be performed in vitro or in vivo. The term “in vitro” is intended to encompass experiments with cells in culture whereas the term “in vivo” is intended to encompass experiments with intact multi-cellular organisms. Where the method is performed in vitro it may comprise a high throughput screening assay. Test compounds used in the method may be obtained from a synthetic combinatorial peptide library, or may be synthetic peptides or peptide mimetic molecules. Method steps (i) and (ii) are preferably performed in vitro, e.g. in cultured cells. Cells may be of any suitable cell type, e.g. mammalian, bacterial or fungal. Host cell(s) may be non-human, e.g. rabbit, guinea pig, rat, mouse or other rodent (including cells from any animal in the order Rodentia), cat, dog, pig, sheep, goat, cattle, horse, non-human primate or other non-human vertebrate organism; and/or non-human mammalian; and/or human. Suitable cells, e.g. PBMCs, may be obtained by taking a blood sample.

Part (ii) of the method may additionally comprise testing a candidate peptide derivative in animal models or patient populations for therapeutic effects on fungal allergy or fungal infection.

Peptides according to the present invention may be useful in the prevention or treatment of disease. In particular, peptides according to the present invention may be used to prepare pharmaceutical compositions. The pharmaceutical compositions may comprise medicaments or vaccines.

A pharmaceutical composition may be provided comprising a predetermined quantity of one or more peptides according to the present invention. Pharmaceutical compositions according to the present invention may be formulated for clinical use and may comprise a pharmaceutically acceptable carrier, diluent or adjuvant.

Pharmaceutical compositions of the invention are purified reproducible preparations which are suitable for human therapy. Preferred compositions of the invention comprise at least one isolated, purified peptide, free from all other polypeptides or contaminants, the peptide having a defined sequence of amino acid residues which comprises at least one T cell epitope of an antigen of interest.

As used herein, the term “isolated” refers to a peptide which is free of all other polypeptides, contaminants, starting reagents or other materials, and which is not conjugated to any other molecule.

A pharmaceutical composition of the invention is capable of down regulating an antigen specific immune response to an antigen of interest (e.g. Alt a 1 or Alt a 5) in a population of humans or animals subject to the antigen specific immune response such that disease symptoms are reduced or eliminated, and/or the onset or progression of disease symptoms is prevented or slowed.

Compositions and methods of the invention may be used to treat sensitivity to protein allergens in humans such as allergies to fungi, particularly to Alternaria spp.

Accordingly, in a further aspect of the invention a peptide combination or peptide according to the present invention is provided for use in the prevention or treatment of disease.

In another aspect of the present invention a peptide combination or peptide according to the present invention is provided for use in a method of medical treatment. The medical treatment may comprise treatment of a disease, e.g. allergic disease.

In another aspect of the present invention the use of a combination of peptides or a peptide according to the present invention in the manufacture of a medicament for the prevention or treatment of disease is provided.

In another aspect of the present invention a method is provided for preventing or treating disease in a patient in need of treatment, the method comprising administering to the patient a therapeutically effective amount of a combination of peptides or peptide or pharmaceutical composition according to the present invention.

In accordance with the present invention methods are also provided for the production of pharmaceutically useful compositions, which may be based on a peptide combination, peptide or peptide derivative according to the present invention. In addition to the steps of the methods described herein, such methods of production may further comprise one or more steps selected from:
(a) identifying and/or characterising the structure of a selected peptide combination, peptide or peptide derivative;
(b) obtaining the peptide combination, peptide or peptide derivative;
(c) mixing the selected peptides;
(d) mixing the selected peptide(s) or peptide derivative(s) with a pharmaceutically acceptable carrier, adjuvant or diluent.

For example, a further aspect of the present invention relates to a method of formulating or producing a pharmaceutical composition for use in the treatment of disease, the method comprising identifying a combination of peptides, peptide or peptide derivative(s) in accordance with one or more of the methods described herein, and further comprising one or more of the steps of:
(i) identifying the peptide combination, peptide(s) or peptide derivative(s); and/or
(ii) formulating a pharmaceutical composition by mixing the selected peptide(s) or peptide derivative(s), with a pharmaceutically acceptable carrier, adjuvant or diluent.

As such, the method may comprise providing a peptide or peptides which peptide(s) comprise(s) the sequence of one of SEQ ID NOs: 2, 4, 5, 8, 9, 11, 12, 20, 21, 26, 27-184, and formulating a pharmaceutical composition by mixing the selected peptide(s) or peptide derivative(s) with a pharmaceutically acceptable carrier, adjuvant or diluent.

The peptide(s) or peptide derivative(s) may be present in the pharmaceutical composition in the form of a physiologically acceptable salt.

In some embodiments methods of medical treatment involve administering more than one peptide according to the invention to the patient. Administering two, three or more peptides derived from a single allergen may be used to ensure that peptide epitopes that bind to a large number of HLA alleles are provided. For example, one may wish to ensure that the treatment includes administration of peptide epitopes derived from a given allergen that collectively bind to all 11 alleles of FIG. 11. Administration of multiple peptides may be simultaneous, separate or sequential and may form part of a combination therapy.

Accordingly, a pharmaceutical composition or medicament according to the invention may comprise more than one peptide of the invention. Such compositions and medicaments may contain more than one peptide and/or peptide derivative and/or peptide mimetic according to the invention, for example, 1, 2, 3, 4, 5, 6, 7, 8, 9, 10, 11, 12, 13, 14, 15, 16, 17, 18, 19, 20 or more peptides, peptide derivatives and/or peptide mimetics.

In yet a further aspect of the present invention nucleic acids encoding peptides according to the present invention are provided, together with their complementary sequences. The nucleic acid may have a maximum length of 1000 nucleotides, more preferably one of 200, 190, 180, 170, 160, 150, 140, 130, 120, 110, 100, 90, 80, 70, 60, 50, 40, 30, 25 nucleotides. The nucleic acid may have a minimum length of 24 nucleotides, more preferably one of 27, 30, 35, 40, 45, 50, 55 or 60 nucleotides.

A nucleic acid vector having nucleic acid encoding a peptide of the present invention is also provided. The vector may be an expression vector, e.g. a plasmid, in which a nucleic acid sequence encoding a peptide of the present invention is operably linked to a suitable promoter and/or other regulatory sequence. A host cell transfected with such a vector is also provided.

In this specification the term “operably linked” may include the situation where a selected nucleotide sequence and regulatory or control nucleotide sequence are covalently linked in such a way as to place the expression of a nucleotide sequence under the influence or control of the regulatory sequence. Thus a regulatory or control sequence is operably linked to a selected nucleotide sequence if the regulatory sequence is capable of effecting transcription of a nucleotide sequence which forms part or all of the selected nucleotide sequence. Where appropriate, the resulting transcript may then be translated into a desired peptide.

The vector may be configured to enable transcription of mRNA encoding the peptide upon transfection into a suitable cell. Transcribed mRNA may then be translated by the cell such that the cell expresses the peptide.

A cell having a nucleic acid sequence encoding a peptide of the present invention operably linked to a suitable promoter and/or other transcription regulatory element or control sequence integrated in the genome of the cell is also provided.

Nucleic acids according to the invention may be single or double stranded and may be DNA or RNA.

Diseases or conditions that may be prevented or treated include allergic disease. Examples of allergic disease include asthma, allergic asthma, fungal asthma, SAFS, ABPA, allergic bronchopulmonary mycoses, allergic sinusitis, rhinitis, allergic rhinitis, hypersensitivity pneumonitis, atopic eczema. Other diseases or conditions that may be prevented or treated include fungal infection, Aspergillosis (e.g. invasive, non-invasive, chronic pulmonary, aspergilloma).

Peptide therapy may comprise the use of peptides according to the invention in the prevention/prophylaxis of disease or in the treatment of disease. As such, therapy may comprise relief or reduction of symptoms such as airway inflammation, difficulty in breathing, swelling, itchiness, allergic rhinitis, allergic sinusitis, eosinophilia, hypersensitivity to fungal allergens and/or spores. A reduction in asthmatic symptoms may be measured by conventional techniques, such as measuring peak flow, white blood cell count, patch testing.

Peptides according to the present invention may be useful as prophylactics for the prevention of allergy responses to fungal allergens, particularly allergens from Alternaria alternata such as Alt a 1 or Alt a 5.

Patients to be treated may be any animal or human. The patient may be a non-human mammal, but is more preferably a human. Subjects, individuals or patients to be treated may be male or female. In one aspect, patients are of a selected ethnicity, which may include one or more of (by birth or residence): (i) European, (ii) from a Member State of the European Union, (iii) North American, e.g. from USA and/or Canada. Patients to be treated may be European American and/or Caucasian.

Medicaments and pharmaceutical compositions according to aspects of the present invention may be formulated for administration by a number of routes, including intravenous, intradermal, intramuscular, oral and nasal. The medicaments and compositions may be formulated in fluid or solid form. Fluid formulations may be formulated for administration by injection to a selected region of the human or animal body. Pharmaceutical compositions may comprise peptides encapsulated in liposomes, e.g. formed from polyglycerol esters.

Administration of peptides or pharmaceutical compositions for therapeutic purposes is preferably in a “therapeutically effective amount”, this being sufficient to show benefit to the individual. The actual amount administered, and rate and time-course of administration, will depend on the nature and severity of the disease being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

A composition may be administered alone or in combination with other treatments, either simultaneously or sequentially, dependent upon the condition to be treated.

Efficacious peptide immunotherapy may require the repeat administration of a pharmaceutical composition according to the present invention. For example, a dosage regime comprising a series of injections of the pharmaceutical composition may be required in order to treat existing allergic disease symptoms and to provide a vaccination effect against future allergic disease caused by fungal allergens.

Peptides comprising or consisting of SEQ ID NOS: 2, 4, 5, 8, 9, 11, 12, 20, 21, 26, 27-184 (optionally excluding one or more, or all, of SEQ ID NOs: 27, 28, 29, 30, 32, 34, 37, 40, 41) are disclosed along with variants and derivatives thereof, including peptides having conservative alterations. These peptides are each proposed for use in the treatment of fungal allergy, preferably allergic disease caused by A. alternata.

The peptides identified may be synthesised by standard techniques (e.g. using commercially available peptide synthesis services such as that provided by Invitrogen, Carlsbad, Calif., USA) and tested for use as a therapeutic or vaccine against fungal infection or fungal allergy.

Various methods of chemically synthesizing peptides are known in the art such as solid phase synthesis which has been fully or semi-automated on commercially available peptide synthesizers. Synthetically produced peptides may then be purified to homogeneity (i.e. at least 90%, more preferably at least 95% and even more preferably at least 97% purity), free from all other polypeptides and contaminants.

Peptide compositions may then be characterized by a variety of techniques well known to those of ordinary skill in the art such as mass spectroscopy, amino acid analysis and sequencing and HPLC.

Peptides useful in the methods of the present invention may also be produced using recombinant DNA techniques in a host cell transformed with a nucleic acid sequence coding for such peptide. When produced by recombinant techniques, host cells transformed with nucleic acid encoding the desired peptide are cultured in a medium suitable for the cells and isolated peptides can be purified from cell culture medium, host cells, or both using techniques known in the art for purifying peptides and proteins including ion-exchange chromatography, ultra filtration, electrophoresis or immunopurification with antibodies specific for the desired peptide. Peptides produced recombinantly may be isolated and purified to homogeneity, free of cellular material, other polypeptides or culture medium for use in accordance with the methods described above.

Pharmaceutical compositions of the invention should be sterile, stable under conditions of manufacture, storage, distribution and use and should be preserved against the contaminating action of microorganisms such as bacteria and fungi. A preferred means for manufacturing a pharmaceutical composition of the invention in order to maintain the integrity of the composition is to prepare the formulation of peptide and pharmaceutically acceptable carrier(s) such that the composition may be in the form of a lyophilized powder which is reconstituted in a pharmaceutically acceptable carrier, such as sterile water, just prior to use.

Biodegradable poly(D,L-lactic-co-glycolic) acid (PGLA) particles has been suggested for delivery of peptides for treatment of allergy (Scholl et al. Immunol. Allergy Clin. N. Am. 2006. 26:349-364.).

T-cell epitope validation can be performed by assaying peptide-induced proliferation of peripheral blood mononuclear cells (PBMC) obtained from subjects having fungal allergy or fungal infection and from control subjects not having fungal allergy or fungal infection. HLA-DR typing of subject PBMCs may also be performed to confirm the promiscuous binding nature of the peptides.

The status of the proliferated T helper cells may also be determined and used to assist in validation of peptides as therapeutic or vaccine candidates. Th1 cells participate in cell-mediated immunological responses. Th2 cells participate in antibody mediated immunity.

Th1/Th2 status can be determined by examining the cytokine profile of the proliferated cells (27). Production of interferon γ (IFNγ) and optionally one or more of interleukin 2 (IL-2), tumor necrosis factor β (TNFβ) and granulocyte-macrophage colony stimulating factor (GM-CSF) is indicative of Th1 status. Typically this indicates a non-allergic cellular immune response. Production of interleukin 4 (IL-4) and optionally one or more of interleukin 3 (IL-3), interleukin 5 (IL-5), interleukin 6 (IL-6), interleukin 10 (IL-10) and interleukin 13 (IL-13) is indicative of Th2 status. Often this is associated with an allergic Th2 response. Production of both IFNγ and IL-4 is indicative of Th0 status. Production of IL-10 is associated with a Treg non-allergic response. (27)

Th2 cells play an important role in the immunological processes of allergic asthma (11) and Th2 associated cytokines such as IL-4, IL-5, IL-9 and IL-13 are implicated in the development of allergen specific Th2 cells, IgE production, airway eosinophilia and airway hyper-responsiveness. Inhibition or suppression of allergen-specific Th2 cells and their cytokines provides a strategy for intervention.

Such inhibition or suppression may be achieved by selecting Th1 stimulating peptides leading to suppression of the Th2 response (11). Alternatively, Th2 stimulating peptides administered via different routes (oral, lymph node injection or intravenous) and by specific dose variation may be used to suppress an allergen induced Th2 response through a bystander effect. The bystander effect is defined as an influence on the immune response to a particular antigen(s) of interest by the immune response to other unrelated antigens, usually mediated by a local cytokine and cellular environment. The bystander effect can result in an amplification of an immune response (22) or a suppression of a response (23).

Low-dose T-cell epitope peptides from allergen proteins are proposed to cause antigen specific hypo-responsiveness associated with the induction of a suppressive population of CD4+ T cells, together with up regulation of surface CD5 levels on antigen-specific T cells (12). Intravenous injection of a single peptide induces a bystander suppression and thus can provide protection against a multicomponent allergen trigger (13).

Accordingly, in addition to assaying for T cell proliferation (e.g. based on Bromodeoxyuridine (BRdU) or ³H thymidine incorporation), cytokine assays' may be performed to detect secretion of one or more of IFNγ, IL-2, TNFβ, GM-CSF, IL-4, IL-3, IL-5, IL-6, IL-10 and IL-13. Further assays to detect the presence of an IgE response and/or eosinophilia may also be performed.

Human T cell stimulating activity can be tested by culturing T cells obtained from an individual sensitive to a predetermined protein antigen with a peptide derived from the antigen and determining whether proliferation of T cells occurs in response to the peptide as measured, e.g., by cellular uptake of ³H thymidine. Stimulation indices for responses by T cells to peptides can be calculated as the maximum counts per minute (CPM) in response to a peptide divided by the control CPM. A T cell stimulation index (S.I.) equal to or greater than two times the background level is considered “positive”. Positive results are used to calculate the mean stimulation index for each peptide for the group of peptides tested.

Preferred peptides have a mean T cell stimulation index of greater than or equal to 2.0. A peptide having a T cell stimulation index of greater than or equal to 2.0 is considered useful as a therapeutic agent. Preferred peptides have a mean T cell stimulation index of at least 2.5, more preferably at least 3.5, even more preferably at least 4.0, and most preferably at least 5.0.

The positivity index (P.I.) for a peptide is determined by multiplying the mean T cell stimulation index by the percent of individuals, in a population of individuals tested, sensitive to the antigen being tested (e.g., preferably at least 9 individuals, more preferably at least 16 individuals or more, more preferably at least 20 individuals or more, or even more preferably at least 30 individuals or more), who have T cells that respond to the peptide. The positivity index represents the strength of a T cell response to a peptide (S.I.) and the frequency of a T cell response to a peptide in a population of individuals sensitive to the antigen being tested. Preferred peptides may also have a positivity index (P.I.) of at least about 100, more preferably at least 150, even more preferably at least about 200 and most preferably at least about 250.

Cytokine production may be analysed using any of the methods described herein. One such method employs an Enzyme-linked ImmunoSpot (ELISPOT) assay. The ELISPOT assay will allow the analysis of cells at the single cell level for cytokine production, and thus provides a method for determining the number of individual T cells secreting a cytokine after stimulation with a specific antigen or peptide (28). The ELISPOT assay typically uses two high-affinity cytokine-specific antibodies directed against different epitopes on the same cytokine molecule. Spots are generated with a colorimetric reaction in which soluble substrate is cleaved, leaving an insoluble precipitate at the site of the reaction. The spot represents a foot-print of the original cytokine producing cell.

The number of spots is a direct measurement of the frequency of cytokine-producing T cells.

The production of cytokines by T-cells in PMBC cell cultures in response to allergen indicates that stimulation has occurred and identification of the cytokine pattern allows a comparison of the type of cellular response.

Peptides selected through in vitro validation assays such as those described above may be tested in animal models or patient populations for therapeutic effects on fungal allergy or fungal infection, e.g. as described in Kheradmand et al (24). For example, a mouse model may be used, such as BALB/c(H2^{d}) mice. Patients or animals may receive a series of peptide formulations, e.g. by injection, and fungal infection or allergy symptoms and characteristics monitored. Such symptoms and characteristics may include airway inflammation, eosinophilia, rhinitis, cytokine secretion, Th1 or Th2 response status. Suitably, a control patient population receiving placebo formulations may be used to assess efficacy of the peptide formulation.

Simultaneous, Sequential or Separate Administration

In some aspects and embodiments of the present invention two or more peptides may be administered separately, either simultaneously or sequentially, or in a combined preparation.

Simultaneous administration refers to administration of the two or more peptides together, for example as a pharmaceutical composition containing both peptides, or immediately after each other and optionally via the same route of administration.

Sequential administration refers to administration of one of the peptides followed after a given time interval by separate administration of another (preferably different) peptide. It is not required that the two peptides are administered by the same route, although this is the case in some embodiments. The time interval may be any time interval.

Simultaneous or sequential administration is intended such that both peptides are delivered to the patient so that their independent actions on the patient may be exhibited in the same or an overlapping time frame. In some embodiments of sequential administration the time interval is selected such that the peptides are expected to be administered to the patient so as to allow for a combined, additive or synergistic effect of the two or more peptides.

Administration of peptides may be at substantially the same time, and may involve administration of a single pharmaceutical composition or medicament containing the two or more peptides. Where the peptides are given in separate pharmaceutical compositions the time interval between administrations may be any one of 5 minutes or less, 10 minutes or less, 15 minutes or less, 20 minutes or less, 25 minutes or less, 30 minutes or less, 45 minutes or less, 60 minutes or less, 90 minutes or less, 120 minutes or less, 180 minutes or less, 240 minutes or less, 300 minutes or less, 360 minutes or less, or 720 minutes or less, or 1 day or less, or 2 days or less.

Peptide Mimetics

The designing of mimetics to a known pharmaceutically active compound is a known approach to the development of pharmaceuticals based on a “lead” compound. This might be desirable where the active compound is difficult or expensive to synthesise or where it is unsuitable for a particular method of administration, e.g. some peptides may be unsuitable active agents for oral compositions as they tend to be quickly degraded by proteases in the alimentary canal. Mimetic design, synthesis and testing is generally used to avoid randomly screening large numbers of molecules for a target property.

There are several steps commonly taken in the design of a mimetic from a compound having a given target property. Firstly, the particular parts of the compound that are critical and/or important in determining the target property are determined. In the case of a peptide, this can be done by systematically varying the amino acid residues in the peptide, e.g. by substituting each residue in turn. These parts or residues constituting the active region of the compound are known as its “pharmacophore”.

Once the pharmacophore has been found, its structure is modelled according to its physical properties, e.g. stereochemistry, bonding, size and/or charge, using data from a range of sources, e.g. spectroscopic techniques, X-ray diffraction data and NMR. Computational analysis, similarity mapping (which models the charge and/or volume of a pharmacophore, rather than the bonding between atoms) and other techniques can be used in this modelling process.

In a variant of this approach, the three-dimensional structure of the ligand and its binding partner are modelled. This can be especially useful where the ligand and/or binding partner change conformation on binding, allowing the model to take account of this in the design of the mimetic.

A template molecule is then selected onto which chemical groups which mimic the pharmacophore can be grafted. The template molecule and the chemical groups grafted on to it can conveniently be selected so that the mimetic is easy to synthesise, is likely to be pharmacologically acceptable, and does not degrade in vivo, while retaining the biological activity of the lead compound. The mimetic or mimetics found by this approach can then be screened to see whether they have the target property, or to what extent they exhibit it. Further optimisation or modification can then be carried out to arrive at one or more final mimetics for in vivo or clinical testing.

With regard to the present invention, a peptide mimetic is one form of peptide derivative. A method of identifying a peptide derivative capable of stimulating an immune response may comprise the step of modifying the peptide structure to produce a peptide mimetic. This peptide mimetic may optionally be subject to testing in a T cell proliferation assay, and/or in cytokine secretion assays (e.g. assaying for IFN γ or IL-4 production). This process of modification of the peptide or peptide mimetic and testing may be repeated a number of times, as desired, until a peptide having the desired effect, or level of effect, on T cell proliferation and/or cytokine secretion is identified.

The modification steps employed may comprise truncating the peptide or peptide mimetic length (this may involve synthesising a peptide or peptide mimetic of shorter length), substitution of one or more amino acid residues or chemical groups, and/or chemically modifying the peptide or peptide mimetic to increase stability, resistance to degradation, transport across cell membranes and/or resistance to clearance from the body.

Altered Peptide Ligands (APLs)

Altered peptide ligands (APLs) are modified versions of peptide epitopes, with altered immunomodulatory properties (25).

A Th1-skewing APL has been reported, having a single 336N/A substitution compared to the wild type peptide epitope (implicated in allergic asthma) and which inhibits the allergic Th2 response in a mouse model of allergic asthma (11).

An APL of an immunodominant epitope of lipocalin allergen Bos d2 has also been reported which produces a Th1/Th0 response in vitro compared to the Th2/Th0 response induced by the wild type epitope (29). The T cell population induced by the APL are cross-reactive with the wild type epitope (29).

Changes in the residues flanking the core epitope of the immunodominant myelin basic protein (MBP) peptide 84-102 have been reported to alter both MHC binding and T cell activation, the latter independently of MHC binding (30). It is suggested that C-terminal basic residues may enhance processing and presentation of an epitope.

With regard to the present invention, an APL is one form of peptide derivative.

An APL typically induces an altered immune response compared to the unaltered (usually wild type) peptide.

Immunomodulatory properties that may be altered include one or more of:

T Cell Activation

T cell activation in response to the APL may be increased or decreased compared to the unmodified peptide. Activation may occur at a higher or lower dose of peptide. Some APLs are unable to originate T cell signalling and lead to an impairment of T cell activation (antagonist APLs). Some APLs elicit some but not all of the signals for full T cell activation (partial agonist APLs) (25).

Cytokine Profile

T cells activated by the peptide may secrete a different pattern of cytokines than T cells activated by the unmodified peptide. Thus, a modified peptide may induce a different type of T cell response, e.g. Th1 in place of Th2, Treg in place of Th2, or Th1 in place of Treg.

MHC Binding

An APL may show altered MHC binding compared to the unmodified peptide. In the present case it is preferred that an APL shows similar or improved MHC binding compared to the unaltered peptide. In particular it is preferred that an APL is a promiscuous binder of MHC Class II alleles.

The T cells activated by the APL may be cross reactive with the unmodified or wild type epitope.

A method of identifying a peptide derivative capable of stimulating an immune response as described herein may comprise the step of modifying the peptide structure to produce an APL with altered immunomodulatory properties as described herein.

Modifying the peptide may comprise modifying, substituting, adding or deleting one or more amino acids. Modifications which may be found in peptide derivatives are described herein.

For example, modifying a peptide may comprise systematically altering one or more amino acids in the peptide, e.g. substituting each amino acid in turn. For example, an initial screen may use an alanine scan to prepare a set of peptide derivatives from a starting peptide, each derivative being substituted with an alanine at a single position (Janssen et al. J. Immunol. 2000. 164:580-588.).

Modifying a peptide may comprise adding 1, 2, or 3 (or more) amino acids at the N-terminal end, the C-terminal end, or at both N-terminal and the C-terminal end.

Modification may be at an amino acid within any of SEQ ID NOS:1-184. Alternatively, modification may be at an amino acid in a region flanking any of these sequences, such as the N-terminal and/or C-terminal 1, 2, 3, 4, 5 or 6 amino acids. For example, one or more additional amino acids may be added, substituted or chemically modified at the N-terminal and/or C-terminal region of an epitope. Preferably one or more basic amino acids is included at the C-terminal end of a peptide.

Binding core 9-mers of class II DR epitopes have a general pattern of amino acid side chains important in binding to the MHC and important for binding of the MHC/peptide complex to the T-cell receptor. For a typical peptide epitope, alterations of residues P1, P4, P6 or P9 can alter peptide binding strength to MHC alleles while alterations of P2, P3, P5, P7 and P8 can alter the interactions of MHC/peptide complex with T-cell receptors. Altering the strength of binding of the MHC/peptide complex to the T-cell receptor is known to have the ability to change the fate of the original T-cell receptor clone as to cytokine polarization and/or interact with structurally related T-cell receptor clones not induced by the original peptide.

Candidate APL(s) may be assessed for binding to MHC Class II molecules, in particular HLA Class II molecules such as HLA-DR alleles. Typically an APL is tested for binding to HLA DR alleles which occur at a frequency of at least 40% in the European-American population, for example at least 50%, 60%, 70%, 80% or 90% in the population. Preferably an APL is tested for binding to at least 2, 3, 4, 5, 6, 7, 8, 9, 10, or 11 of the alleles in FIG. 11 (and optionally also to the HLA DR *1401 allele).

Preferably an APL exhibits substantially similar or improved binding compared to the unaltered peptide. Preferably an APL shows promiscuous binding to HLA Class II molecules as described herein.

MHC binding may be assessed using in silico screening. Typically in silico screening, such as the ProPred software described herein, comprises use of virtual HLA Class II matrices. Additionally or alternatively, MHC binding may be assessed using an in vitro binding assay, such as the ProImmune REVEAL® assay described herein.

Candidate APL(s) may be subject to testing in a T cell proliferation assay, and/or in cytokine secretion assays (e.g. assaying for IFN γ or IL-4 production) to determine the nature of the T cell response to the APL. For example, epitope specific T-cell lines and clones can be isolated from sensitized allergic donors. An APL modified from the native sequence may cross-react with the original clones induced by the native peptide and/or it may induce new T-cell receptor clones. Using an original line or clone induced by the native epitope for testing with APLs allows precise characterization of proliferation/cytokine pattern changes on the original population of clones due to amino acid changes in the peptide. Specific APLs that exhibit the desired properties can be tested for effects on whole TCR populations from the targeted patient population.

APLs selected through in vitro validation assays such as those described above may be tested in animal models or patient populations for therapeutic effects on fungal allergy or fungal infection as described herein.

This process of modification of the peptide and testing may be repeated a number of times, as desired, until a peptide having the desired effect, or level of effect, on T cell proliferation and/or cytokine secretion is identified.

In one aspect a peptide derivative herein refers to an APL of any one of SEQ ID NOs: 2, 4, 5, 8, 9, 11, 12, 20, 21, 26, 27-184 (optionally excluding one or more, or all, of SEQ ID NOs: 27, 28, 29, 30, 32, 34, 37, 40, 41).

Peptide Solubility

For some applications it is desirable for the peptide to be soluble in a liquid, e.g. water, saline solution or another pharmaceutically acceptable liquid carrier. Some hydrophobic peptides may first be dissolved in DMSO or other solvents and diluted into aqueous solution. Where the hydrophobic character of the peptide prevents such an approach the peptide may be modified to improve solubility. Modification of the peptide may be achieved in several ways well known to one of skill in the art, including the following.

One type of modification involves alteration of the peptide amino acid sequence to provide a peptide derivative in which one or more hydrophobic amino acids are substituted with amino acids of moderate or low hydrophobicity or with charged or uncharged polar amino acids.

Another type of modification involves modification of the N- and/or C-terminal ends of the peptide. Peptide derivatives may be provided in which the N-terminus is free and charged (NH₂—) or blocked with an acetyl group (AC—) or with Biotin. The C-terminus may also be free and charged (—COOH) or blocked (—CONH₂).

Another type of modification involves addition of one, two or three amino acids to the N- and/or C-terminus of the peptide to provide a longer peptide derivative. The additional amino acids may be any amino acids. In preferred embodiments the additional amino acids are chosen from the amino acids adjacent the N- or C-terminus of the peptide sequence as found in the protein amino acid sequence from which the peptide is derived. However, these may be modified to increase solubility.

Following modification to provide a peptide derivative the peptide derivative would be tested for retention of biological activity and for improvement in solubility.

Sequence Identity

Aspects of the invention concern compounds which are isolated peptides/polypeptides comprising an amino acid sequence having a sequence identity of at least 60% with a given sequence. Alternatively, this identity may be any one of 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity.

Percentage (%) sequence identity is defined as the percentage of amino acid residues in a candidate sequence that are identical with residues in the given listed sequence (referred to by the SEQ ID NO.) after aligning the sequences and introducing gaps if necessary, to achieve the maximum sequence identity, and not considering any conservative substitutions as part of the sequence identity. Sequence identity is preferably calculated over the entire length of the respective sequences.

Alignment for purposes of determining percent amino acid sequence identity can be achieved in various ways known to a person of skill in the art, for instance, using publicly available computer software such as ClustalW 1.82. T-coffee or Megalign (DNASTAR) software. When using such software, the default parameters, e.g. for gap penalty and extension penalty, are preferably used. The default parameters of ClustalW 1.82 are: Protein Gap Open Penalty=10.0, Protein Gap Extension Penalty=0.2, Protein matrix=Gonnet, Protein/DNA ENDGAP=−1, Protein/DNA GAPDIST=4.

Identity of nucleic acid sequences may be determined in a similar manner involving aligning the sequences and introducing gaps if necessary, to achieve the maximum sequence identity, and calculating sequence identity over the entire length of the respective sequences.

The invention includes the combination of the aspects and preferred features described except where such a combination is clearly impermissible or expressly avoided.

The section headings used herein are for organizational purposes only and are not to be construed as limiting the subject matter described.

Aspects and embodiments of the present invention will now be illustrated, by way of example, with reference to the accompanying figures. Further aspects and embodiments will be apparent to those skilled in the art. All documents mentioned in this text are incorporated herein by reference.

### BRIEF DESCRIPTION OF THE FIGURES

Embodiments and experiments illustrating the principles of the invention will now be discussed with reference to the accompanying figures in which:

FIG. 1. Cumulative distribution plot of background subtracted spot counts in % frequency. A, Control subjects (Control 1) and Alternaria allergic patients (Patient) total ELISPOT count distributions. B, Control 1 distribution with outliers removed (Control 2) and the normalized distribution of control counts with outliers removed (Normal Control 2).

FIG. 2. Charts showing response of control and patient populations to Alt a 1 peptides and corresponding Alt a 1 peptide hydrophilicity. A, Percent population response of control subject and Alternaria allergic patient populations with >9 IL-4 ELISPOT counts for each Alt a 1 peptide. B, Theoretical hydrophilicity of each Alt a 1 peptide.

FIG. 3. Table I: Patient Characteristics.

FIG. 4. TABLE II: Alt a 1 peptide-HLA binding prediction and in vitro HLA binding assay with DRB1*0101, 0301, 0401, 0701, 1101, 1301,1501.

FIG. 5. TABLE III: Alt a 1 peptide-HLA binding prediction to DRB1*0404, 0801, 1104, 1302.

FIG. 6. TABLE IV. ELISPOT counts of control subjects and Alternaria allergic patients exposed to Alt a 1 peptides.

FIG. 7. TABLE V. HLA typing of Alternaria allergic patients and controls.

FIG. 8. Table showing Alt a 1 peptide 15mer sequences.

FIG. 9. Table showing Alt a 5 peptide 15mer sequence.

FIG. 10. Table of conservative amino acid modifications indicating amino acid modifications that may be made to peptides of the invention in order to increase peptide resistance to degradation.

FIG. 11. Table of top 11 DRB1 alleles used in ProPred search. Alleles are shown by percentage population frequency present in European Americans.

FIG. 12. Amino acid sequence of Alt a 1 (UniProt Accession No. P79085).

FIG. 13. Amino acid sequence of Alt a 5 (UniProt Accession No. P42037).

FIG. 14. Table showing results of ELISPOT counts of Alternaria allergic patients exposed to Alt a 5 peptide SEQ ID NO:26.

### EXAMPLES

### Example 1

### Characterization and Selection of T-Cell Epitopes of the Major Alternaria alternata Allergen Alt a 1 for Peptide Immunotherapy

Methods

Subjects

Twenty three Alternaria allergic patients and 17 controls were recruited from the University of Barcelona allergy clinic (Barcelona, Spain). Alternaria patients were skin-prick test (SPT) positive to Alternaria extract (Diater). Controls were SPT negative to Alternaria and other fungi. Nasal provocation testing (NPT) with Alternaria extract was used to diagnose Alternaria specific allergic rhinitis as measured by acoustic rhinometry. The challenge was considered positive if the nasal challenge with the diluent was negative and the volume between the 2^{nd} and 5^{th} cm sections of the nose decreased >25% after the Alternaria challenge (20). All of the Alternaria allergic patients were NPT positive while all the control subjects were NPT negative. Subject histories were obtained and the results of further SPT are summarized in Table I. All of the Alternaria allergic patients were also positive for other aeroallergens including dust mite, pollen, epithelium derived from cat and/or dog, and/or other fungi such as Cladosporium herbarum, Aspergillus fumigatus, and/or Penicillium species. The presence of IgE to the A. alternata major allergen Alt a 1 was determined using ImmunoCAP and/or ImmunoCAP ISAC (Phadia) assay.

Epitope Prediction and Peptide Synthesis

The computational servers harboring ProPred and NetMHCIIpan 2.1 software packages were used to predict Alt a 1 peptides that promiscuously bind to multiple DRB1 alleles (21, 22). ProPred binding predictions were at a stringency threshold level 3 (default) and level 10 while NetMHCIIpan binding predictions used the default parameters for weak and strong binders. Predictions were obtained to the 11 most frequent DRB1 alleles found in the North American population of European descent (23): DRB1*0101, *0301, *0401, *0404, *0701, *0801, *1101, *1104, *1301, *1302, and *1501. NetMHCIIpan 2.2 was used for prediction of Alt a 1 peptides binding to DQB1 alleles using default parameters for weak and strong binders (24, 25). The 15mer peptides were subsequently synthesized by NEO-Peptide (Cambridge, Mass.) as an acetate salt with free N and C termini at a purity of >95% and were used to test validation of the prediction models. For synthesized peptides, cysteine residues were substituted with valine residues and/or methionine residues were substituted with leucine residues.

HLA Typing

DNA was isolated from 17 patients and 15 controls from whole blood or PBMCs with a Gene Elute Blood Genomic kit (Sigma). HLA typing of DRB1 to a four digit resolution was performed by the Histocompatibility and Immunogenetics Laboratory at Manchester Royal Infirmary (Manchester, United Kingdom).

MHC-Peptide Binding Assay

The MHC restriction of peptides from ProPred prediction were evaluated using the REVEAL Class II binding assay and Quick Check Stability Assay performed by ProImmune (Oxford, United Kingdom). In the REVEAL cell free in vitro assay, the binding of a peptide to an HLA molecule is determined by its ability to stabilize a MHC class II-peptide complex. Each MHC class II-peptide binding was scored relative to a validated proprietary T cell epitope control peptide. The score was determined as the percentage of the signal generated by the test peptide versus the level for the positive control peptide and reflects the on-rate properties of peptide. The off-rate properties of the peptide were determined by the Quick Check Stability Assay which measured the amount of peptide bound at time zero and time 24 hours at 37° C. The two signals were used to estimate a half-life which was multiplied by the REVEAL score and divided by 100 to yield the combined stability index. A stability index ≧1.0 was considered positive for MHC binding.

PBMC Collection and Preparation

Peripheral blood was obtained by venipuncture from Alternaria allergic patients and non-sensitized controls. Peripheral blood mononuclear cells (PBMCs) were isolated from heparinized blood by standard Ficoll density gradient centrifugation. PBMCs were washed with CTL (Cellular Technology Limited, Cleveland, Ohio, USA) wash medium containing RPMI-1640 with L-glutamine (Lonza, Basel, Switzerland) before being counted by haemocytometer using trypan blue stain (Sigma). PBMCs (10 million/ml) were resuspended in CTL-Cryo ABC serum-free freezing medium according to the manufacturer's protocol before being frozen overnight at −80° C. and transferred to and stored in liquid nitrogen until time of use.

IL-4 ELISPOT Analysis

Enzyme-linked immunospot assay (ELISPOT) analysis was performed utilizing 16 Alternaria patients and 11 controls. For an individual peptide the number of Alternaria patients tested ranged between 10-15 and the number of control subjects ranged between 7-9. Ten Alternaria patients were fully tested with all 22 peptides. For ELISPOT, the BD ELISPOT Human IL-4 Set (BD Biosciences, San Diego, Calif., USA) was used to analyze IL-4 production by human PBMCs. Plates were coated overnight at 4° C. with IL-4 capture antibody (BD Biosciences) and then washed 3 times with Dulbecco's Phosphate Buffer Saline (DPBS, Sigma). Plates were blocked with 1% BSA (Sigma) in PBS for 2 h at room temperature and washed 3 times with DPBS. Cryopreserved Alternaria patient and control subject PBMCs were thawed rapidly, washed, counted, and resuspended in CTL Test Medium supplemented with 2 mM L-glutamine (Sigma) and used at a concentration of 300,000 cells/well contained in 100 μl. Peptides were dissolved in DMSO to 50 mg/ml then diluted to 2 mg/ml with sterile H₂O and stored at −20° C. Prior to use, thawed peptides were diluted 1/100 in CTL Test medium supplemented with 2 mM L-glutamine and 100 μl was added to appropriate patient and control wells for a final peptide concentration of 10 μg/ml and a final DMSO concentration of 2.86 mM. 100 μl of CTL Test medium supplemented with 2 mM L-glutamine and 2.86 mM DMSO was added to patient and control subject no-peptide cell background control wells. Positive control wells contained 200,000 cells/well plus 5 μg/ml phytohemagglutinin (PHA) (Sigma) in a total of 200 μl. After incubation at 37° C., 5% CO₂ for 48 h, the cells were removed by washing 3 times with PBS and 4 times with PBS containing 0.05% Tween-20 (PBST). Biotinylated detection antibody was added and plates were kept at 4° C. overnight. After the wells were washed 3 times with PBST, Streptavidin-Horse Radish Peroxidase (HRP) conjugate provided in the BD ELISPOT Human IL-4 kit was added. After 1 hour at room temperature in the dark, wells were washed 2 times each with PBST and PBS alone and developed for 20-40 min with 3-amino-9-ethylcarbazole (AEC, BD Biosciences). The reaction was stopped by washing the wells with deionized water. The plates were dried and analyzed on the ImmunoSpot UV Core ELISPOT Plate Reader (Cellular Technology Limited).

Statistical Analysis

The two-independent sample Wilcoxon rank sum test was used for statistical analysis of ELISPOT data (26). A one-sided control<patient p-value was determined except for peptides 7-21 and 143-157 where a one-sided patient >control p-value was calculated. Outlier identification using g=1.5 and g=2.2 was performed as described (27). For correlation analysis the Pearson product moment correlation was used with a two-sided p-value.

Peptide Solubility

The theoretical average peptide hydrophilicity was calculated using the Hopp and Woods scale (28). For solubility determination, peptides were dissolved in sterile pure water at 50mg/ml (pH=7), mixed, centrifuged and the presence of a pellet indicated insolubility. Insoluble peptides were sequentially diluted and tested down to 2.5mg/ml or until solubility was observed. Peptides not fully dissolved at 2.5mg/ml were considered insoluble in this study.

Results

T-Cell Epitope Prediction of Alt a 1 Using ProPred Algorithm Server Output

The Barcelona A. alternata allergic patient population used in this study showed a 96% IgE sensitization to Alt a 1 which confirmed the use of this allergen as a target for T-cell epitope prediction and immunotherapy development. Analysis of the complete Alt a 1 sequence including the signal peptide (total 157 amino acids) using ProPred with 11 DRB1 alleles as theoretical binding targets produced 27 potential T-cell epitope 9mers designated by sequence position in Alt a 1 (Tables II & III). Seventeen 9mers had at least one prediction at the higher stringency level and ten 9mers only at the low stringency level. The 7 highest North American-European population frequency DRB1 alleles accounted for 25 of 27 predictions while the addition of the next 4 alleles only produced two additional predictions. Predicted promiscuity of the peptides spanned the full range from 1 to 11 alleles. Twenty-three predicted 9mer epitopes were extended from their C and N termini using flanking Alt a 1 sequence and positioned at p4/p12 within 15mers. Two additional 15mers, located at the N and C termini of Alt a 1 were designed; peptide p1-15 includes the sequences of 9mer peptides p1-9 and p3-11 at positions p1/p9 and p3/p11 respectively and peptide p143-157 which includes the sequences of the 9mer peptides p147-155 and p148-156 at positions p5/p13 and p6/p14, respectively. Considering patient and control cell availability it was decided to proceed with the 25 ProPred derived 15mers for further analysis. Five of these peptides, p67-81, p115-129, p121-135, p124-138 and p135-149 contained a single cysteine residue, one peptide, p1-15, contained a single methionine residue and one peptide, p83-97, contained both a cysteine and methionine residue. Cysteine is subject to oxidation and disulfide bond formation under relatively mild conditions (29) which along with cysteinylation (30) can interfere with peptide MHC binding and activation of T cells by exogenous class II T-cell epitope peptides. Cysteine was substituted with valine as it has similar biochemical properties and has been reported to enhance peptide stability without changing immunological properties (31). Methionine is also sensitive to oxidation and was replaced with biochemically similar leucine to protect against oxidative destabilization (32).

In vitro MHC Binding Assay and ProPred Prediction Evaluation

To confirm ProPred predictions and to validate peptides for continued analysis using IL-4 ELISPOT, an in vitro MHC-peptide binding assay was used to measure binding of 22/25 15mers (excluding p3-17, 42-56, and 43-57) to the 7 DRB1 alleles which accounted for the majority of the ProPred predictions (Table II). For ProPred confirmation, the data showed combined high and low stringency binding prediction rates of 82% (9/11) for allele *0101 and 77% (10/13) rate for allele *0401, with significant false negative rates of 46% (5/11) and 78% (7/9), respectively. High stringency binding prediction rates were accurate for alleles *0301 (none predicted) and *1501 (none predicted), while inaccurate with low stringency positive binding prediction rates of 0% (0/10) and 25% (1/4), respectively. Both alleles *0301 and *1501 had low false negative rates of 8% (1/12) and 6% (1/18), respectively. ProPred was inaccurate at both high and low stringency for the three remaining alleles with a 0% binding prediction rate for *0701 (0/9), *1101 (0/14) and *1301 (0/7) but also yielding 0% false negative rates at 0/13, 0/8 and 0/14, respectively. Of the peptides tested for MHC binding, results showed 1 peptide bound 4 alleles, 1 peptide bound 3 alleles, 10 peptides bound 2 alleles, 7 peptides bound 1 allele and 3 peptides bound 0 alleles. The ProPred prediction method used had an overall one DRB1 allele minimum binding prediction rate of 86.4%. The oxidation stabilizing substitutions did not preclude peptide/MHC binding as all 7 of the substituted peptides bound one or two DRB1 alleles. To conserve patient and control cells, the three non-binding peptides, p35-49, p103-117 and p104-118 were dropped from further analysis leaving a total of twenty-two 15mers for IL-4 ELISPOT analysis.

ELISPOT Data Analysis

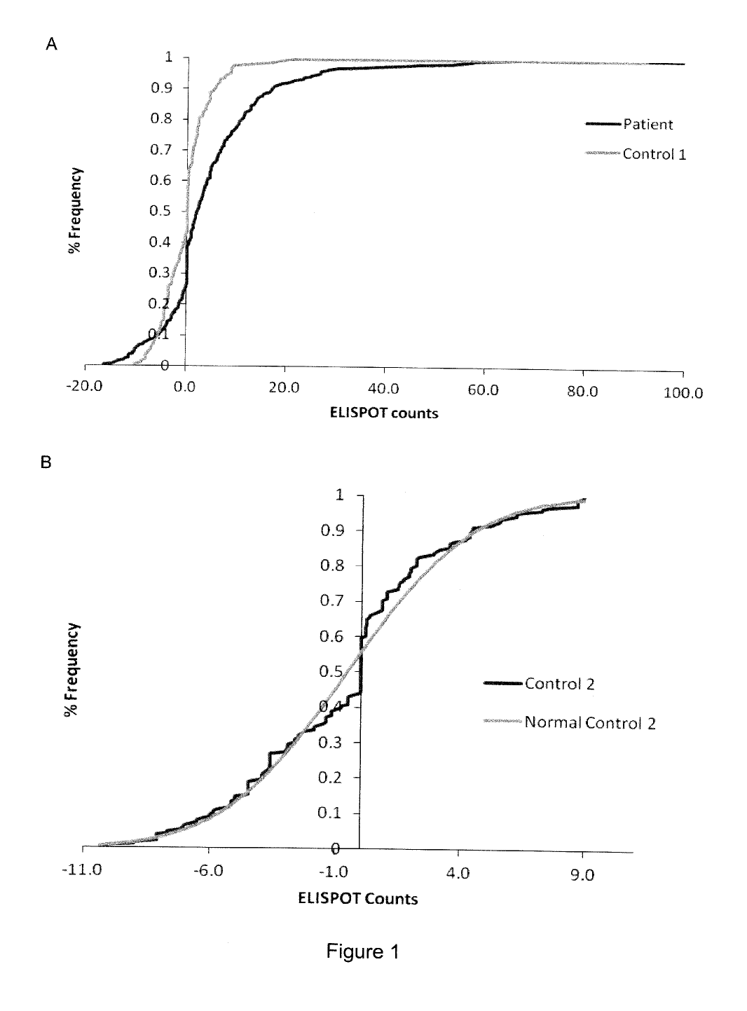

Two methods were evaluated to interpret the IL-4 ELISPOT spot count data. In order to account for assay variability and/or peptide responses found in the control population for quantification of Alternaria allergy specific responses, no-peptide cell background means were subtracted from corresponding control and Alternaria patient peptide means (Table IV) and subjected to hypothesis testing Seven peptides showed statistical significance (p<0.05); p12-26, p51-65, p52-66, p55-69, p59-73, p113-127, and p115-129. However, examination of the data showed that this form of analysis can be insensitive to isolated positive responses and it does not provide any guidance for positive cut-off value determination for peptide/population promiscuity calculations and target population coverage analysis. Since Alternaria negative control group data was available, an empirical approach was used for positive response cut-off determination by plotting the actual cumulative distribution frequencies of the background subtracted control data for each peptide and control subject revealing a non-normal distribution termed control 1 (FIG. 1A); μ=0.29, σ=8.05, median=0.0. However, ≈43% of the control data is at or below 0 spot counts and ≈42% of the data is at or higher than 0 spot counts and the outlier labeling rule identified 4 (g=1.5) to 5 (g=2.2) outliers indicating a potential underlying normal distribution. Cumulative frequencies of background subtracted Alternaria allergic patient data for each peptide and patient showed a non-normal distribution more skewed to the right (FIG. 1A); μ=5.14, σ=12.79, median=2.0 with ≈27% of the patient data at or below 0 spot counts and ≈61% at or higher than 0 spot counts. Removal of the 5 outliers from the control 1 data produced a more normal distribution termed control 2 (FIG. 1B); μ=−0.55, σ=3.93, median =0.0 with deviation in the midsection due to increased 0 counts which comprise ≈16% of the data points. Control 2 data was normalized to model a normal distribution and is termed Normal Control 2 (FIG. 1B). Positive assay cut-off was set at >9.0 spot counts, which was greater than the last control 2 data point and between 2 and 3 standard deviations above the control 2 mean. The five control 1 population peptide counts >9.0 derived from two control subjects were designated as positive responses to peptide. One subject (C8) was IgE positive to two types of pollen with spot counts of 17, 21 and 91.8, while the other subject (C17) was negative for measurable atopy with spot counts of 13.1 and 18.6.

Peptide to Patient Response and Therapeutic Population Coverage

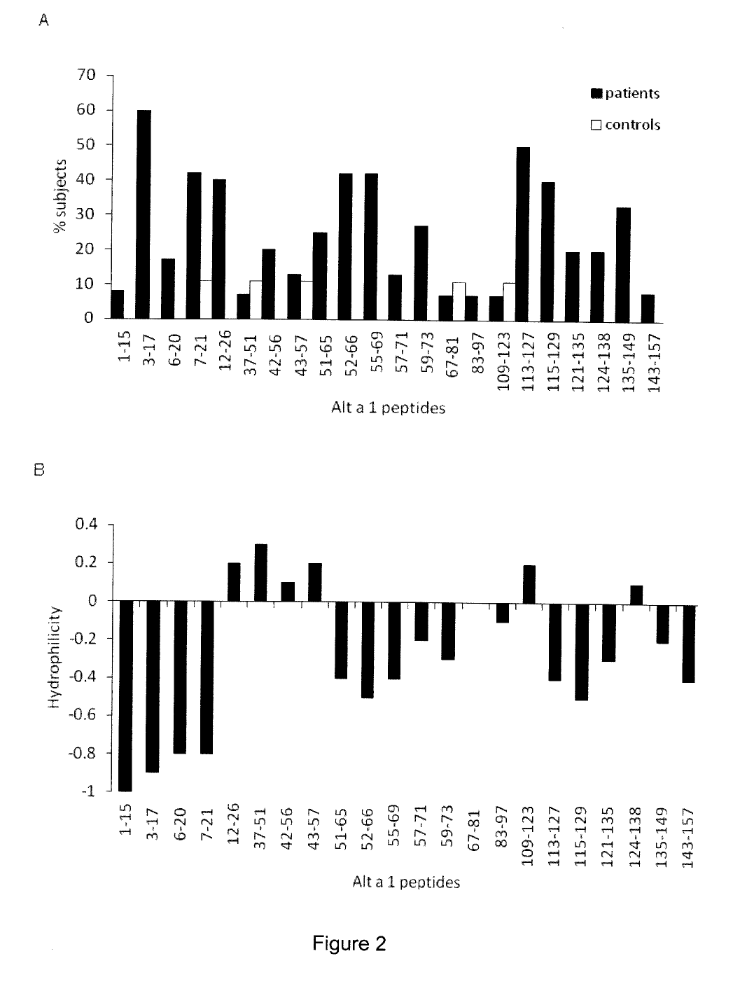

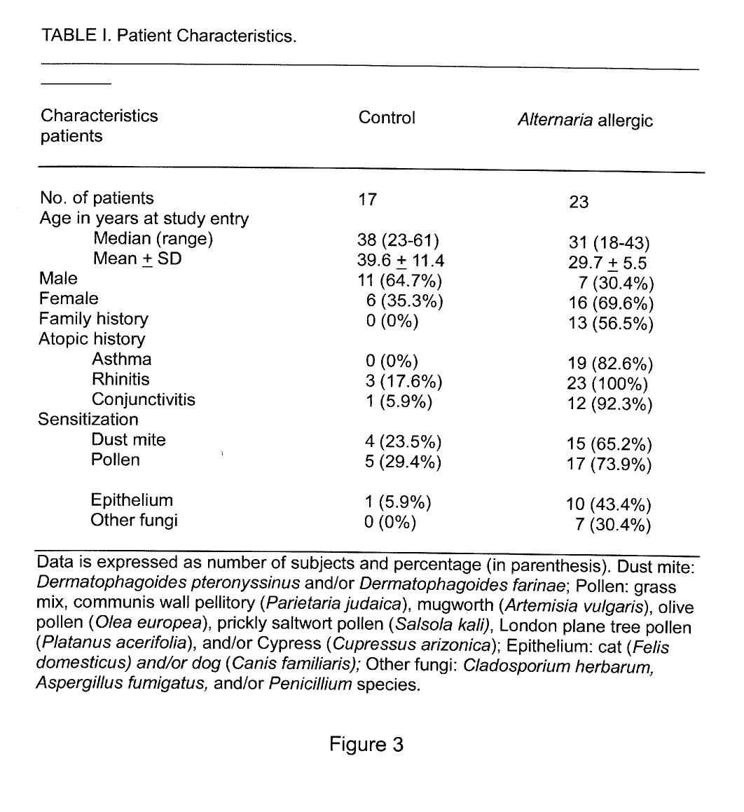

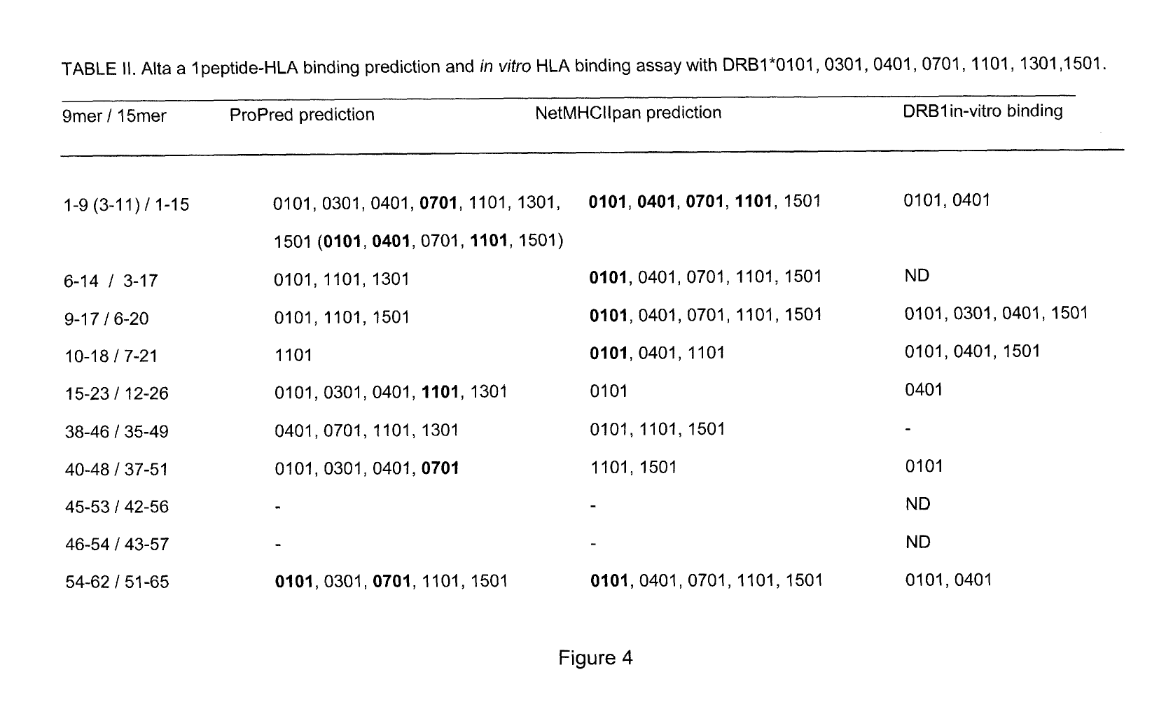

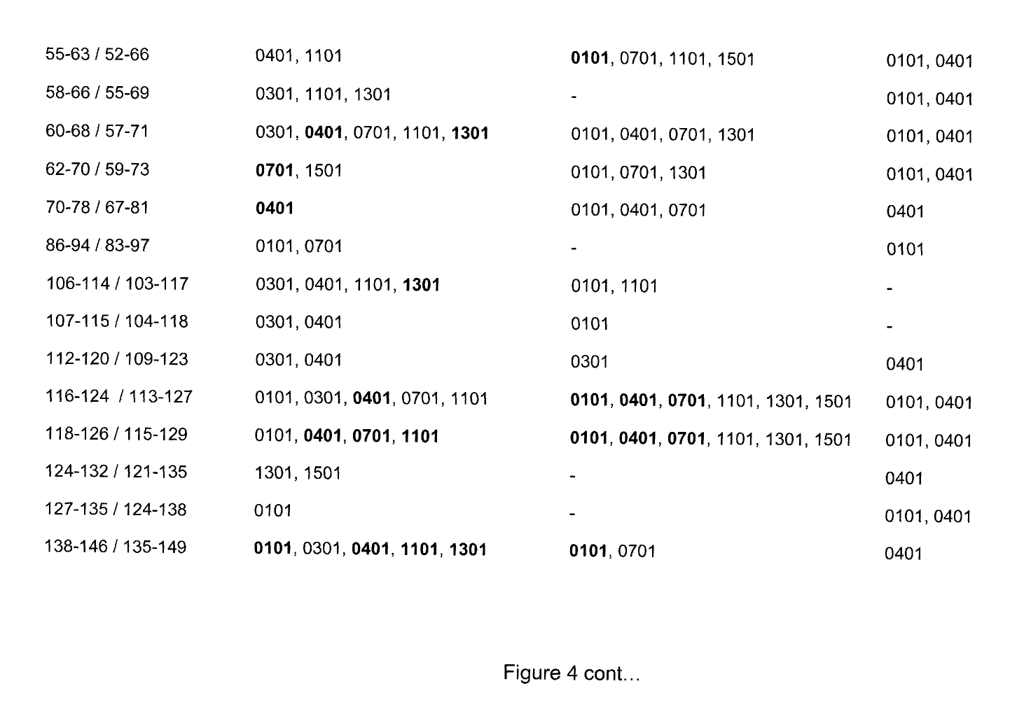

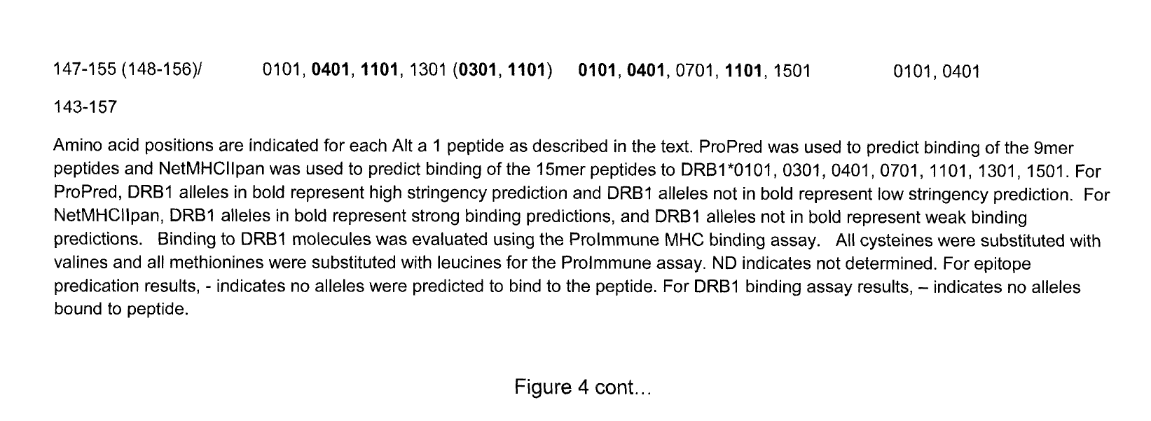

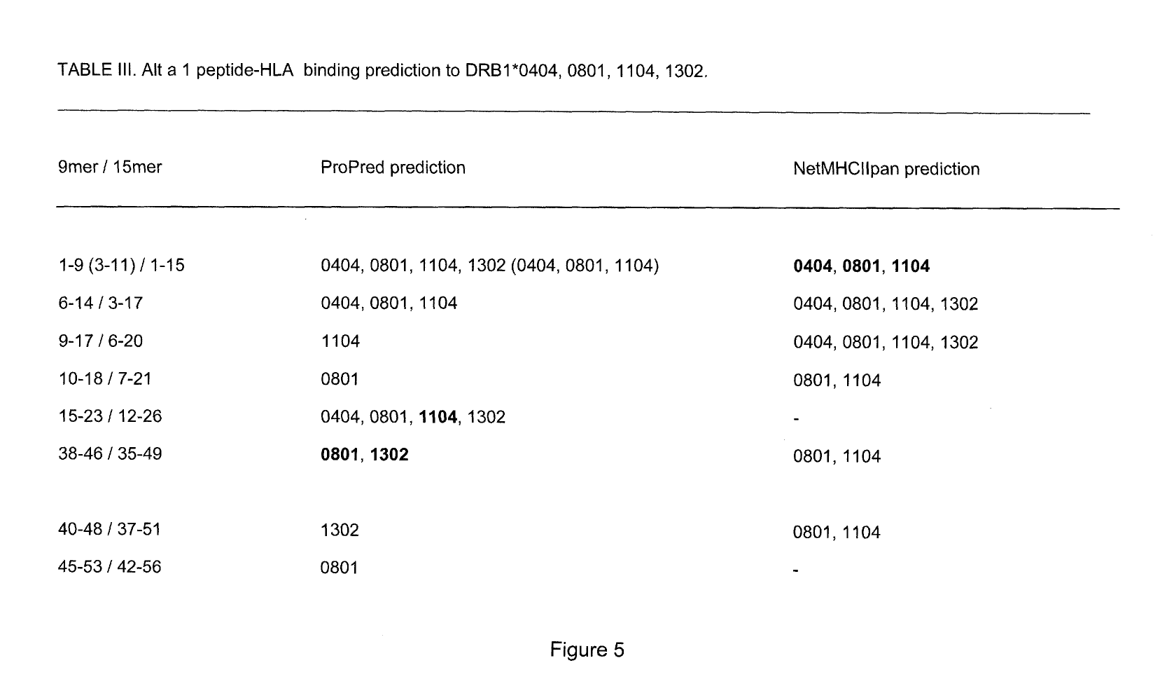

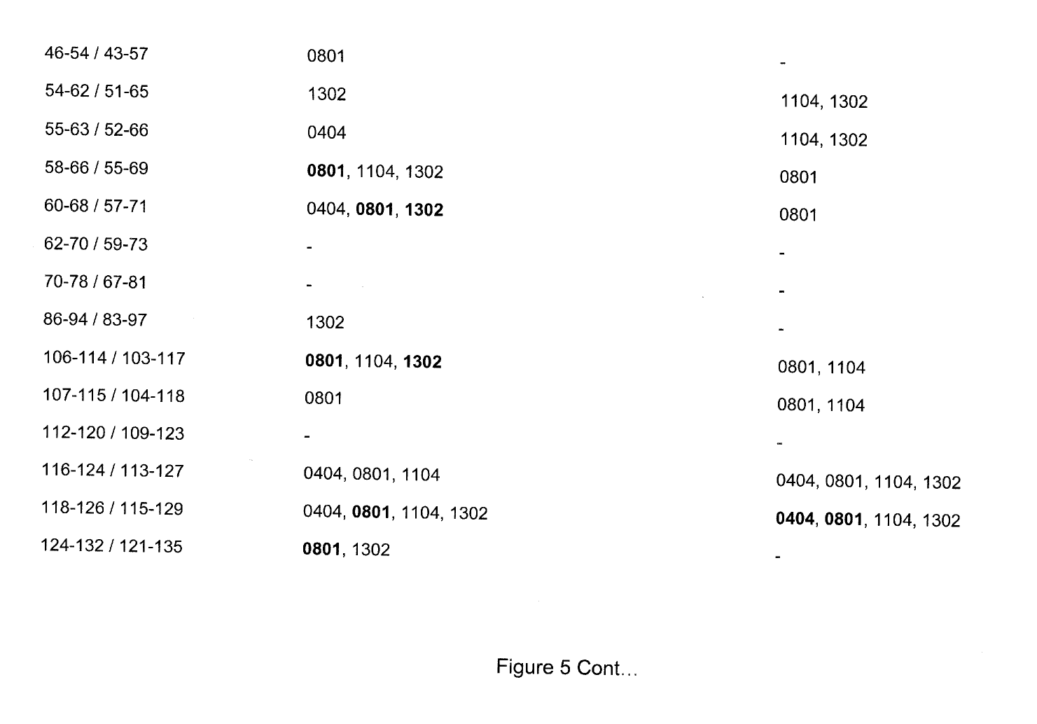

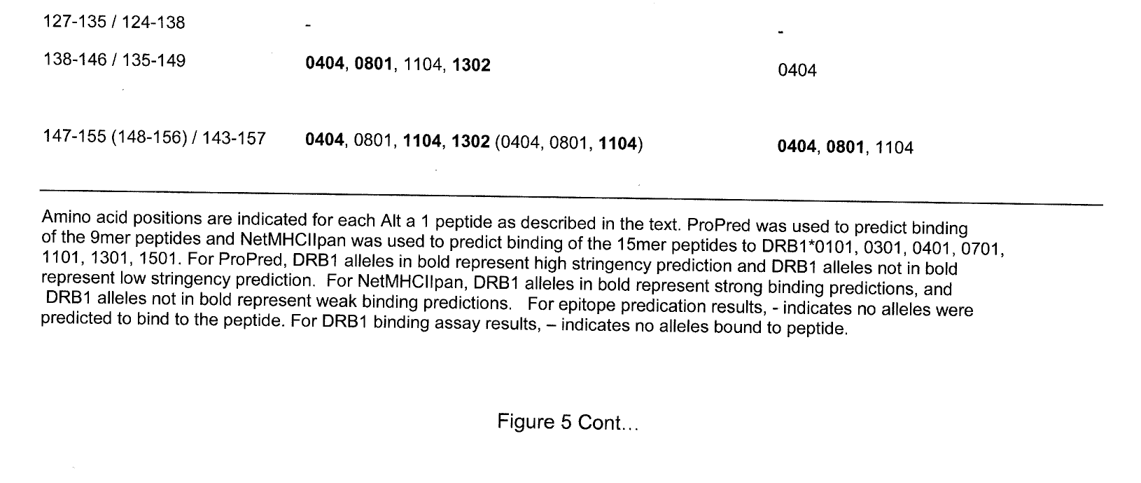

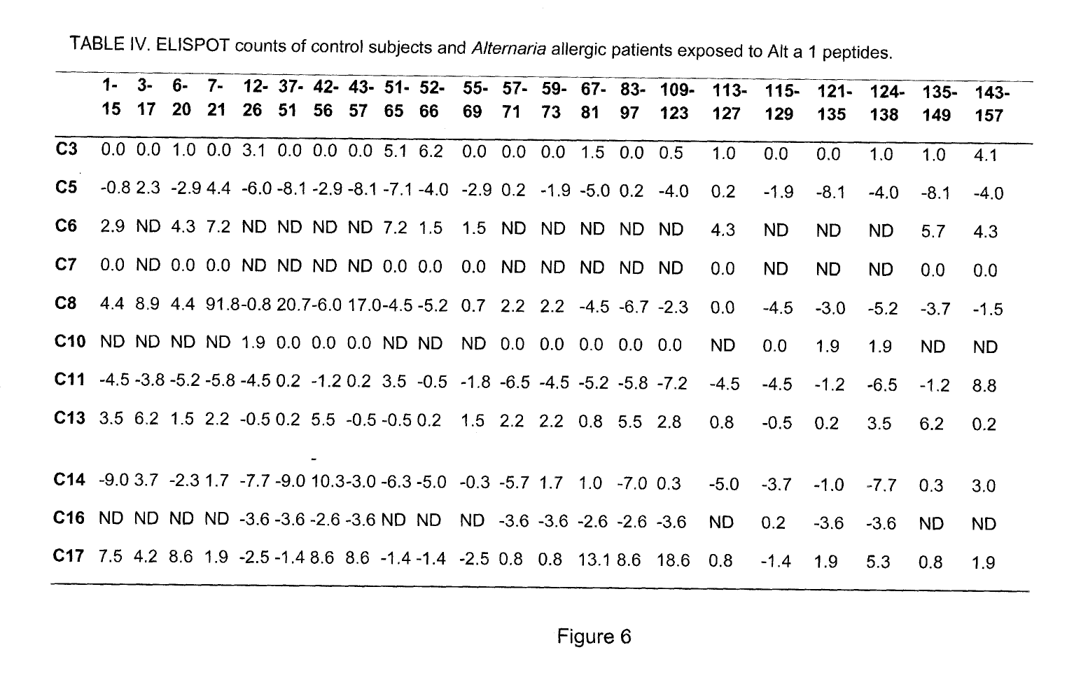

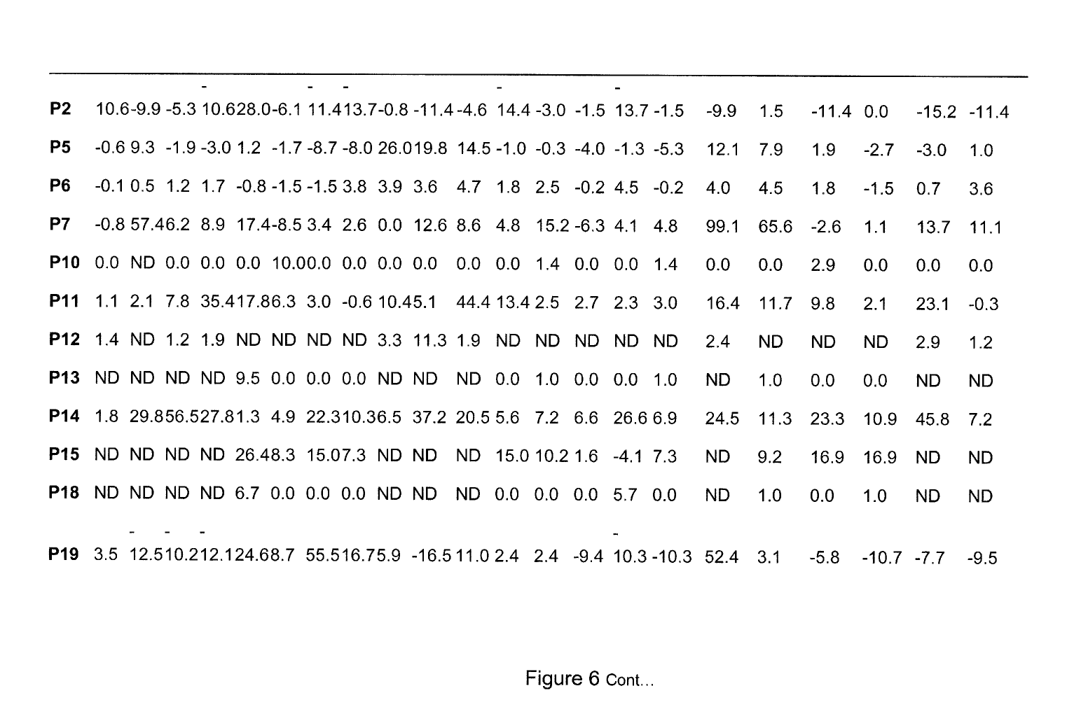

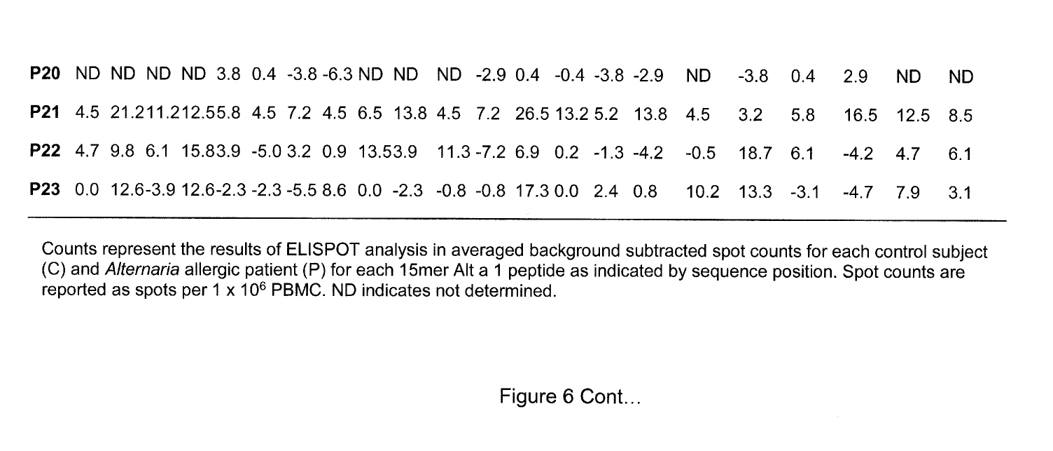

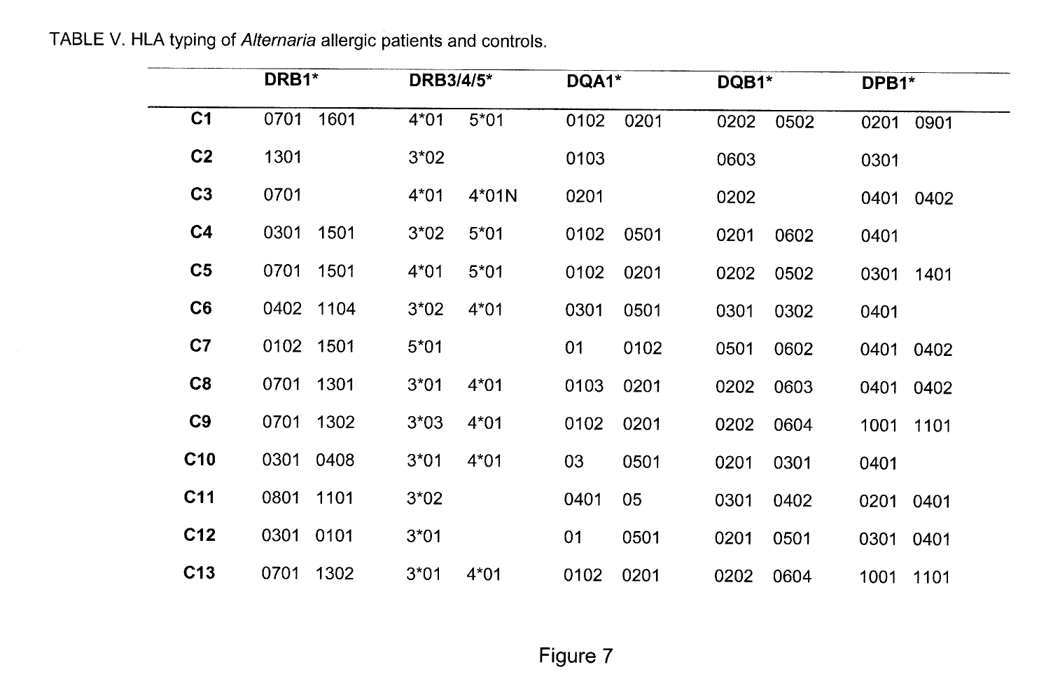

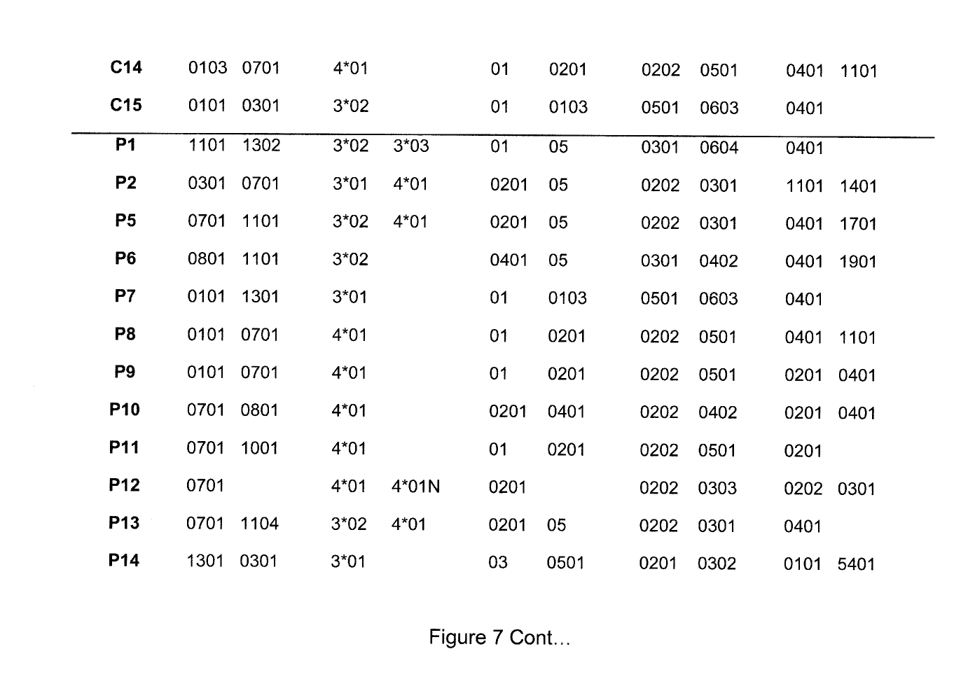

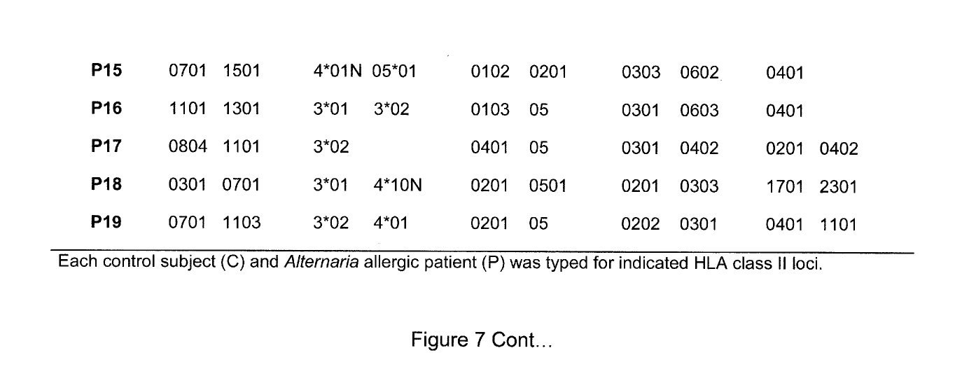

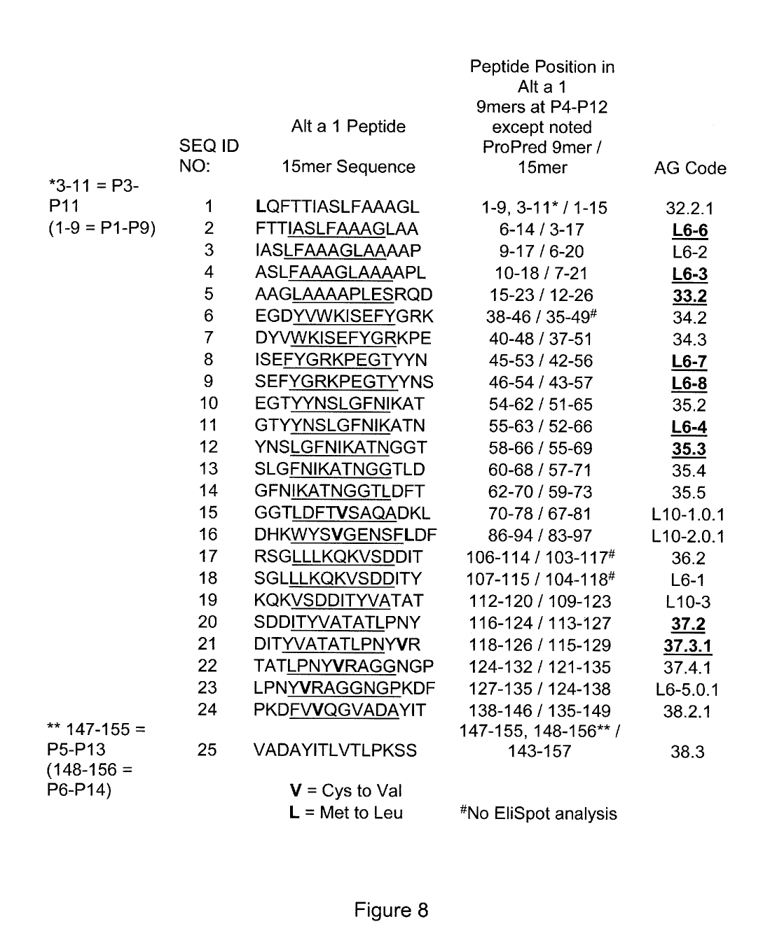

A total of 71 Alternaria patient spots >9 were identified and the % patient response for each peptide was calculated (FIG. 2A). All 22 peptides showed reactivity in at least one patient with a percent tested population response range of 7-60%. The data showed the most promiscuous peptides were concentrated in 4 regions of Alt a 1. Region 1 includes the signal peptide and mature protein N-terminus, region 2 spans residues 51-73, region 3 spans residues 113-129 and region 4 is near the N-terminus including residues 135-149. Sixteen peptides could be considered “promiscuous” by stimulating >8% of their populations, while the top seven of this group (p115-129, p12-26, p55-69, p52-66, p7-21, p113-127, p3-17) showed ≧40% patient reactivity. We then identified subsets of peptides from the ≧40% set that could stimulate the majority of the potential patient population from the 10 patients who were tested with all 22 peptides. These patients showed a wide variation in peptide reactivity. One patient (P6) of this group, who showed detectible IgE reactivity to Alt a 1, was not reactive to any Alt a 1 peptides while one other patient (P2) reacted to 2 peptides, four patients (P5, P19, P22, P23) reacted to 5 peptides, one patient (P7) reacted to 8 peptides, two patients (P11, P21) reacted to 9 peptides, and one patient (P14) reacted to 13 peptides. Using the peptide reaction data corresponding to each individual patient, p12-26 paired with p3-17 would cover 9/10 or 90% of the fully tested patients with at least one reactive peptide. Combinations of p12-26 with 2 other peptides will also cover 90% of the population including: p7-21/p52-66, p7-21/p55-69, p7-21/p113-127 and p52-66/p115-129. Thus, these top seven promiscuous peptides can serve as a pool for peptide immunotherapy in this population and perhaps beyond.

Peptide Hydrophilicity and % Patient Reactivity

Predicted peptide hydrophilicity was plotted and compared to % patient reactivity (FIG. 2B). A Pearson product moment correlation was computed to assess the relationship between peptide hydrophilicity and patient reactivity to peptide. There was a negative correlation between the two variables of r=−0.42, p=0.05, n=22 for all 22 peptides assayed by ELISPOT. Removal of peptides p1-15 and p143-157 increased the negative correlation to r=−0.64, p=0.003, n=20. Thus for the Alt a 1 peptides, decreases in hydrophilicity were correlated with increases in patient reactivity. It is also notable that the peptides which showed no in vitro binding to any DRB1 allele; p35-49, p103-117, and p104-118 were hydrophilic with hydrophilicity calculated at 0.3, 0.4 and 0.1, respectively.

Peptide Solubility

Of the 22 peptides synthesized for ELISPOT analysis, 12 were soluble and 10 were insoluble in H₂O. The insoluble peptides included p1-15, p3-17, p6-20, p7-21, p51-65, p52-66, p55-69, p113-127, p135-149, and p143-157. As expected, solubility was broadly associated with predicted hydrophilicity; peptides ≦−0.8 were insoluble, peptides ≧−0.1 were soluble, while peptide solubility was variable in the intermediate range. As hydrophilicity was negatively correlated with % patient reactivity, it is not surprising that 5 of the 7 peptides with ≧40% patient reactivity were insoluble. It is of possible interest to produce and assay water soluble peptides for use in immunotherapy or diagnostics, therefore a subset of 6 insoluble peptides were modified by single amino-acid changes at or near the N-terminus and then retested for solubility. The following modified peptides with calculated hydrophilicity were soluble in H₂O: p51-65:G52S (−0.3), p55-69:Y55A (−0.3), Y55S (−0.2), Y55E (0.0), p143-157:V143S (−0.3), V143E (−0.1), and p52-66:G52S (−0.5). The following modified peptides were insoluble in H₂O: p51-65:G52E (−0.2), p55-69:Y55V (−0.3), p113-127:S113E (−0.2), p135-149:P135S (−0.2), P135E (0.0), p143-157:V143A (−0.3), and p52-66:G52E (−0.3). While most of the substitutions increased the calculated hydrophilicity of the peptides it was not necessarily associated with improvement in solubility nor was there any pattern in the residues used for substitutions. However, out of 14 modified peptides tested, 7 showed improved solubility while of the 6 original insoluble peptides targeted for modification, 4 peptides had at least one soluble variant.

Comparison of Population DRB1 Typing Data, Peptide in vitro Binding and Patient Reactivity

To facilitate the determination of the potential patient DRB1 alleles involved in patient reactivity to peptides, the population percentage of patient and control subjects either homo- or heterozygous for each DRB1 allele was determined (Table V) and compared to the in vitro DRB1 binding data (Table II) and patient peptide reactivity (Table IV). The results of the DRB1 binding assays showed that the majority of the peptides bound to the *0101 and *0401 allele proteins. However, none of patient population was bearing the *0401 allele while 18% had the *0101 allele. Of the ELISPOT tested patients, one heterozygous for the *0101 allele (P7) was reactive to five *0101 binding peptides. For the remaining alleles, one patient (P14) who was heterozygous for the *0301 allele was reactive to a single *0301 binding peptide. No other concurrences were present between reactive patient DRB1 alleles and peptides with matching DRB1 binding. The remaining relevant DRB1 alleles in the patient population were *0701 at 65%, *1101 at 29% and *1301 at 18%. However, the DRB1 binding assay showed no positive peptides for these three alleles. While the possibility exists for technical issues with the binding assay, it is more likely that the peptides are binding MHC molecules from other class II loci. DQB1 typing was performed to determine if other HLA loci are potential participants in the peptide presentation (Table V). DQB1 typing showed 2 alleles of interest; *0202 was the most abundant in both patients and control populations while *0301 was present in 47% of the patients but only 20% of the controls. Binding predictions using NetMHCII 2.2 server could be obtained for a limited number of DQB1 alleles. Binding predictions of Alt a 1 peptides to DQA1*0501-DQB1*0301 included strong binders in region 1 and weak binders for regions 2, 3 and 4 suggesting that significant peptide presentation could occur through loci other than DRB1.

Retrospective Analysis of NetMHCIIpan-2.1 Server Epitope Prediction Algorithm Output and MHC Binding Assay Results

For a comparison of ProPred results with a prediction server based on a different method, NetMHCIIpan-2.1 was used to calculate default level weak and strong binding predictions to the 11 most common DRB1 alleles for all 143 15mers present in the complete Alt a 1 sequence. Evaluation of allele specific NetMHCIIpan binding predictions utilizing the in vitro binding results for the 22 15mers (Table II) revealed combined strong and weak binding prediction rates of 63% (10/16) for allele *0101(<ProPred), and a 100% (9/9) rate for allele *0401, (>ProPred) and similar to ProPred with significant false negative rates of 67% (4/6) and 62% (8/13) respectively. The strong binding prediction rate was accurate for alleles *0301 (none predicted) and *1501 (none predicted) which was similar to ProPred's high stringency predictions but was more accurate than ProPred for allele *1301 (none predicted). The weak binding prediction of one false positive for *0301 was also more accurate than the ProPred low stringency prediction but similar to ProPred with poor weak binding prediction for allele *1301 and *1501 at 0% (0/4) and 11% (1/9), respectively. All three alleles *0301, *1301 and *1501 had low false negative rates of 5% (1/21) 0% (0/18) and 7% (1/15), respectively. Like ProPred, NetMHCIIpan was inaccurate at both weak and strong binding prediction for the two remaining alleles with a 0% binding prediction rate for *0701 (0/11) and *1101 (0/11) but also yielded 0% false negative rates at 0/11 and, 0/11 respectively. In conclusion, NetMHCIIpan had lower false positive rates compared to ProPred for two alleles but was similar to ProPred in other binding predictions and most importantly for the two alleles responsible for the vast majority of the positive in vitro binding results.

Retrospective Analysis of ProPred/NetMHCIIpan Prediction and Peptide Response Results

NetMHCIIpan using the same 11 DRB1 alleles as ProPred predicted in Alt a 1 a total of 27 strong binders to at least one allele, of which 2 were ranked as strong binders only while the remaining 25 were also ranked as weak binders for other alleles. An additional 53 peptides were ranked as weak binders only, bringing to a total of 80 unique peptides ranked as binders. The strong binders were distributed primarily in the same 4 high reactivity regions identified with ELISPOT analysis of ProPred predictions. For the 25 ProPred derived 15mers used in this study (Tables II & III), NetMHCIIpan predicted 10 peptides as strong binders for at least one allele, 9 peptides as weak binders only and 6 peptides were not predicted at all. For the seven ELISPOT assayed peptides with a ≧40% patient response, NetMHCIIpan predicted 5 peptides as strong and weak binders to multiple alleles while the remaining two peptides only had one weak prediction each from all 11 DRB1 alleles. Similar results were seen with ProPred predictions for the same 7 peptides with 5 peptides predicted at high stringency and 2 peptides with only 2 or 3 low stringency predictions. The clearest reactivity prediction failure of ProPred/NetMHCIIpan were peptides p1-15 and p143-157 both of which had high stringency/strong binding predictions to multiple alleles but only stimulated one patient each. It is notable that these peptides were the first possible N-terminal and last possible C-terminal 15mer peptides. A Pearson product moment correlation was computed to assess the relationship between predicted peptide promiscuity and patient reactivity to peptide. Positive Pearson correlations between the totaled number of predictions per peptide of ProPred/NetMHCIIpan for 11 alleles and % patient response per matching peptide for all 22 ELISPOT peptides were r=0.29, n=22, p=0.18 for ProPred and r=0.34, n=22, p=0.12 NetMHCIIpan. Removal of peptides p1-15 and p143-157 increased the correlation to r=0.48, n=20, p=0.03 for ProPred and r=0.51, n=20, p=0.02 for NetMHCIIpan. Thus for the Alt a 1 peptides, increases in predicted peptide promiscuity were correlated with increases in patient reactivity.

Discussion

Early therapeutic design strategies for peptide immunotherapy for allergy were not typically focused on the identification and specific use of relevant T-cell epitopes. These strategies utilized long peptides/fragments (>20 residues) partially or completely covering the target allergen or smaller (<20 residue) partially overlapping peptides covering the entire allergen (17, 33, 34, 35). For a T-cell epitope based strategy, a direct approach to completely screen even a small allergen such as Alt a 1 would require 143 15mer peptides and thus more economical screening strategies have been reported (36, 37). A strategy utilizing a set of 15mers overlapping every five residues would limit an Alt a 1 screen to ≈29 peptides, however our results showed large differences in patient reactivity by shifts of one residue, so while this method may identify regions of reactivity, additional regional peptide mapping would be required to produce an optimized peptide mix, incurring further expense and increased patient sampling. The production of T-cell lines by expansion with whole allergen or peptide can provide a source of cells for further analysis and has been shown to be effective in T-cell epitope identification and can yield population coverage data (19). However, this method will not provide an accurate quantitation of specific memory T-cell populations for determination of clinically relevant peptide reactivity for peptide immunotherapy development and differential activation could also alter count proportions between peptides.

For therapeutic development our study tested the combination of in silico epitope prediction and in-vitro MHC binding with direct PBMC peptide stimulation measured with the very sensitive cytokine specific ELISPOT assay. The potential advantages of in silico prediction has been described (38, 39, 40) and this approach has been utilized in a number of allergen T-cell epitope identification projects including peptide immunotherapy development (19, 35, 41, 42, 43). Currently available T-cell epitope prediction servers for Class II binders are primarily based on three different methodologies; quantitative matrices including the original TEPITOPE DRB1 virtual pocket profile matrix as used in ProPred (44), support vector machines such as MHC2Pred, and binding data driven methods, including NetMHCIIpan which uses artificial neural networks for peptide/MHC binding affinity based prediction for DRB1 alleles (38, 40). Analysis has shown several software packages including NetMHCIIpan outperforming ProPred (38), and indeed, our study showed ProPred with a higher false positive prediction rate for several alleles compared to NetMHCIIpan, however, this had little effect on the overall similar predictive ability of ProPred and NetMHCIIpan for our peptide set due to the pooling of a large number of alleles to enhance promiscuous epitope prediction. ProPred was suitable for our study as it predicted in total our target of 20-30 peptides spanning multiple regions in Alt a 1 and produced a set of highly reactivity peptides in Alternaria patients, but it is highly probable that the NetMHCIIpan strong binder predictions would produce a comparably sized high coverage peptide mix. In addition, unlike ProPred which only reports the top 10% binding predictions (38), the NetMHCIIpan method allows complete predictive mapping of the entire allergen, defines binding regions for expanded analysis, predicts more binders than ProPred for larger scale mapping projects, predicts more DRB1 alleles and is more accurate for certain specific allele predictions. A recently upgraded server based on the TEPITOPE matrices, TEPITOPEpan, claims a significant increase in DRB1 allele coverage and overall performance, although second to NetMHCIIpan overall, TEPITOPEpan was superior in binding core recognition (45). Our analysis also confirms that ProPred and NetMHCIIpan, while both exclusively DRB1 prediction servers, are sufficient to generate promiscuous multi-loci class II epitopes.

In our study we utilized the cytokine specific ELISPOT assay to measure Th2 T-cells induction by peptides as the ³H-tritium incorporation method is not a specific indicator of a Th1 or Th2 phenotype. The ELISPOT technique has emerged as a primary tool in the clinical monitoring of vaccine trials and other forms of immunotherapy (46). ELISPOT based clinical assays for the measurement of INF-γ from Th1 CD4+ and CD8+ T-cells activated by specific well-characterized peptides have lead standardization efforts in assay optimization to lower signal-to-noise ratio and to improve data analysis (47). Data analysis theory has centered on developing criteria for identifying positive immune responses from ELISPOT data by comparison of peptide containing wells to media only (no peptide) control wells using empirical rules such as certain fold changes above control or statistical evaluations (48, 49). Recent recommendations for comparison of peptide and non-peptide wells favor statistical analysis using various parametric and non-parametric hypothesis testing procedures as well as rigorous data rejection criteria which may be difficult to apply in situations with limited cell numbers and sub-optimized assays with multiple peptides. In our study, which included the use of serum-free media and standardized procedures for the preparation of frozen PBMC, the use of well-characterized disease and control populations with positive response cut-off determinations allowed clear interpretation of IL-4 ELISPOT data for a CD4+/Th2 T-cell epitope discovery project.

While our study is the first report of a potential pool of Alt a 1 peptides for high population coverage peptide immunotherapy, a previous study by Oseroff et al. (19) tested 7 Alternaria peptides using epitope prediction and IL-5 and INF-γ ELISPOT with an allergic population and reported 6 peptides as IL-5 positive. Three of the peptides were identical 15mers to peptides tested in our study, including p1-15 reported as negative to both cytokines, p6-20 reported positive for IL-5 only and p143-157 also positive for IL-5 only. These results provide confirmation of the low level of patient reactivity for the N-terminus peptide p1-15 despite its highly promiscuous MHC binding prediction and in-vitro MHC binding. They also reported 4 additional Alt a 1 peptides, 3 of which were both positive in atopic subjects for IL-5 and INF-γ production indicating a possible mixed Th1-Th2 response for some peptides. One potential complication of this analysis was the use of T cells expanded for 14 days with allergen extract and IL-2 in which the polarization of naïve T cells could be skewed via bystander effects from polarized memory T cells. It has been shown that limited N-terminal degradation of an exogenous Class II peptide by dendritic cells blocked MHC binding but was preventable by N-terminal modification (50), although typical short time frame PBMC based ELISPOT assays do not generate monocyte derived dendritic cells. This observation suggests a possible mechanism for the poor reactivity of select peptides such as p1-15 and p143-157 in our study. However, it may be more likely that the N and C-terminal positions of p1-15 and p143-157 in the intact Alt a 1 allergen may promote sequence loss due to endolytic degradation of the whole allergen prior to or during processing by antigen presenting cells resulting in a lack of presentation of intact versions of these peptides to T-cells.

The extent of CD4+ T-cell reactivity to Alternaria allergen derived Class II T-cell epitopes in normal non-allergic subjects is largely unknown. Extensive Th1 T-cell activation after exposure to Aspergillus fumigatus whole antigens has been observed in a majority of normal subjects (51). Similarly, ELISPOT assays measuring both Th1 and Th2 activation showed that whole A. fumigatus allergens also extensively activated Th1 CD4+ and CD8+ T-cells (52). Both of these results have been interpreted as active innate defense to prevent invasion by an opportunistic pathogenic fungus. While A. alternata can be an opportunistic pathogen in immunosuppressed patients in rare occasions (53), it is primarily associated with allergic disease so the presence in our study control population of atopic and non-atopic subjects with IL-4 T-cell reactivity to Alta a 1 but without Alternaria allergy could be interpreted as an ongoing response to Alt a 1 exposure but balanced by peripheral tolerance blocking production of IgE to Alt a 1. Oseroff et al. (19) assayed predicted T-cell peptides from multiple fungal allergens and showed overall polarization of the A. fumigatus T cell responses to Th1 while Alternaria showed polarization to Th2. A feature of the epitope prediction software servers was the high number of predicted epitopes present in the signal peptide of the Alt a 1 secreted protein. Analysis of five predicted Class II DRB1 binding peptides derived fully or partially from the epitope dense signal sequence of Alt a 1 produced a wide range of responses demonstrating sequence and allergic disease specificity. The N-terminus of the Alt a 1 allergen harbors a predicted signal peptide (predicted to be cleaved between amino acid residues 19-20 by Signal P 3.0) that is most likely cleaved in the fungus and may be retained in the endoplasmic reticulum or secreted during the spore germination process. T-cell activation by signal sequences via Class II MHC has been previously reported in cockroach, peanut and Alt a 1 allergens (42, 43, 19) Similar findings have been reported for Class I epitopes present in signal peptides (54), however, while standard mechanisms for the processing of self or viral proteins could account for signal peptide derived epitope loading onto class I molecules, presentation of exogenous signal peptide derived epitopes by class II molecules may require a dynamic interaction with antigen presenting cells (APCs) and Alternaria spores or hyphae, possibly related to the degradation stability of cleaved signal peptides (55) or the presence of Alt a 1 pre-protein isoforms and the kinetics of phagosome digestion of spores/germinating spores and hyphal fragments (56. These observations also suggest that the use in models and assays of processed mature versions of secreted allergens for sensitization or T-cell stimulation may result in less accurate descriptions of the allergic process under study. More work will be required in the future to determine the localization of this signal sequence portion of Alt a 1 within the fungus itself or following the secretion process.

While a small sample size, HLA typing showed more than a doubling in the frequency of DQB1 allele *0301 in Alternaria allergic patients compared to the controls. The DQB1 *0301 allele is one of several *03 alleles which have been reported as risk factors for allergic fungal rhinosinusitis (AFRS) (57). Patients with AFRS usually have a history of atopy and allergic rhinitis as do all of the Alternaria allergic patients in our study group. AFS is typically associated with the isolation of a number of fungal species from the allergic mucin most commonly A. fumigatus and dematiaceous species including A. alternata with no evidence of invasive disease (58). An association of Alternaria allergy and the DQB1*03 alleles suggests a possible genetic predisposing mechanism of initial induction of fungal atopy and rhinitis by Alt a 1 and expansion via epitope spread leading to sensitization to other fungal species through conserved allergens followed by development of sinusitis in a subset of patients. Further investigation will be required to validate the aspects of this proposed mechanism.

Despite wide variations in individual patient T-cell reactivity, a core group of seven peptides accounted for the majority of the reactivity, it is possible for as few as 2 of these peptides to be recognized by 9/10 Alternaria allergy subjects. As the presentation of these promiscuous peptides likely occurs through multiple alleles from 2 or more loci (HLA DR, DQ and DP), the potential exists for broad coverage between geographical populations. For example, while the Barcelona population showed some differences, the 7 highest frequency DRB1 alleles and the 5 highest DQB1 alleles from the North American European American population also contained the top 5 and 3 alleles, respectively, of the Barcelona population.

Of interest for potential peptide immunotherapy is the presence of some patients non-responsive to Alt 1 peptides who nevertheless have significant levels of IgE to the Alt a 1 allergen. Similar findings have been reported in a multi-allergen study of cockroach allergic patients following screening with large numbers of predicted T-cell epitope peptides (42). While potential reactivity in such negative patients to additional untested peptides cannot be ruled out in these cases, a large heterogeneity of patient/peptide responses is evident and points to multiple pathways of CD4+ T-cell activation leading to specific IgE production possibly linked to MHC restriction and/or a temporal evolution of the allergic responses. Also of interest for peptide immunotherapy development would be any disconnection between T-cell epitope reactivity and IgE to the corresponding allergen as well as the lack of a dominant allergen for population coverage, thereby necessitating multi-allergen peptide mixtures all leading to increased development time, expense and sampling ethical concerns (42). However, in our study of Alt a 1, the impact of the above issues has been minimal and more similar to Fel d 1 for cat allergy. Alt a 1 appears to be an excellent candidate for a single allergen based T-cell epitope peptide immunotherapy for treatment of Alternaria allergy.

Peptides identified during screening and used in animal models may be soluble and stable in the typical DMSO solutions used in such projects, but may possess chemical and physical properties that lead to formulation issues in preparation for clinical trials. These properties include oxidation of sensitive amino acids such as cysteine and methionine and peptide aggregation due to disulfide bond formation. The use of excipients such as antioxidants and reducing agents is one option for these formulation and delivery issues (59) and has been used to prevent peptide aggregation due to disulfide bond formation in a Fel d 1 based peptide immunotherapy treatment for cat allergy (35). Another option is substitution of sensitive residues with similar but oxidation resistant residues, for cysteine replacement this includes the structural analog serine (29, 37) and in our study the chemical analog valine, both of which have shown to allow retention of immunological activity but simplify formulation and delivery.

In regard to other peptide physical properties, a potential disadvantage of epitope prediction methods would be the introduction of bias due to limitations of the underlying data. It has been noted, and is consistent with our study, that the current methods tend to predict peptides of low hydrophilicity (38), the presence of which can impact therapeutic formulation and delivery. While aqueous soluble peptides may simplify formulation for parenteral administration, low hydrophilicity peptides could open up alternative delivery routes and systems (60). Also, our study showed that single N-terminal residue substitutions can improve solubility of many T-cell epitope containing peptides. This approach and solubility screening of peptides mixes with approved formulation excipents should be able to reduce peptide solubility issues.

Another concern for peptide immunotherapy is potential B-cell epitopes present within peptides which can cross-link IgE leading to immediate hypersensitivity reactions, although this can be tested prior to administration, the induction of treatment induced peptide specific IgE is still an issue. While most B-cell epitopes are conformational (discontinuous), linear (continuous) epitopes are also found and can range from 3-38 amino acids in length with the majority ≦21 amino acids (61). Natural class II peptides have been shown to range from 7-25 amino acids (62) with the most abundant species ranging from 14-21 amino acids (63) and could potentially function as linear B-cell epitopes. A clinical trial of a Fel d 1 based peptide immunotherapy using two 27mer peptides in escalating doses up 750 ug was associated with primarily late phase adverse events but 15% of patients developed IgE to these peptides during the course of treatment (34). It is possible that these longer peptides could form conformational epitopes so the potential for IgE reactivity to linear epitopes present in shorter peptides remains unclear. However, next generation Fel d 1 peptide immunotherapy utilizing shorter 13-17mer peptides and a lower dose has a much improved safety record (35). Screening of peptides used for immunotherapy with linear B-cell epitope prediction servers may offer some insight (64). Peptide length may also influence peptide reactivity as residues added to the 9mer class II binding core peptide have been positively correlated with an increase in predicted MHC-peptide binding affinity with the potential maximum reached at 18-20 residues (65), however, affinity gains decrease sequentially. In addition, N and C-terminus peptide flanking regions outside the core class II 9mer have been shown to have considerable influence on binding of specific T-cell receptors with the peptide-MHC complex (66, 67). The addition of N and C-terminus peptide flanking regions of three residues each appears sufficient to account for the required T-cell receptor peptide-MHC binding affinity. In our study, short 15mer peptides were chosen to minimize the risk of potential B-cell epitopes, retain near optimal affinity, provide defined high specificity, and to reduce treatment production costs.

Peptide immunotherapy has been reported to be safe and effective for the treatment of specific allergies. Our results demonstrate the potential of the T-cell epitopes derived from the Alt a 1 allergen for development into specific therapeutics for the treatment of fungal allergy patient populations. We also have shown the effectiveness of T-cell class II epitope prediction and the IL-4 ELISPOT assay for peptide immunotherapy discovery projects. Filamentous fungi and their unique and conserved allergens represent exciting targets for new types of immunotherapy.

### Example 2

### Characterization and Selection of a Novel T-Cell Epitope of the Major Alternaria alternata Allergen Alt a 5 for Peptide Immunotherapy

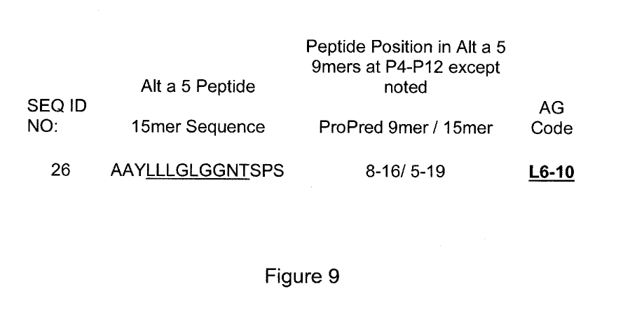

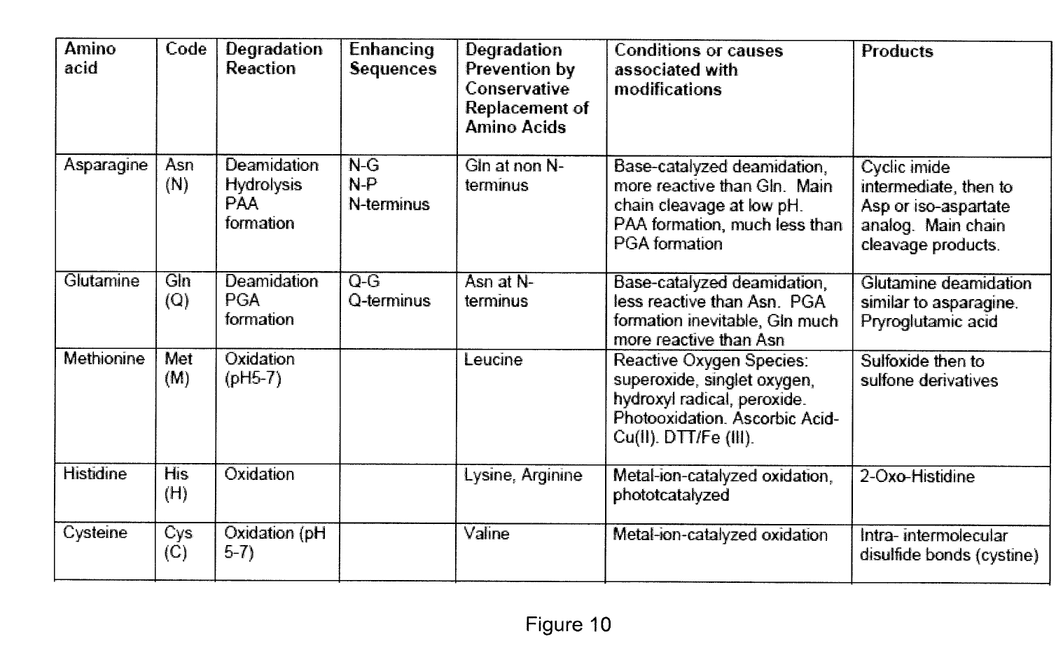

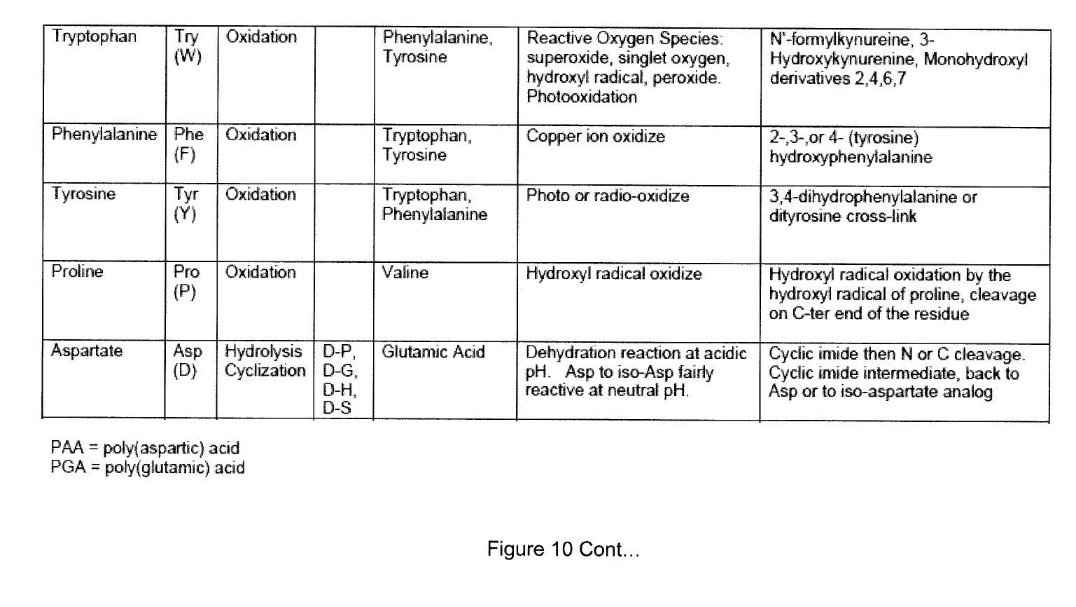

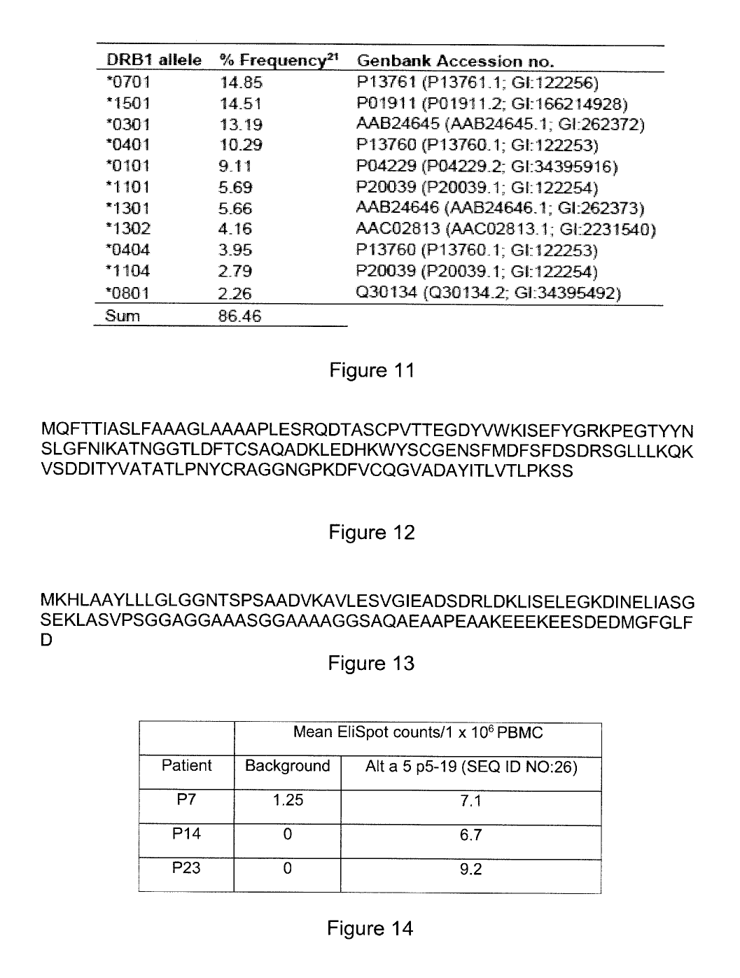

The methodology described above in respect of Example 1 was applied to the Alternaria alternata antigen Alt a 5. This resulted in identification of the novel T-cell epitope p8-16/5-19 (FIG. 9).

Three Alternaria patients exposed to a number of individual peptides from several Alt a allergens were fully tested with the peptide p5-19 (SEQ ID NO:26; FIG. 9) in accordance with the materials and methods described for Example 1 above. The peptide was active in all 3 patients, see results in FIG. 14.

References

1. Bush R K, Prochnau J J. Alternaria-induced asthma. J Allergy Clin Immunol 2004; 113: 227-34.

2. Pulimood T B, Corden J M, Bryden C, Sharples L, Nasser M. Epidemic asthma and the role of the fungal mold Alternaria alternata. J Allergy Clin Immunol 2007; 120: 610-7.

3. Denning D W, O'Driscoll B R, Hogaboram C M, Bowyer P, Niven R M. The link between fungi and severe asthma: a summary of the evidence. Eur Respir J 2006; 27: 615-26.

4. Heinzerling L, Frew A J, Bindslev-Jensen C, Bonini S, Bousquet J, Bresciani M, Carlsen K H, van Cauwenberge P, Darsow U, Fokkens W J, Haahtela T, van Hoecke H, Jessberger B, Kowalski M L, Kopp T, Lahoz C N, Lodrup Carlsen K C, Papadopoulos N G, Ring J, Schmid-Grendelmeier P, Vignola A M, Wöhrl S, Zuberbier T. Standard skin prick testing and sensitization to inhalant allergens across Europe—a survey from the GALEN network. Allergy 2005; 60: 1287-1300.

5. Zureik M, Neukirch C, Leynaert B, Liard R, Bousquet J, Neukirch F; European Community Respiratory Health Survey. Sensitisation to airborne moulds and severity of asthma: cross sectional study from European Community respiratory health survey. BMJ 2002; 325: 411-4.

6. Bousquet P J, Chinn S, Janson C, Kogevinas M, Burney P, Jarvis D, European Community Respiratory Health Survey I. Geographical variation in the prevalence of positive skin tests to environmental aeroallergens in the European Community Respiratory Health Survey I. Allergy 2007; 62: 301-9.

7. Barta J, Belmonte J, Toress-Rodriguez J M, Cistero-Bahima A. Sensitization to Alternaria in patients with respiratory allergy. Front Biosci 2009; 14: 3372-9.

8. Salo P M, Arbes S J, Sever M, Jaramillo R, Cohn R D, London S J, Zeldin D C. Exposure to Alternaria alternata in US homes is associated with asthma symptoms. J Allergy Clin Immunol 2006; 118: 892-8.

9. Lizaso M T, Martinez A, Asturias J A, Algorta J, Madariaga B, Labarta N, Tabar A I. Biological standardization and maximum tolerated dose estimation of an Alternaria alternata allergenic extract. J Investig Allergol Clin Immunol 2006; 16: 94-103.

10. Lizaso M T, Tabar A I, García B E, Gómez B, Algorta J, Asturias J A, Martínez A. Double-blind, placebo-controlled Alternaria alternata immunotherapy: in vivo and in vitro parameters. Pediatr Allergy Immunol. 2008; 19: 76-81.

11. Tabar A I, Lizaso M T, García B E, Gómez B, Echechipía S, Aldunate M T, Madariaga B, Martínez A. Double-blind, placebo-controlled study of Alternaria alternata immunotherapy: clinical efficacy and safety. Pediatr Allergy Immunol. 2008; 19: 67-75.

12. Zapatero L, Martinez-Cañavate A, Lucas J M, Guallar I, Torres J, Guardia P, de la Torre F, Pedemonte C. Clinical evolution of patients with respiratory allergic disease due to sensitization to Alternaria alternata being treated with subcutaneous immunotherapy. Allergol Immunopathol (Madr) 2011; 39: 79-84.

13. Schütze N, Lehmann I, Bönisch U, Simon J C, Polte T. Exposure to mycotoxins increases the allergic immune response in a murine asthma model. Am J Respir Crit Care Med. 2010; 181:1188-99.

14. Moldaver D, Larché M. Immunotherapy with peptides. Allergy 2011; 66: 784-91.

15. Casale T B, Stokes J R. Future forms of immunotherapy. J Allergy Clin Immunol 2011; 127: 8-15.

16. Grönlund H, Saarne T, Gafvelin G, van Hage M. The major cat allergen, Fel d 1, in diagnosis and therapy. Int Arch Allergy Immunol 2010; 151: 265-74.

17. Müller U, Akdis C A, Fricker M, Akdis M, Blesken T, Bettens F, Blaser K. Successful immunotherapy with T cell epitope peptides of bee venom phospholipase A2 induces specific T cell anergy in patients allergic to bee venom. J Allergy Clin Immunol 1998; 101: 747-54.

18. Postigo I, Gutiérrez-Rodríguez A, Fernández J, Guisantes J A, Suñén E, Martínez J. Diagnostic value of Alt a 1, fungal enolase and manganese-dependent superoxide dismutase in the component-resolved diagnosis of allergy to Pleosporaceae. Clin Exp Allergy 2011; 41: 443-51.

19. Oseroff C, Sidney J, Vita R, Tripple V, McKinney D M, Southwood S, Brodie T M, Sallusto F, Grey H, Alam R, Broide D, Greenbaum J A, Kolla R, Peters B, Sette A. T cell responses to known allergen proteins are differently polarized and account for a variable fraction of total response to allergen extracts. J Immunol 2012; 189: 1800-11.

20. Dordal M T, Lluch-Bernal M, Sánchez M C, Rondón C, Navarro A, Montoro J, Matheu V, Ibá\ddot{n}ez M D, Fernández-Parra B, Dávila I, Conde J, Antón E, Colás C, Valero A. SEAIC Rhinoconjunctivitis Committee. J Investig Allergol Clin Immunol 2011; 21:1-12.

21. Singh H, Raghava G P S. ProPred: Prediction of HLA-DR binding sites. Bioinformatics 2001; 17: 1236-7.

22. Nielsen M, Lundegaard C, Justesen S, Lund O, Buus S. NetMHCIIpan-2.0—Improved pan-specific HLA-DR predictions using a novel concurrent alignment and weight optimization training procedure. Immunome Res 2010; 6: 9.

23. Klitz W, Maiers M, Spellman S, Baxter-Lowe L A, Schmeckpeper B, Williams T M, Fernandez-Viña M. New HLA haplotype frequency reference standards: high-resolution and large sample typing of HLA DR-DQ haplotypes in a sample of European Americans. Tissue Antigens 2003; 62: 296-307.

24. Nielsen M, Lund O. An artificial neural network-based alignment algorithm for MHC class II peptide binding prediction. BMC Bioinformatics 2009; 10: 296.

25. Nielsen M, Lundegaard C, Lund O. Prediction of MHC class II binding affinity using SMM-align, a novel stabilization matrix alignment method. BMC Bioinformatics 2007; 8: 238.

26. Wilcoxon F. Individual comparisons by ranking methods. Biometrics Bulletin 1945; 1: 80-3.

27. Iglewicz B, Banerjee S. “A simple univariate outlier identification procedure.” In Proceedings of the Annual Meeting of the American Statistical Association 2001.

28. Hopp T P, Woods K R. Prediction of protein antigenic determinants from amino acid sequences. Proc Natl Acad Sci USA 1981; 78: 3824-8.

29. Kang H K, Mikszta J A, Deng H, Sercarz E E, Jensen P E, Kim B S. Processing and reactivity of T cell epitopes containing two cysteine residues from hen egg-white lysozyme (HEL74-90). J Immunol 2000; 164: 1775-82.

30. Hague M A, Hawes J W, Blum J S. Cysteinylation of MHC class II ligands: peptide endocytosis and reduction within APC influences T cell recognition. J Immunol 2001; 166: 4543-51.

31. Abulafia-Lapid R, Elias D, Raz I, Keren-Zur Y, Atlan H, Cohen I R. T cell proliferative responses of type 1 diabetes patients and healthy individuals to human hsp60 and its peptides. JAI 1999; 12: 121-9.

32. Kim Y, Berry A H, Spencer D S, Stites W E. Comparing the effect on protein stability of methionine oxidation versus mutagenesis: steps toward engineering oxidative resistance in proteins. Protein Eng 2001; 14: 343-7.

33. Niederberger V, Horak F, Vrtala S, Spitzauer S, Krauth M T, Valent P, Reisinger J, Pelzmann M, Hayek B, Kronqvist M, Gafvelin G, Grönlund H, Purohit A, Suck R, Fiebig H, Cromwell O, Pauli G, van Hage-Hamsten M, Valenta R. Vaccination with genetically engineered allergens prevents progression of allergic disease. Proc Natl Acad Sci USA 2004; 101 (Suppl 2): 14677-82.

34. Maguire P, Nicodemus C, Robinson D, Aaronson D, Umetsu D T. The safety and efficacy of ALLERVAX CAT in cat allergic patients. Clin Immunol 1999; 93: 222-31.

35. Worm M, Lee H H, Kleine-Tebbe J, Hafner R P, Laidler P, Healey D, Buhot C, Verhoef A, Maillère B, Kay A B, Larché M. Development and preliminary clinical evaluation of a peptide immunotherapy vaccine for cat allergy. J Allergy Clin Immunol 2011; 127: 89-97.

36. Immonen A, Farci S, Taivainen A, Partanen J, Pouvelle-Moratille S, Närvänen A, Kinnunen T, Saarelainen S, Rytkönen-Nissinen M, Maillere B, Virtanen T. T cell epitope-containing peptides of the major dog allergen Can f 1 as candidates for allergen immunotherapy. J Immunol 2005; 175: 3614-20.

37. Prickett S R, Voskamp A L, Dacumos-Hill A, Symons K, Rolland J M, O'Hehir R E. Ara h 2 peptides containing dominant CD4+ T-cell epitopes: candidates for a peanut allergy therapeutic. J Allergy Clin Immunol 2011; 127: 608-15.

38. Dimitrov I, Garnev P, Flower D R, Doytchinova I. MHC Class II Binding Prediction-A Little Help from a Friend. J Biomed Biotechnol 2010; 2010: Article ID 705821.

39. Lundegaard C, Lund O, Nielsen M. Predictions versus high-throughput experiments in T-cell epitope discovery: competition or synergy? Expert Rev Vaccines 2012; 11: 43-54.

40. Nielsen M, Lund O, Buus S, Lundegaard C. MHC class II epitope predictive algorithms. Immunol 2010; 130: 319-28.

41. de Lalla C, Sturniolo T, Abbruzzese L, Hammer J, Sidoli A, Sinigaglia F, Panina-Bordignon P. Cutting edge: identification of novel T cell epitopes in Lol p5a by computational prediction. J Immunol 1999; 163: 1725-9.

42. Oseroff C, Sidney J, Tripple V, Grey H, Wood R, Broide D H, Greenbaum J, Kolla R, Peters B, Pomés A, Sette A. Analysis of T cell responses to the major allergens from German cockroach: epitope specificity and relationship to IgE production. J Immunol 2012; 189: 679-88.

43. Pascal M, Konstantinou G, Masilamani M, Lieberman J, Sampson H. In silico prediction of Ara h 2 T cell epitopes in peanut-allergic children. Clin Exper Allergy 2013; 43: 116-127.

44. Sturniolo T, Bono E, Ding J, Raddrizzani L, Tuereci O, Sahin U, Braxenthaler M, Gallazzi F, Protti M P, Sinigaglia F, Hammer J. Generation of tissue-specific and promiscuous HLA ligand databases using DNA microarrays and virtual HLA class II matrices. Nat Biotechnol 1999; 17: 555-61.

45. Zhang L, Chen Y, Wong H S, Zhou S, Mamitsuka H, Zhu S. TEPITOPEpan: extending TEPITOPE for peptide binding prediction covering over 700 HLA-DR molecules. PLoS One 2012; 7: e30483.

46. Slota M, Lim J B, Dang Y, Disis M L. ELISpot for measuring human immune responses to vaccines.

Expert Rev Vaccines 2011; 10: 299-306.

47. Gill D K, Huang Y, Levine G L, Sambor A, Carter D K, Sato A, Kopycinski J, Hayes P, Hahn B, Birungi J, Tarragona-Fiol T, Wan H, Randles M, Cooper A R, Ssemaganda A, Clark L, Kaleebu P, Self S G, Koup R, Wood B, McElrath M J, Cox J H, Hural J, Gilmour J. Equivalence of ELISpot assays demonstrated between major HIV network laboratories. PLoS One 2010; 5: e14330.

48. Moodie Z, Price L, Gouttefangeas C, Mander A, Janetzki S, Löwer M, Welters M J, Ottensmeier C, van der Burg S H, Britten C M. Response definition criteria for ELISPOT assays revisited. Cancer Immunol Immunother 2010; 59: 1489-501.

49. Dittrich M, Lehmann P V. Statistical analysis of ELISPOT assays. Methods Mol Biol 2012; 792: 173-83.

50. Dong X, An B, Salvucci Kierstead L, Storkus W J, Amoscato A A, Salter R D. Modification of the amino terminus of a class II epitope confers resistance to degradation by CD13 on dendritic cells and enhances presentation to T cells. J Immunol 2000; 164: 129-35.

51. Hebart H, Bollinger C, Fisch P, Sarfati J, Meisner C, Baur M, Loeffler J, Monod M, Latgé J P, Einsele H. Analysis of T-cell responses to Aspergillus fumigatus antigens in healthy individuals and patients with hematologic malignancies. Blood 2002; 100: 4521-8.

52. Chaudhary N, Staab J F, Marr K A. Healthy human T-Cell Responses to Aspergillus fumigatus antigens. PLoS One 2010; 5: e9036.

53. Gallelli B, Viviani M, Nebuloni M, Marzano A V, Pozzi C, Messa P, Fogazzi G B. Skin infection due to Alternaria species in kidney allograft recipients: report of a new case and review of the literature. J Nephrol 2006; 19: 668-72.

54. Kovjazin R, Volovitz I, Daon Y, Vider-Shalit T, Azran R, Tsaban L, Carmon L, Louzoun Y. Signal peptides and trans-membrane regions are broadly immunogenic and have high CD8+ T cell epitope densities: Implications for vaccine development. Mol Immunol 2011; 48: 1009-18.

55. Varshaysky A. The N-end rule pathway of protein degradation. Gene Cells 1997; 2: 13-28.

56. De Luca A, Iannitti R G, Bozza S, Beau R, Casagrande A, D'Angelo C, Moretti S, Cunha C, Giovannini G, Massi-Benedetti C, Carvalho A, Boon L, Latgé J P, Romani L. CD4(+) T cell vaccination overcomes defective cross-presentation of fungal antigens in a mouse model of chronic granulomatous disease. J Clin Invest 2012; 122: 1816-31.

57. Schubert M S, Hutcheson P S, Graff R J, Santiago L, Slavin R G. HLA-DQB1 *03 in allergic fungal sinusitis and other chronic hypertrophic rhinosinusitis disorders. J Allergy Clin Immunol 2004; 114: 1376-83.

58. Montone K T, Livolsi V A, Feldman M D, Palmer J, Chiu A G, Lanza D C, Kennedy D W, Loevner L A, Nachamkin I. Fungal rhinosinusitis: a retrospective microbiologic and pathologic review of 400 patients at a single university medical center. Int J Otolaryngol 2012; 2012: 684835.

59. Jorgensen L, Hostrup S, Moeller E H, Grohganz H. Recent trends in stabilising peptides and proteins in pharmaceutical formulation—considerations in the choice of excipients. Expert Opin Drug Deliv 2009; 6: 1219-30.

60. Griffin B T, O'Driscoll C M. Opportunities and challenges for oral delivery of hydrophobic versus hydrophilic peptide and protein-like drugs using lipid-based technologies. Ther Deliv 2011; 2: 1633-53.

61. El-Manzalawy Y, Dobbs D, Honavar V. Predicting flexible length linear B-cell epitopes. Comput Syst Bioinformatics Conf 2008; 7: 121-32.

62. Kasson P M, Rabinowitz J D, Schmitt L, Davis M M, McConnell H M. Kinetics of peptide binding to the class II MHC protein I-Ek. Biochemistry 2000; 39:1048-58.

63. Lippolis J D, White F M, Marto J A, Luckey C J, Bullock T N, Shabanowitz J, Hunt D F, Engelhard V H. Analysis of MHC class II antigen processing by quantitation of peptides that constitute nested sets. J Immunol 2002; 169: 5089-97.

64. Gao J, Faraggi E, Zhou Y, Ruan J, Kurgan L. BEST: improved prediction of B-cell epitopes from antigen sequences. PLoS One 2012; 7: e40104.

65. O'Brien C, Flower D R, Feighery C. Peptide length significantly influences in vitro affinity for MHC class II molecules. Immunome Res 2008; 4: 6.

66. Carson R T, Vignali K M, Woodland D L, Vignali D A. T cell receptor recognition of MHC class II-bound peptide flanking residues enhances immunogenicity and results in altered TCR V region usage. Immunity 1997; 7: 387-99.

67. Arnold P Y, La Gruta N L, Miller T, Vignali K M, Adams P S, Woodland D L, Vignali D A. The majority of immunogenic epitopes generate CD4+ T cells that are dependent on MHC class II-bound peptide-flanking residues. J Immunol 2002; 169: 739-49.

1. A composition comprising at least two peptides, each of said at least two peptides selected from a different one of groups (i) to (vii) wherein a peptide consists of or comprises the amino acid sequence defined by the respective SEQ ID NO, and wherein each peptide has an amino acid length of from 8 to 50 amino acids
(i) SEQ ID NO: 2, SEQ ID NOs: 27-41
(\ddot{v}) SEQ ID NO: 4, SEQ ID NOs: 42-56
(Hi) SEQ ID NO: 5, SEQ ID NOs: 57-71
(iv) SEQ ID NO: 1, SEQ ID NOs: 72-86
(v) SEQ ID NO: 12, SEQ ID NOs: 87-101
(vi) SEQ ID NO: 20, SEQ ID NOs: 102-16
(vii) SEQ ID NO: 21 , SEQ ID NOs: 117-139.

2. The composition of claim 1 which is selected from:
(a) the composition of claim 1 wherein each peptide has a maximum length of 15 amino acids and a minimum length of 9 amino acids,
(b) the composition of claim 1 having at least one peptide from group (iii),
(c) the composition of claim 1 having at least one peptide from each of groups (iii) and (i),
(d) the composition of claim 1 having at least one peptide from each of groups (iii), (ii) and (iv),
(e) the composition of claim 1 having at least one peptide from each of groups (iii), (ii) and (v),
(f) the composition of claim 1 having at least one peptide from each of groups (iii), (ii) and (vi),
(g) the composition of claim 1 having at least one peptide from each of groups (iii), (iv) and (vii),
(h) the composition of claim 1 having at least three, four, five, six or seven peptides, wherein each peptide is from a different one of groups (i) to (vii), and
(i) the composition of claim 1 having seven peptides, wherein each peptide is from a different one of groups (i) to (vii).

3-10. (canceled)

11. A method for treatment of disease comprising
simultaneous, sequential or separate administration of at least two peptides selected from one of groups (i) to (vii), each of said at least two peptides selected from a different one of groups (i) to (vii), wherein each peptide consists of or comprises the amino acid sequence defined by the respective SEQ ID NO, and wherein each peptide has an amino acid length of from 8 to 50 amino acids, wherein groups (i) to (vii) are:
(i) SEQ ID NO: 2, SEQ ID NOs: 27-41,
(ii) SEQ ID NO: 4, SEQ ID NOs: 42-56,
(iii) SEQ ID NO: 5, SEQ ID NOs: 57-71,
(iv) SEQ ID NO: 11 , SEQ ID NOs: 72-86,
(v) SEQ ID NO: 12, SEQ ID NOs: 87-101,
(vi) SEQ ID NO: 20, SEQ ID NOs: 102-116, and
(vii) SEQ ID NO: 21 , SEQ ID NOs: 117-139.

12. (canceled)

13. The method of claim 11 in which at least one of:
(a) each peptide has a maximum length of 15 amino acids and a minimum length of 9 amino acids,
(b) at least one peptide is from group (iii),
(c) at least one peptide is from each of groups (iii) and (i),
(d) at least one peptide is from each of groups (iii), (ii) and (iv),
(e) at least one peptide is from each of groups (iii), (ii) and (v),
(f) at least one peptide is from each of groups (iii), (ii) and (vi),
(g) at least one peptide is from each of groups (iii), (iv) and (vii),
(h) at least three, four, five, six or seven peptides are administered, and wherein each said peptide is from a different one of groups (i) to (vii),
(i) seven peptides are administered, and wherein each peptide is from a different one of groups (i) to (vii),
(j) at least two of the peptides are administered in a combined preparation, or
(k) the disease is an allergic disease, optionally chosen from fungal allergy, fungal asthma, fungal infection, SAFS, ABPA, Aspergillosis or an allergic disease caused by or in which the patient is sensitised to Alternaria alternata and/or to one or both of Alt a 1 or Alt a 5.

14-23. (canceled)

24. A method for the production of a pharmaceutical composition or medicament, the method comprising
mixing the composition of claim 1 with a pharmaceutically acceptable carrier, adjuvant or diluent.

25. A peptide consisting of or comprising the amino acid sequence of one of:
(a) SEQ ID NO: 2, SEQ ID NOs: 31, 33, 35, 36, 38, 39,
(b) SEQ ID NO: 8, SEQ ID NOs: 140-54,
(c) SEQ ID NO: 9, SEQ ID NOs: 155-169,
(d) SEQ ID NO: 26, SEQ ID NOs: 170-184,
or a peptide having a contiguous amino acid sequence having at least 70%, 80%, 85%, 90% or 95% sequence identity to the amino acid sequence of one of said SEQ ID NOs, wherein the peptide has an amino acid length of from 8 to 50 amino acids, wherein the peptide is not one of SEQ ID NOs: 27, 28, 29, 30, 32, 34, 37, 40 or 41.

26. (canceled)

27. The peptide of claim 25 having a maximum length of 15 amino acids and a minimum length of 9 amino acids.

28. A pharmaceutical composition comprising the peptide of claims 25; and a pharmaceutically acceptable carrier, adjuvant or diluent.

29-32. (canceled)

33. A method of treating or preventing disease in a patient in need of treatment thereof, the method comprising administering to the patient a therapeutically effective amount of the pharmaceutical composition of claim 28.

34. (canceled)

35. A nucleic acid encoding the peptide of claim 25.

36. A cell having integrated in its genome the nucleic acid of claim 35 operably linked to a transcription control nucleic acid sequence.

37. A nucleic acid expression vector comprising the nucleic acid of claim 35 operably linked to a transcription control nucleic acid sequence, wherein the vector is configured for expression of the encoded peptide when transfected into a suitable cell.

38. A cell transfected with the nucleic acid expression vector of claim 37.

39. A method of identifying a peptide that is capable of stimulating an immune response, the method comprising the steps of:
providing a candidate peptide having a contiguous amino acid sequence having at least 70% sequence identity to the amino acid sequence of one of:
(a) SEQ ID NO: 2, SEQ ID NOs: 31, 33, 35, 36, 38, 39,
(b) SEQ ID NO: 8, SEQ ID NOs: 140-154,
(c) SEQ ID NO: 9, SEQ ID NOs: 155-169, or
(d) SEQ ID NO: 26, SEQ ID NOs: 170-184,
and wherein the peptide is optionally not one of SEQ ID NOs: 27, 28, 29, 30, 32, 34, 37, 40 or 41; and
(ii) testing an ability of the candidate peptide to induce an immune response.

40. The method of claim 39 wherein step (i) comprises providing a peptide having the amino acid sequence of one of said SEQ ID NOs and chemically modifying the peptide to provide the candidate peptide.

41. The method of claim 39 wherein either one or both of:
step (i) comprises providing a peptide having the amino acid sequence of one of said SEQ ID NOs and chemically modifying the peptide to provide the candidate peptide, and
step (ii) comprises contacting the candidate peptide with a population of T cells in vitro and assaying T cell proliferation.

42. The method of claim 39 wherein either one or both of:
step (i) comprises providing a peptide having the amino acid sequence of one of said SEQ ID NOs and chemically modifying the peptide to provide the candidate peptide, and
step (ii) comprises monitoring for production of IL-4 and/or IFNγ.
